(19) United States
(12) Patent Application Publication
(10) Pub. No.: US 2023/0399303 A1
(43) Pub. Date: Dec. 14, 2023

(54) Macrophage Migration Inhibitory Factor Inhibitors, and Methods of Making and Using Same

(71) Applicant: YALE UNIVERSITY, New Haven, CT (US)

(72) Inventor: William L. Jorgensen, Deep River, CT (US)

(21) Appl. No.: 18/250,530

(22) PCT Filed: Oct. 26, 2021
(86) PCT No.: PCT/US2021/056646

§ 371 Date: Apr. 25, 2023

Related U.S. Application Data
(60) Provisional application No. 63/105,477, filed on Oct. 26, 2020.

(51) Int. Cl. C07D 265/18; C07D 413/12

(52) U.S. Cl. CPC C07D 265/18; C07D 413/12

(57) ABSTRACT

The present disclosure provides inhibitors of MIF tautomerase activity. In certain embodiments, the compounds inhibitors are useful in treating or preventing inflammatory and/or auto-immune diseases. In other embodiments, the compounds are useful in reversing, ameliorating, and/or preventing tumor growth. In yet other embodiments, the compounds are useful in reversing, ameliorating, and/or preventing angiogenesis.

### CROSS-REFERENCE TO RELATED APPLICATION

This application claims priority to U.S. Provisional Patent Application Ser. No. 63/105,477 filed Oct. 26, 2021, entitled “MACROPHAGE MIGRATION INHIBITORY FACTOR INHIBITORS, AND METHODS OF MAKING AND USING SAME,” the disclosure of which is incorporated herein by reference in its entirety.

### STATEMENT REGARDING FEDERALLY SPONSORED RESEARCH

This invention was made with government support under GM032136 awarded by National Institutes of Health. The government has certain rights in the invention.

### BACKGROUND

Macrophage migration inhibitory factor (MIF) is a cytokine that plays a central role in numerous inflammatory diseases. MIF is widely expressed in both immune and non-immune cells including macrophages, endothelial cells, and T-cells. Upon activation, the cells release MIF, which promotes the release of other inflammatory cytokines such as TNF-α and IL-1.

Excessive or chronic inflammatory response is associated with tissue damage and autoimmune diseases such as rheumatoid arthritis, Crohn's disease, and lupus erythematosus.

The connection between inflammatory disease and cancer is also well-established, and MIF has been shown to enhance cell proliferation by inhibiting accumulation of the tumor suppressor p53 and by promotion of angiogenesis. High MIF levels are also associated with numerous neurological disorders, including Alzheimer's disease.

MIF is over-expressed in many cancer cells and can serve as a marker for disease progression. Furthermore, MIF in cancer cells is protected from degradation by Hsp90, which has led to proposed targeting of Hsp90 as an indirect way of inhibiting MIF function. Disruption of the inflammatory cascade and restoration of normal p53 levels have clear implications for the potential therapeutic value of inhibitors of MIF signaling. Indeed, immunoneutralization of MIF or deletion of the MIF gene is known to suppress inflammatory response, tumor growth, and angiogenesis. At the molecular level, what is needed is interference with the interaction between MIF and its cell-surface receptor CD74.

MIF is a toroid-shaped, trimeric protein with a total of 342 amino acid residues. Besides its role as a cytokine, MIF is a keto-enol tautomerase. Though the enzymatic activity appears to be vestigial in humans, there are three tautomerase active sites at the interfaces of the monomer units opening to the outside of the toroid. The presence of the tautomerase sites presents an opportunity for complexation of a tautomerase inhibitor that can also interfere with MIF/CD74 binding.

There is thus a need in the art for identifying novel compounds that inhibit MIF tautomerase activity. In certain embodiments, such compounds can be used to treat inflammatory and/or auto-immune diseases, and reverse, ameliorate and/or prevent tumor growth and angiogenesis. The present disclosure addresses this need.

### BRIEF SUMMARY OF THE INVENTION

In one aspect, a compound of formula (I), or a salt, enantiomer, tautomer, or solvate thereof, is provided and has the structure:

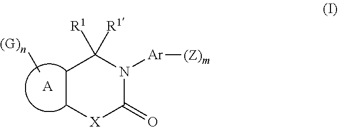

wherein:
R¹ and R^{1′} are each independently H, C₁₋₃ alkyl, or halogen;
X is CH₂, O, or S;
A is a fused 5-membered heteroaryl ring or a fused 6-membered aryl or heteroaryl ring;
each G is independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C10 alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, and combinations thereof;
Ar is a 5-membered heteroaryl or a 6-membered aryl or heteroaryl ring;
each Z is a substituent on Ar independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, —(OCH₂CH₂)ₚ-4-10 membered heterocyclyl, —O(CH₂CH₂O)ₚ-4-10 membered heterocyclyl, —(OCH₂CH₂)ₚ-4-10 membered heteroaryl, —O(CH₂CH₂O)ₚ-4-10 membered heteroaryl, —O(CH₂CH₂O)ₚ—C₁₋₄ alkyl, and combinations thereof;
each R and R′ is independently H or C₁₋₁₀ hydrocarbyl;
n is an integer from 1 to 4;
m is an integer from 1 to 5; and
p is an integer from 1 to 5;
with the proviso that if
   Ar is phenyl; A is a fused 6-membered aryl ring having the structure

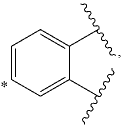 [SMILES: *c1ccccc1*]

substituted at the position marked with * by hydrogen, hydroxy, C₁₋₅ alkoxy, C₁₋₅ haloalkyl, or C₁₋₅ haloalkoxy; and m is 1; then Z is not OH, C₁₋₅ alkyl, C₁₋₅ alkoxy, C₁₋₅ haloalkyl, C₁₋₅ haloalkoxy, OCF₃, phenyl, or halogen.

Compounds of formula (I) are MIF inhibitors, and in some aspects, are useful in a method of treating a disease or condition in which inhibition of macrophage migration inhibitory factor (MIF) activity in a subject is therapeutically beneficial, the method comprising administering to the subject an effective amount of a composition comprising the compound of formula (I), or a salt, enantiomer, tautomer, or solvate thereof,

wherein the disease or condition is selected from the group consisting of an inflammatory disease, an autoimmune disease, and cancer.

### BRIEF DESCRIPTION OF THE FIGURES

The drawings illustrate generally, by way of example, but not by way of limitation, various embodiments of the present application.

FIG. 1 shows a rendering of the X-ray crystal structure of compound NVS-2 bound to human MIF (PDB code 5HVT).

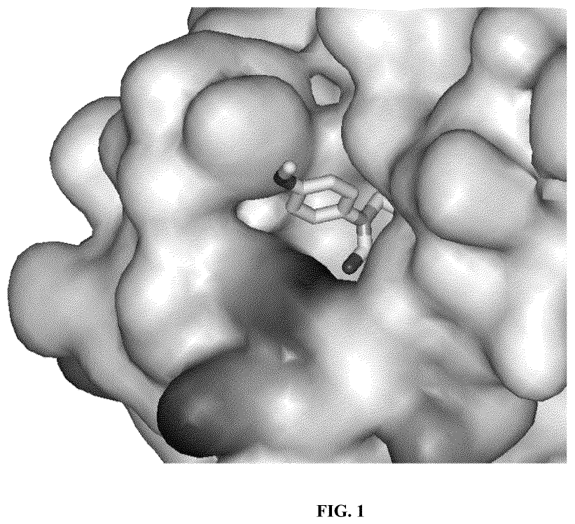

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “haloalkoxy” as used herein refers to an alkoxy group that has one or more hydrogen atoms substituted with a halogen.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroalkyl” group, as used herein, refers to an alkyl or hydrocarbyl group that has one or more hydrogen atoms or carbon atoms replaced by a heteroatom. Alkoxy groups, haloalkyl groups are specific non-limiting examples of heteroalkyl groups. Other examples of heteroalkyl groups include polyethylene glycols, ethers, thioethers, amino alkyl groups, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxy cyclohexyl)ethyl, 2-(2,3-epoxy cylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

Compounds of formula (I) or otherwise described herein can be prepared by the general schemes described herein, using synthetic methods known by those skilled in the art. The following examples illustrate non-limiting embodiments of the compound(s) described herein and their preparation.

In various embodiments, compounds for formula (I) can be synthesized according to Schemes 1 and 2.

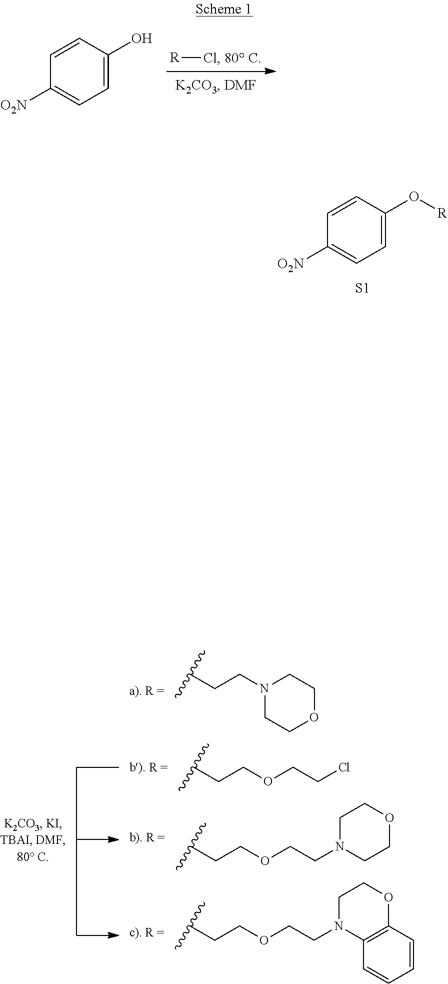

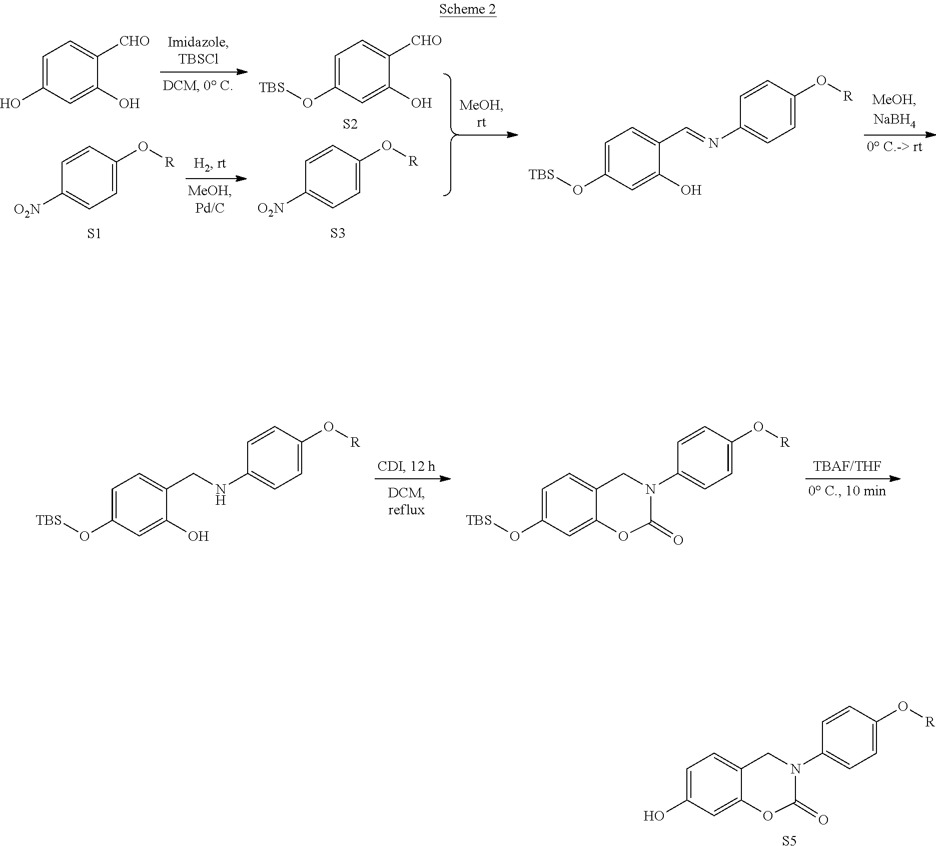

In various embodiments, a compound of formula (I), or a salt, enantiomer, tautomer, or solvate thereof is provided. The compound of formula (I) has the structure:

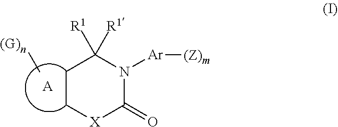

wherein:
R¹ and R^{1′} are each independently H, C₁₋₃ alkyl, or halogen;
X is CH₂, O, or S;
A is a fused 5-membered heteroaryl ring or a fused 6-membered aryl or heteroaryl ring;
each G is independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, and combinations thereof;
Ar is a 5-membered heteroaryl or a 6-membered aryl or heteroaryl ring; each Z is a substituent on Ar independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, —(OCH₂CH₂)ₚ-4-10 membered heterocyclyl, —O(CH₂CH₂O)ₚ-4-10 membered heterocyclyl, —(OCH₂CH₂)ₚ-4-10 membered heteroaryl, —O(CH₂CH₂O)ₚ-4-10 membered heteroaryl, —O(CH₂CH₂O)ₚ—C₁₋₄ alkyl, and combinations thereof,
each R and R′ is independently H or C₁₋₁₀ hydrocarbyl;
n is an integer from 1 to 4;
m is an integer from 1 to 5; and
p is an integer from 1 to 5;
   with the proviso that if Ar is phenyl; A is a fused 6-membered aryl ring having the structure

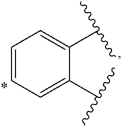 [SMILES: *c1ccccc1*]

substituted at the position marked with * by hydrogen, hydroxy, C₁₋₅ alkoxy, C₁₋₅ haloalkyl, or C₁₋₅ haloalkoxy; and m is 1; then Z is not OH, C₁₋₅ alkyl, C₁₋₅ alkoxy, C₁₋₅ haloalkyl, C₁₋₅ haloalkoxy, OCF₃, phenyl, or halogen.

In various embodiments, the compound of formula (I) is not
3-(4-methoxyphenyl)-7-hydroxy-3,4-dihydro-benzo[e][1,3]oxazin-2-one,
3-phenyl-7-hydroxy-3,4-dihydro-benzo[e][1,3]oxazin-2-one,
3-(3-hydroxyphenyl)-7-hydroxy-3,4-dihydro-benzo[e][1,3]oxazin-2-one,
3-(4-hydroxyphenyl)-7-hydroxy-3,4-dihydro-benzo[e][1,3]oxazin-2-one,
3-(4-aminosulfanyloxyphenyl)-7-hydroxy-3,4-dihydro-benzo[e][1,3]oxazin-2-one,
3-(phenylmethyl)-7-hydroxy-3,4-dihydro-benzo[e][1,3]oxazin-2-one,
3-(4-trifluoromethyloxyphenyl)-7-hydroxy-3,4-dihydro-benzo[e][1,3]oxazin-2-one,
3-(4-methoxyphenyl)-3,4-dihydro-benzo[e][1,3]oxazin-2-one,
3-(biphenyl-4-yl)-3,4-dihydro-benzo[e][1,3]oxazin-2-one,
3-(naphth-1-yl)-7-hydroxy-3,4-dihydro-benzo[e][1,3]oxazin-2-one,
3-(4-bromophenyl))-3,4-dihydro-beπzo[θ][1,3]oxazin-2-one,
3-(3-aminosulfanyloxyphenyl)-7-aminosulfanyloxy-3,4-dihydro-benzo[8][1,3]oxazin-2-one,
3-(4-aminosulfanyloxyphenyl)-7-aminosulfanyloxy-3,4-dihydro-benzo[e][1,3]oxazin-2-one,
3-(4-methoxyphenyl)-7-aminosulfanyloxy-3,4-dihydro-benzo[e][1,3]oxazin-2-one,
3-Phenyl-7-aminosulfanyloxy-3,4-dihydro-benzo[e][1,3]oxazin-2-one,
3-(4-hydroxyphenyl)-7-aminosulfanyloxy-3,4-dihydro-benzo[e][1,3]oxazin-2-one, or
3-(phenylmethyl)-7-aminosulfanyloxy-3,4-dihydro-benzo[e][1,3]oxazin-2-one.

In various embodiments, the compound of formula (I) has the structure

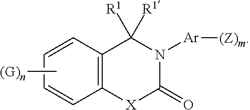

In various embodiments, X is O. In various embodiments, X is CH₂. In various embodiments, X is S. In various embodiments, R¹ and R^{1′} are H.

In various embodiments, the compound of formula (I) has the structure

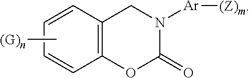

In various embodiments, the compound of formula (I) has the structure

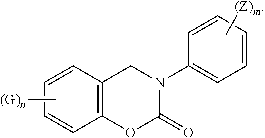

In various embodiments, the compound of formula (I) has the structure

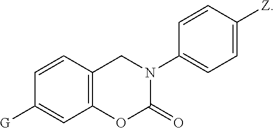

In various embodiments, the compound of formula (I) has the structure

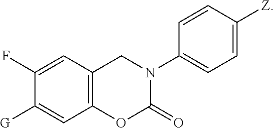

In various embodiments, G is OH.

In various embodiments, Z is selected from the group consisting of —(OCH₂CH₂)ₚ-4-10 membered non-aromatic heterocyclyl, —O(CH₂CH₂O)ₚ-4-10 membered non-aromatic heterocyclyl, —(OCH₂CH₂)ₚ-4-10 membered heteroaryl, —O(CH₂CH₂O)ₚ-4-10 membered heteroaryl, and —O(CH₂CH₂O)ₚ—C₁₋₄ alkyl.

In various embodiments, Z is selected from the group consisting of

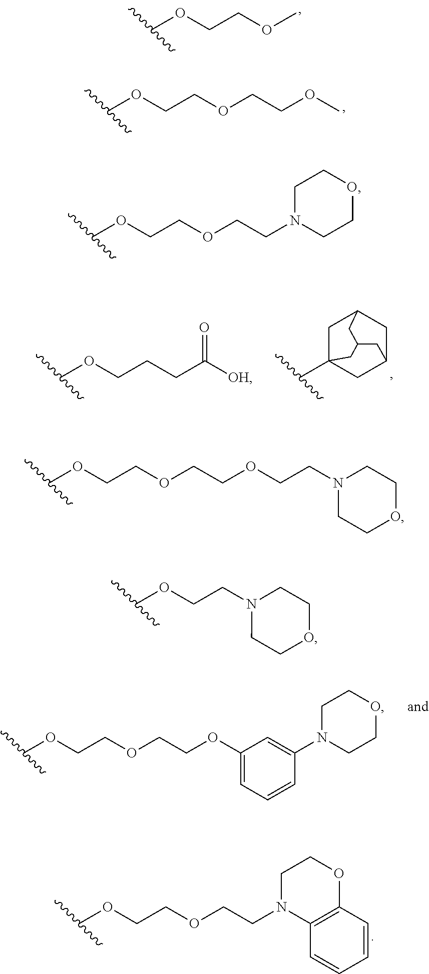

In various embodiments, the compound of formula (I) is selected from the group consisting of:

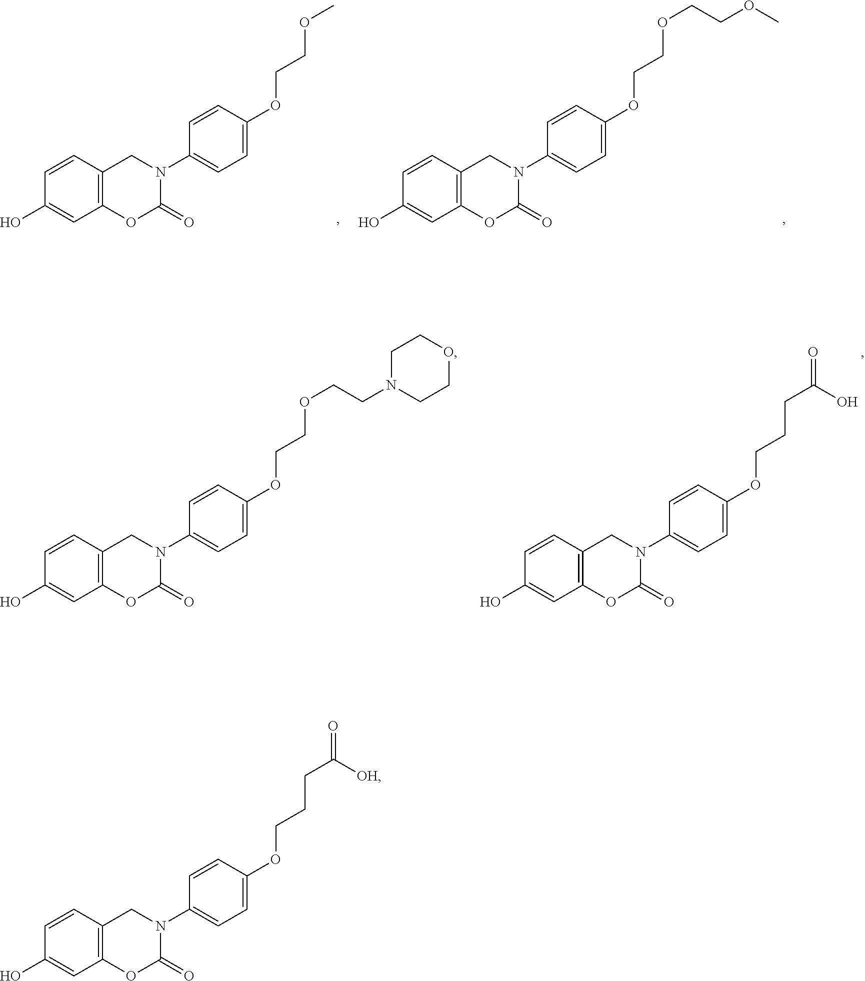 [SMILES: COCCOc1ccc(N2Cc3ccc(O)cc3OC2=O)cc1 ; COCCOCCOc1ccc(N2Cc3ccc(O)cc3OC2=O)cc1 ; O=C1Oc2cc(O)ccc2CN1c1ccc(OCCOCCN2CCOCC2)cc1 ; O=C(O)CCCOc1ccc(N2Cc3ccc(O)cc3OC2=O)cc1 ; O=C(O)CCCOc1ccc(N2Cc3ccc(O)cc3OC2=O)cc1]

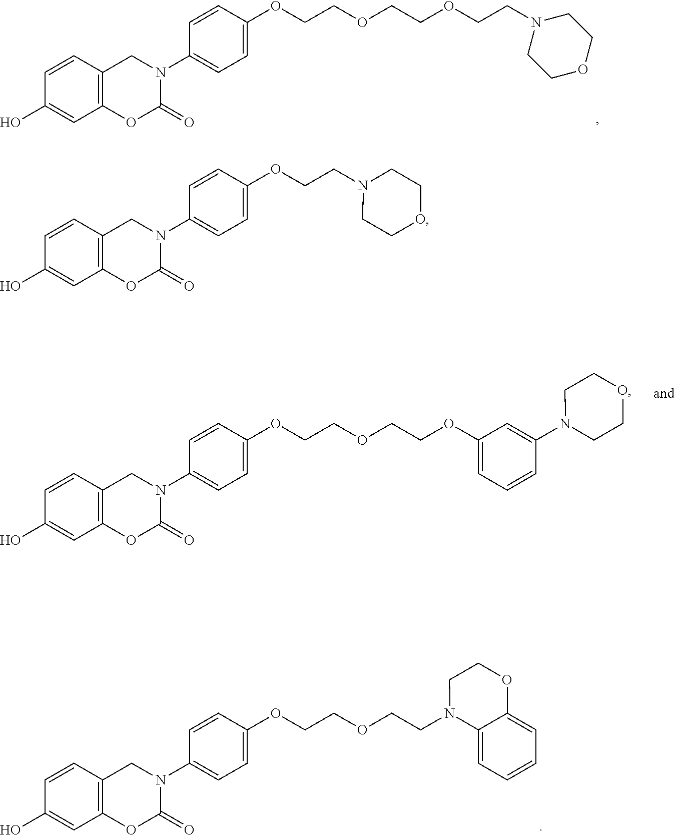 [SMILES: O=C1Oc2cc(O)ccc2CN1c1ccc(OCCOCCOCCN2CCOCC2)cc1 ; O=C1Oc2cc(O)ccc2CN1c1ccc(OCCN2CCOCC2)cc1 ; O=C1Oc2cc(O)ccc2CN1c1ccc(OCCOCCOc2cccc(N3CCOCC3)c2)cc1 ; O=C1Oc2cc(O)ccc2CN1c1ccc(OCCOCCN2CCOc3ccccc32)cc1]

In various embodiments, Z is selected from any one of the groups in Table 1 below:

TABLE 1
Water solubilizing groups (Z) in the compound of formula (I).

| | —CH₂CH₂OCH₂CH₂NMe₂ |
|---|---|
| —OH | 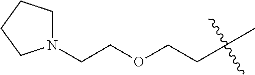 [SMILES: *CCOCCN1CCCC1] |
| —OEt | 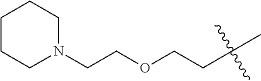 [SMILES: *CCOCCN1CCCCC1] |
| —OPr | 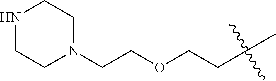 [SMILES: *CCOCCN1CCNCC1] |
| —OiPr | 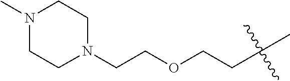 [SMILES: *CCOCCN1CCN(C)CC1] |
| —OcPr | 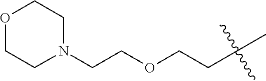 [SMILES: *CCOCCN1CCOCC1] |
| —OcBu | —CH₂CH₂NHCH₂CH₂OH |
| —OcPn | —CH₂CH₂NHCH₂CH₂OMe |
| —OcHex | —CH₂CH₂NHCH₂CH₂OEt |
| —OCH₂CH₂OH | —CH₂CH₂NHCH₂CH₂NH₂ |
| —OCH₂CH₂OMe | —CH₂CH₂NHCH₂CH₂NHMe |
| —OCH₂CH₂OEt | —CH₂CH₂NHCH₂CH₂NMe₂ |
| —OCH₂CH₂NH₂ | 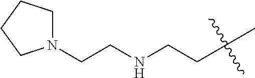 [SMILES: *CCNCCN1CCCC1] |
| —OCH₂CH₂NHMe | 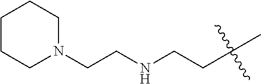 [SMILES: *CCNCCN1CCCCC1] |
| —OCH₂CH₂NMe₂ | 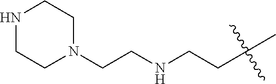 [SMILES: *CCNCCN1CCNCC1] |
| 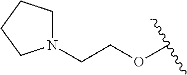 [SMILES: *OCCN1CCCC1] | 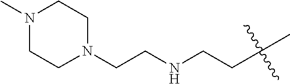 [SMILES: *CCNCCN1CCN(C)CC1] |
| 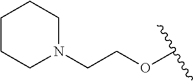 [SMILES: *OCCN1CCCCC1] | 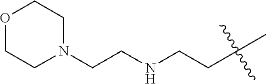 [SMILES: *CCNCCN1CCOCC1] |
| 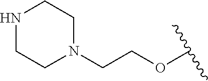 [SMILES: *OCCN1CCNCC1] | —CH₂CH₂N(CH₂CH₂OH)₂ |
| 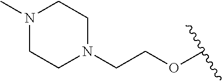 [SMILES: *OCCN1CCN(C)CC1] | —CH₂CH₂N(CH₂CH₂OMe)₂ |
| 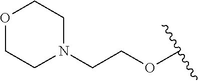 [SMILES: *OCCN1CCOCC1] | —CH₂CH₂N(CH₂CH₂OEt)₂ |
| —OCH₂CH₂CH₂OH | —CH₂CH₂NMeCH₂CH₂OH |
| —OCH₂CH₂CH₂OMe | —CH₂CH₂NMeCH₂CH₂OMe |
| —OCH₂CH₂CH₂OEt | —CH₂CH₂NMeCH₂CH₂OEt |
| —OCH₂CH₂CH₂NH₂ | —CH₂CH₂NMeCH₂CH₂NH₂ |
| —OCH₂CH₂CH₂NHMe | —-CH₂CH₂NMeCH₂CH₂NHMe |
| —OCH₂CH₂CH₂NMe₂ | —CH₂CH₂NMeCH₂CH₂NMe₂ |
| 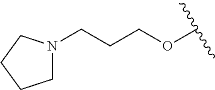 [SMILES: *OCCCN1CCCC1] | 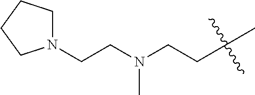 [SMILES: *CCN(C)CCN1CCCC1] |
| 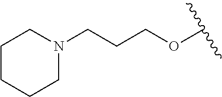 [SMILES: *OCCCN1CCCCC1] | 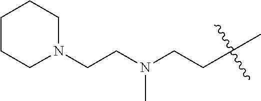 [SMILES: *CCN(C)CCN1CCCCC1] |
| 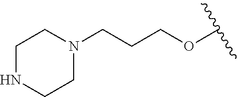 [SMILES: *OCCCN1CCNCC1] | 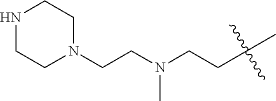 [SMILES: *CCN(C)CCN1CCNCC1] |
| 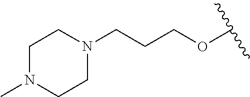 [SMILES: *OCCCN1CCN(C)CC1] | 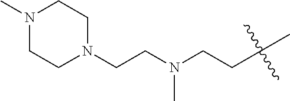 [SMILES: *CCN(C)CCN1CCN(C)CC1] |
| 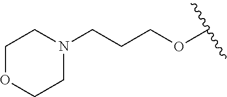 [SMILES: *OCCCN1CCOCC1] | 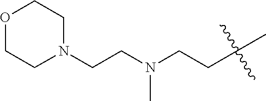 [SMILES: *CCN(C)CCN1CCOCC1] |
| —OCH₂CH₂OCH₂CH₂OH | —NH |
| —OCH₂CH₂OCH₂CH₂OMe | —NHMe |
| —OCH₂CH₂OCH₂CH₂OEt | —NMe₂ |
| —OCH₂CH₂OCH₂CH₂NH₂ | —NHEt |
| —OCH₂CH₂OCH₂CH₂NHMe | —NHPr |
| —OCH₂CH₂OCH₂CH₂NMe₂ | —NHiPr |
| 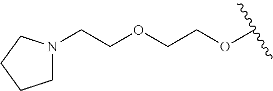 [SMILES: *OCCOCCN1CCCC1] | —NHcPr |
| 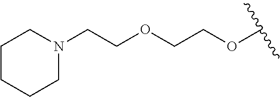 [SMILES: *OCCOCCN1CCCCC1] | —NHcBu |
| 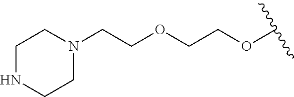 [SMILES: *OCCOCCN1CCNCC1] | —NHcPn |
| 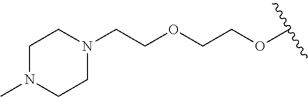 [SMILES: *OCCOCCN1CCN(C)CC1] | —NHcHex |
| 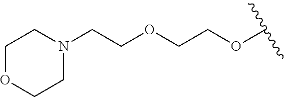 [SMILES: *OCCOCCN1CCOCC1] | —NHCH₂CH₂OH |
| —OCH₂CH₂OCH₂CH₂CH₂OH | —NHCH₂CH₂OMe |
| —OCH₂CH₂OCH₂CH₂CH₂OMe | —NHCH₂CH₂OEt |
| —OCH₂CH₂OCH₂CH₂CH₂OEt | —NHCH₂CH₂NH₂ |
| —OCH₂CH₂OCH₂CH₂CH₂NH₂ | —NHCH₂CH₂NHMe |
| —OCH₂CH₂OCH₂CH₂CH₂NHMe | —NHCH₂CH₂NMe₂ |
| —OCH₂CH₂OCH₂CH₂CH₂NMe₂ | 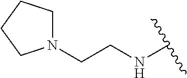 [SMILES: *NCCN1CCCC1] |
| 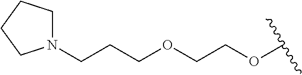 [SMILES: *OCCOCCCN1CCCC1] | 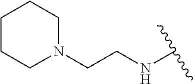 [SMILES: *NCCN1CCCCC1] |
| 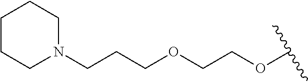 [SMILES: *OCCOCCCN1CCCCC1] | 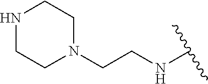 [SMILES: *NCCN1CCNCC1] |
| 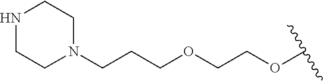 [SMILES: *OCCOCCCN1CCNCC1] | 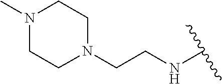 [SMILES: *NCCN1CCN(C)CC1] |
| 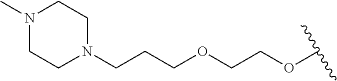 [SMILES: *OCCOCCCN1CCN(C)CC1] | 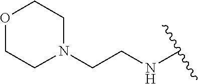 [SMILES: *NCCN1CCOCC1] |
| 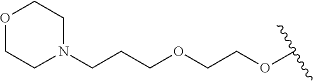 [SMILES: *OCCOCCCN1CCOCC1] | —NHCH₂CH₂CH₂OH |
| —OCH₂CH₂CH₂OCH₂CH₂OH | —NHCH₂CH₂CH₂OMe |
| —OCH₂CH₂CH₂OCH₂CH₂OMe | —NHCH₂CH₂CH₂OEt |
| —OCH₂CH₂CH₂OCH₂CH₂OEt | —NHCH₂CH₂CH₂NH₂ |
| —OCH₂CH₂CH₂OCH₂CH₂NH₂ | —NHCH₂CH₂CH₂NHMe |
| —OCH₂CH₂CH₂OCH₂CH₂NHMe | —NHCH₂CH₂CH₂NMe₂ |
| —OCH₂CH₂CH₂OCH₂CH₂NMe₂ | 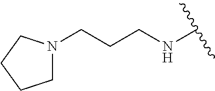 [SMILES: *NCCCN1CCCC1] |
| 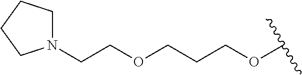 [SMILES: *OCCCOCCN1CCCC1] | 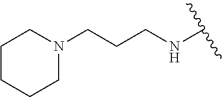 [SMILES: *NCCCN1CCCCC1] |
| 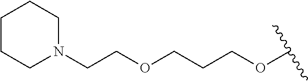 [SMILES: *OCCCOCCN1CCCCC1] | 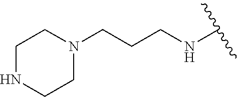 [SMILES: *NCCCN1CCNCC1] |
| 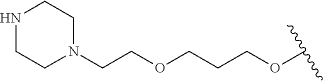 [SMILES: *OCCCOCCN1CCNCC1] | 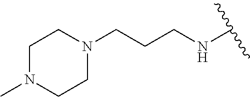 [SMILES: *NCCCN1CCN(C)CC1] |
| 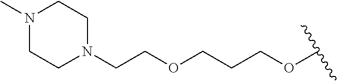 [SMILES: *OCCCOCCN1CCN(C)CC1] | 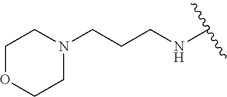 [SMILES: *NCCCN1CCOCC1] |
| 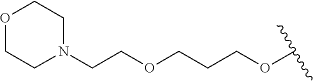 [SMILES: *OCCCOCCN1CCOCC1] | —NHCH₂CH₂OCH₂CH₂OH |
| —OCH₂CH₂CH₂OCH₂CH₂CH₂OH | —NHCH₂CH₂OCH₂CH₂OMe |
| —OCH₂CH₂CH₂OCH₂CH₂CH₂OMe | —NHCH₂CH₂OCH₂CH₂OEt |
| —OCH₂CH₂CH₂OCH₂CH₂CH₂OEt | —NHCH₂CH₂OCH₂CH₂NH₂ |
| —OCH₂CH₂CH₂OCH₂CH₂CH₂NH₂ | —NHCH₂CH₂OCH₂CH₂NHMe |
| —OCH₂CH₂CH₂OCH₂CH₂CH₂NHMe | —NHCH₂CH₂OCH₂CH₂NMe₂ |
| —OCH₂CH₂CH₂OCH₂CH₂CH₂NMe₂ | —N(CH₂CH₂OH)₂ |
| 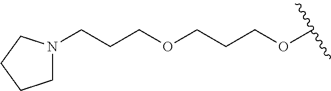 [SMILES: *OCCCOCCCN1CCCC1] | —N(CH₂CH₂OMe)₂ |
| 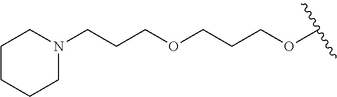 [SMILES: *OCCCOCCCN1CCCCC1] | —N(CH₂CH₂OEt)₂ |
| 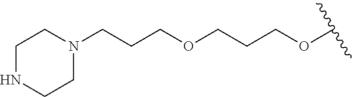 [SMILES: *OCCCOCCCN1CCNCC1] | —N(CH₂CH₂CH₂OH)₂ |
| 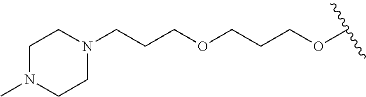 [SMILES: *OCCCOCCCN1CCN(C)CC1] | —N(CH₂CH₂CH₂OMe)₂ |
| 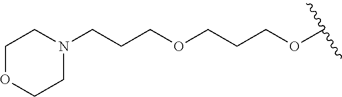 [SMILES: *OCCCOCCCN1CCOCC1] | —N(CH₂CH₂CH₂OEt)₂ |
| —OCH₂CH₂NHCH₂CH₂OH | —N(CH₂CH₂CH₂NH₂)₂ |
| —OCH₂CH₂NHCH₂CH₂OMe | —N(CH₂CH₂CH₂NHMe)₂ |
| —OCH₂CH₂NHCH₂CH₂OEt | —N(CH₂CH₂CH₂NMe₂)₂ |
| —OCH₂CH₂NHCH₂CH₂NH₂ | 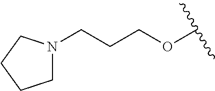 [SMILES: *OCCCN1CCCC1] |
| —OCH₂CH₂NHCH₂CH₂NHMe | 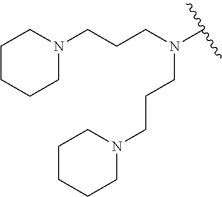 [SMILES: *N(CCCN1CCCCC1)CCCN1CCCCC1] |
| —OCH₂CH₂NHCH₂CH₂NMe₂ | 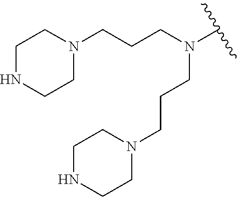 [SMILES: *N(CCCN1CCNCC1)CCCN1CCNCC1] |
| 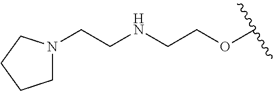 [SMILES: *OCCNCCN1CCCC1] | 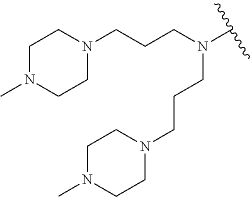 [SMILES: *N(CCCN1CCN(C)CC1)CCCN1CCN(C)CC1] |
| 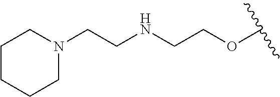 [SMILES: *OCCNCCN1CCCCC1] | 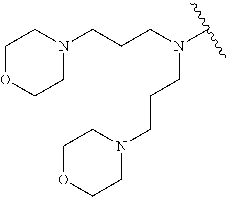 [SMILES: *N(CCCN1CCOCC1)CCCN1CCOCC1] |
| 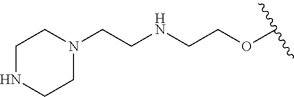 [SMILES: *OCCNCCN1CCNCC1] | —N(CH₂CH₂OCH₂CH₂OH)₂ |
| 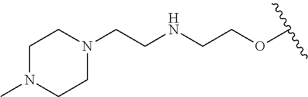 [SMILES: *OCCNCCN1CCN(C)CC1] | —N(CH₂CH₂OCH₂CH₂OMe)₂ |
| 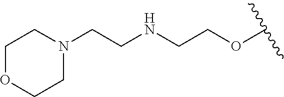 [SMILES: *OCCNCCN1CCOCC1] | —N(CH₂CH₂OCH₂CH₂OEt)₂ |
| —OCH₂CH₂NHCH₂CH₂CH₂OH | —N(CH₂CH₂OCH₂CH₂NH₂)₂ |
| —OCH₂CH₂NHCH₂CH₂CH₂OMe | —N(CH₂CH₂OCH₂CH₂NHMe)₂ |
| —OCH₂CH₂NHCH₂CH₂CH₂OEt | —N(CH₂CH₂OCH₂CH₂NMe₂)₂ |
| —OCH₂CH₂NHCH₂CH₂CH₂NH₂ | —NMeCH₂CH₂OH |
| —OCH₂CH₂NHCH₂CH₂CH₂NHMe | —NMeCH₂CH₂OMe |
| —OCH₂CH₂NHCH₂CH₂CH₂NMe₂ | —NMeCH₂CH₂OEt |
| 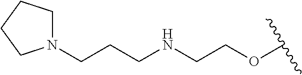 [SMILES: *OCCNCCCN1CCCC1] | —NMeCH₂CH₂NH₂ |
| 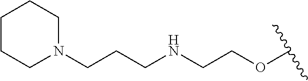 [SMILES: *OCCNCCCN1CCCCC1] | —NMeCH₂CH₂NHMe |
| 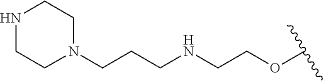 [SMILES: *OCCNCCCN1CCNCC1] | —NMeCH₂CH₂NMe₂ |
| 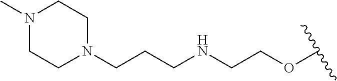 [SMILES: *OCCNCCCN1CCN(C)CC1] | 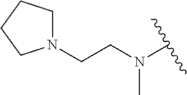 [SMILES: *N(C)CCN1CCCC1] |
| 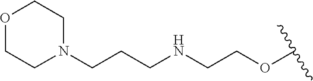 [SMILES: *OCCNCCCN1CCOCC1] | 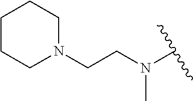 [SMILES: *N(C)CCN1CCCCC1] |
| —OCH₂CH₂CH₂NHCH₂CH₂OH | 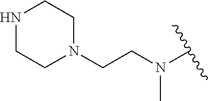 [SMILES: *N(C)CCN1CCNCC1] |
| —OCH₂CH₂CH₂NHCH₂CH₂OMe | 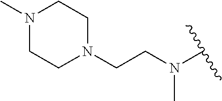 [SMILES: *N(C)CCN1CCN(C)CC1] |
| —OCH₂CH₂CH₂NHCH₂CH₂OEt | 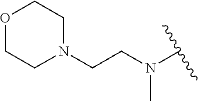 [SMILES: *N(C)CCN1CCOCC1] |
| —OCH₂CH₂CH₂NHCH₂CH₂NH₂ | —NMeCH₂CH₂CH₂OH |
| —OCH₂CH₂CH₂NHCH₂CH₂NHMe | —NMeCH₂CH₂CH₂OMe |
| —OCH₂CH₂CH₂NHCH₂CH₂NMe₂ | —NMeCH₂CH₂CH₂OEt |
| 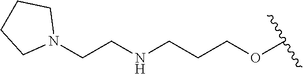 [SMILES: *OCCCNCCN1CCCC1] | —NMeCH₂CH₂CH₂NH₂ |
| 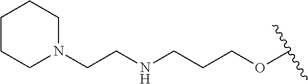 [SMILES: *OCCCNCCN1CCCCC1] | —NMeCH₂CH₂CH₂NHMe |
| 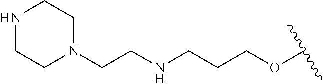 [SMILES: *OCCCNCCN1CCNCC1] | —NMeCH₂CH₂CH₂NMe₂ |
| 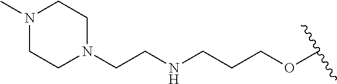 [SMILES: *OCCCNCCN1CCN(C)CC1] | 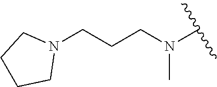 [SMILES: *N(C)CCCN1CCCC1] |
| 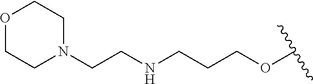 [SMILES: *OCCCNCCN1CCOCC1] | 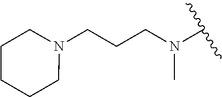 [SMILES: *N(C)CCCN1CCCCC1] |
| —OCH₂CH₂CH₂NHCH₂CH₂CH₂OH | 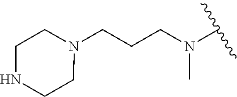 [SMILES: *N(C)CCCN1CCNCC1] |
| —OCH₂CH₂CH₂NHCH₂CH₂CH₂OMe | 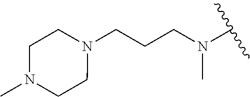 [SMILES: *N(C)CCCN1CCN(C)CC1] |
| —OCH₂CH₂CH₂NHCH₂CH₂CH₂OEt | 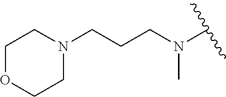 [SMILES: *N(C)CCCN1CCOCC1] |
| —OCH₂CH₂CH₂NHCH₂CH₂CH₂NH₂ | —NMeCH₂CH₂OCH₂CH₂OH |
| —OCH₂CH₂CH₂NHCH₂CH₂CH₂NHMe | —NMeCH₂CH₂OCH₂CH₂OMe |
| —OCH₂CH₂CH₂NHCH₂CH₂CH₂NMe₂ | —NMeCH₂CH₂OCH₂CH₂OEt |
| 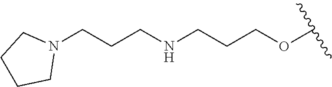 [SMILES: *OCCCNCCCN1CCCC1] | —NMeCH₂CH₂OCH₂CH₂NH₂ |
| 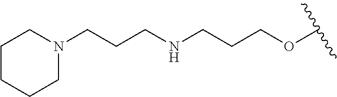 [SMILES: *OCCCNCCCN1CCCCC1] | —NMeCH₂CH₂OCH₂CH₂NHMe |
| 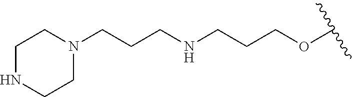 [SMILES: *OCCCNCCCN1CCNCC1] | —NMeCH₂CH₂OCH₂CH₂NMe₂ |
| 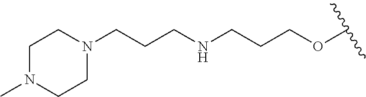 [SMILES: *OCCCNCCCN1CCN(C)CC1] | —O(C═O)OMe |
| 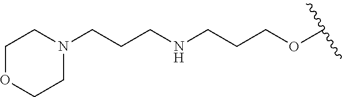 [SMILES: *OCCCNCCCN1CCOCC1] | —O(C═O)OEt |
| —OCH₂CH₂N(CH₂CH₂OH)₂ | —O(C═O)OnPr |
| —OCH₂CH₂N(CH₂CH₂OMe)₂ | —O(C═O)OiPr |
| —OCH₂CH₂N(CH₂CH₂OEt)₂ | —O(C═O)OcPr |
| —OCH₂CH₂N(CH₂CH₂CH₂OH)₂ | —O(C═O)OcBu |
| —OCH₂CH₂N(CH₂CH₂CH₂OMe)₂ | —O(C═O)OcPn |
| —OCH₂CH₂N(CH₂CH₂CH₂OEt)₂ | —O(C═O)OcHex |
| —OCH₂CH₂CH₂(HCH₂CH₂OH)₂ | —COOH |
| —OCH₂CH₂CH₂N(CH₂CH₂OMe)₂ | —CH₂(C═O)OH |
| —OCH₂CH₂CH₂N(CH₂CH₂OEt)₂ | —CH₂(C═O)OMe |
| —OCH₂CH₂CH₂N(CH₂CH₂CH₂OH)₂ | —CH₂(C═O)OEt |
| —OCH₂CH₂CH₂N(CH₂CH₂CH₂OMe)₂ | —CH₂(C═O)OnPr |
| —OCH₂CH₂CH₂N(CH₂CH₂CH₂OEt)₂ | —CH₂(C═O)OiPr |
| —OCH₂CH₂NMeCH₂CH₂OH | —CH₂(C═O)OcPr |
| —OCH₂CH₂NMeCH₂CH₂OMe | —CH₂(C═O)OcBu |
| —OCH₂CH₂NMeCH₂CH₂OEt | —CH₂(C═O)OcPn |
| —OCH₂CH₂NMeCH₂CH₂NH₂ | —CH₂(C═O)OcHex |
| —OCH₂CH₂NMeCH₂CH₂NHMe | —OCH₂(C═O)OH |
| —OCH₂CH₂NMeCH₂CH₂NMe₂ | —OCH₂(C═O)OMe |
| 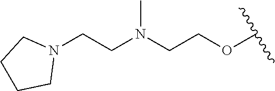 [SMILES: *OCCN(C)CCN1CCCC1] | —OCH₂(C═O)OEt |
| 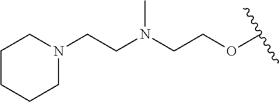 [SMILES: *OCCN(C)CCN1CCCCC1] | —OCH₂(C═O)OnPr |
| 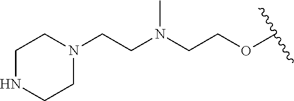 [SMILES: *OCCN(C)CCN1CCNCC1] | —OCH₂(C═O)OiPr |
| 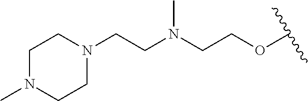 [SMILES: *OCCN(C)CCN1CCN(C)CC1] | —OCH₂(C═O)OcPr |
| 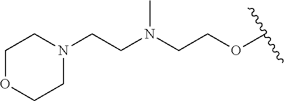 [SMILES: *OCCN(C)CCN1CCOCC1] | —OCH₂(C═O)OcBu |
| —OCH₂CH₂NMeCH₂CH₂CH₂OH | —OCH₂(C═O)OcPn |
| —OCH₂CH₂NMeCH₂CH₂CH₂OMe | —OCH₂(C═O)OcHex |
| —OCH₂CH₂NMeCH₂CH₂CH₂OEt | —OCH₂(C═O)OH |
| —OCH₂CH₂NMeCH₂CH₂CH₂NH₂ | —OCH₂(C═O)OMe |
| —OCH₂CH₂NMeCH₂CH₂CH₂NHMe | —OCH₂(C═O)OEt |
| —OCH₂CH₂NMeCH₂CH₂CH₂NMe₂ | —OCH₂(C═O)OnPr |
| 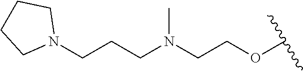 [SMILES: *OCCN(C)CCCN1CCCC1] | —OCH₂(C═O)OiPr |
| 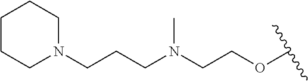 [SMILES: *OCCN(C)CCCN1CCCCC1] | —OCH₂(C═O)OcPr |
| 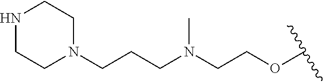 [SMILES: *OCCN(C)CCCN1CCNCC1] | —OCH₂(C═O)OcBu |
| 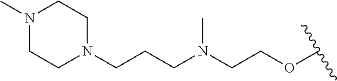 [SMILES: *OCCN(C)CCCN1CCN(C)CC1] | —OCH₂(C═O)OcPn |
| 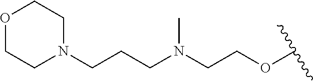 [SMILES: *OCCN(C)CCCN1CCOCC1] | —OCH₂(C═O)OcHex |
| —OCH₂CH₂CH₂NMeCH₂CH₂OH | —NHCH₂(C═O)OH |
| —OCH₂CH₂CH₂NMeCH₂CH₂OMe | —NHCH₂(C═O)OMe |
| —OCH₂CH₂CH₂NMeCH₂CH₂OEt | —NHCH₂(C═O)OEt |
| —OCH₂CH₂CH₂NMeCH₂CH₂NH₂ | —NHCH₂(C═O)OnPr |
| —OCH₂CH₂CH₂NMeCH₂CH₂NHMe | —NHCH₂(C═O)OiPr |
| —OCH₂CH₂CH₂NMeCH₂CH₂NMe₂ | —NHCH₂(C═O)OcPr |
| 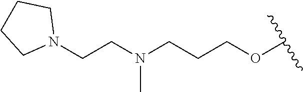 [SMILES: *OCCCN(C)CCN1CCCC1] | —NHCH₂(C═O)OcBu |
| 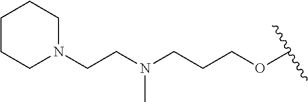 [SMILES: *OCCCN(C)CCN1CCCCC1] | —NHCH₂(C═O)OcPn |
| 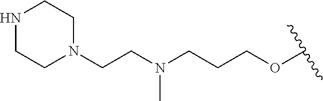 [SMILES: *OCCCN(C)CCN1CCNCC1] | —NHCH₂(C═O)OcHex |
| 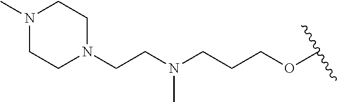 [SMILES: *OCCCN(C)CCN1CCN(C)CC1] | —NMeCH₂(C═O)OH |
| 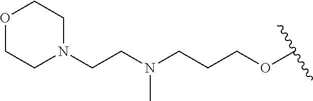 [SMILES: *OCCCN(C)CCN1CCOCC1] | —NH(CH₂(C═O)OH)₂ |
| —OCH₂CH₂CH₂NMeCH₂CH₂CH₂OH | —CH₂CH₂(C═O)OH |
| —OCH₂CH₂CH₂NMeCH₂CH₂CH₂OMe | —CH₂OCH₂(C═O)OH |
| —OCH₂CH₂CH₂NMeCH₂CH₂CH₂OEt | —OCH₂CH₂(C═O)OH |
| —OCH₂CH₂CH₂NMeCH₂CH₂CH₂NH₂ | —CH₂CH₂CH₂(C═O)OH |
| —OCH₂CH₂CH₂NMeCH₂CH₂CH₂NHMe | —CH₂CH₂OCH₂(C═O)OH |
| —OCH₂CH₂CH₂NMeCH₂CH₂CH₂NMe₂ | —CH₂OCH₂CH₂(C═O)OH |
| 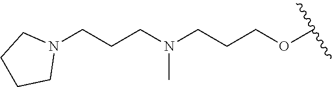 [SMILES: *OCCCN(C)CCCN1CCCC1] | —OCH₂CH₂CH₂(C═O)OH |
| 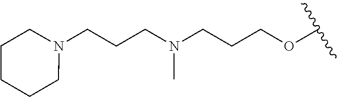 [SMILES: *OCCCN(C)CCCN1CCCCC1] | —OCH₂CH₂OCH₂(C═O)OH |
| 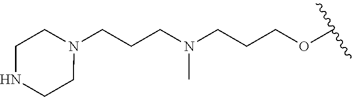 [SMILES: *OCCCN(C)CCCN1CCNCC1] | —CH₂CH₂CH₂CH₂(C═O)OH |
| 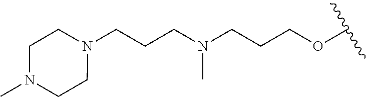 [SMILES: *OCCCN(C)CCCN1CCN(C)CC1] | —CH₂CH₂CH₂OCH₂(C═O)OH |
| 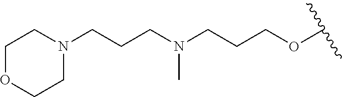 [SMILES: *OCCCN(C)CCCN1CCOCC1] | —CH₂CH₂OCH₂CH₂(C═O)OH |
| —CH₂OH | —CH₂OCH₂CH₂CH₂(C═O)OH |
| —CH₂OMe | —OCH₂CH₂CH₂CH₂(C═O)OH |
| —CH₂OEt | —CH₂OCH₂CH₂OCH₂(C═O)OH |
| —CH₂NH₂ | —OCH₂CH₂CH₂OCH₂(C═O)OH |
| —CH₂NHMe | —OCH₂CH₂OCH₂CH₂(C═O)OH |
| —CH₂NMe₂ | —CH₂OCH₂CH₂CH₂(C═O)OH |
| 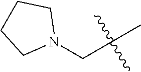 [SMILES: *CN1CCCC1] | —CH₂CH₂CH₂CH₂CH₂(C═O)OH |
| 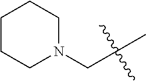 [SMILES: *CN1CCCCC1] | —CH₂CH₂CH₂CH₂OCH₂(C═O)OH |
| 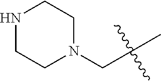 [SMILES: *CN1CCNCC1] | —CH₂CH₂CH₂OCH₂CH₂(C═O)OH |
| 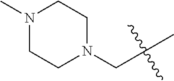 [SMILES: *CN1CCN(C)CC1] | —CH₂CH₂OCH₂CH₂CH₂(C═O)OH |
| 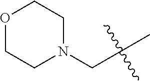 [SMILES: *CN1CCOCC1] | —CH₂OCH₂CH₂CH₂CH₂(C═O)OH |
| —CH₂CH₂OH | —OCH₂CH₂CH₂CH₂CH₂(C═O)OH |
| —CH₂CH₂OMe | —CH₂CH₂OCH₂CH₂OCH₂(C═O)OH |
| —CH₂CH₂OEt | —CH₂OCH₂CH₂CH₂OCH₂(C═O)OH |
| —CH₂CH₂NH₂ | —OCH₂CH₂CH₂CH₂OCH₂(C═O)OH |
| —CH₂CH₂NHMe | —CH₂OCH₂CH₂OCH₂CH₂(C═O)OH |
| —CH₂CH₂NMe₂ | —OCH₂CH₂CH₂OCH₂CH₂(C═O)OH |
| 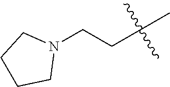 [SMILES: *CCN1CCCC1] | —OCH₂CH₂OCH₂CH₂CH₂(C═O)OH |
| 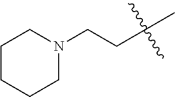 [SMILES: *CCN1CCCCC1] | —OCH₂CH₂OCH₂CH₂OCH₂(C═O)OH |
| 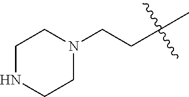 [SMILES: *CCN1CCNCC1] | —CH₂CH₂CH₂CH₂CH₂CH₂(C═O)OH |
| 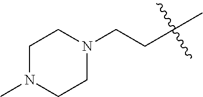 [SMILES: *CCN1CCN(C)CC1] | —CH₂CH₂CH₂CH₂CH₂OCH₂(C═O)OH |
| 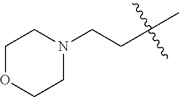 [SMILES: *CCN1CCOCC1] | —CH₂CH₂CH₂OCH₂CH₂OCH₂(C═O)OH |
| —CH₂CH₂CH₂OH | —CH₂OCH₂CH₂OCH₂CH₂OCH₂C(O)OH |
| —CH₂CH₂CH₂OMe | —OCH₂CH₂CH₂OCH₂CH₂OCH₂C(O)OH |
| —CH₂CH₂CH₂OEt | —CH₂CH₂CH₂CH₂OCH₂CH₂(C═O)OH |
| —CH₂CH₂CH₂NH₂ | —CH₂CH₂OCH₂CH₂OCH₂CH₂(C═O)OH |
| —CH₂CH₂CH₂NHMe | —CH₂OCH₂CH₂CH₂OCH₂CH₂(C═O)OH |
| —CH₂CH₂CH₂NMe₂ | —OCH₂CH₂CH₂CH₂OCH₂CH₂(C═O)OH |
| 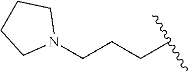 [SMILES: *CCCN1CCCC1] | —OCH₂CH₂OCH₂CH₂OCH₂CH₂(CO)OH |
| 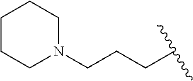 [SMILES: *CCCN1CCCCC1] | —CH₂CH₂CH₂OCH₂CH₂CH₂(C═O)OH |
| 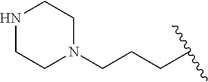 [SMILES: *CCCN1CCNCC1] | —CH₂OCH₂CH₂OCH₂CH₂CH₂(C═O)OH |
| 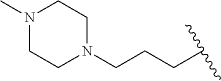 [SMILES: *CCCN1CCN(C)CC1] | —OCH₂CH₂CH₂OCH₂CH₂CH₂(C═O)OH |
| 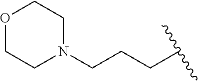 [SMILES: *CCCN1CCOCC1] | —CH₂CH₂OCH₂CH₂CH₂CH₂(C═O)OH |
| —CH₂CH₂CH₂CH₂OH | —OCH₂CH₂OCH₂CH₂CH₂CH₂(C═O)OH |
| —CH₂CH₂CH₂CH₂OMe | —CH₂OCH₂CH₂CH₂CH₂CH₂(C═O)OH |
| —CH₂CH₂CH₂CH₂OEt | —OCH₂CH₂CH₂CH₂CH₂CH₂(C═O)OH |
| —CH₂CH₂CH₂CH₂NH₂ | —NHCH₂CH₂(C═O)OH |
| —CH₂CH₂CH₂CH₂NHMe | —NMeCH₂CH₂(C═O)OH |
| —CH₂CH₂CH₂CH₂NMe₂ | —N(CH₂CH₂(C═O)OH)₂ |
| 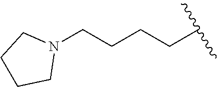 [SMILES: *CCCCN1CCCC1] | —NHCH₂CH₂OCH₂(C═O)OH |
| 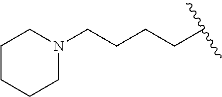 [SMILES: *CCCCN1CCCCC1] | —NMeCH₂CH₂OCH₂(C═O)OH |
| 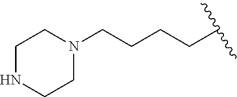 [SMILES: *CCCCN1CCNCC1] | —N(CH₂CH₂OCH₂(C═O)OH)₂ |
| 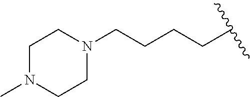 [SMILES: *CCCCN1CCN(C)CC1] | —NHCH₂CH₂CH₂CH₂(C═O)OH |
| 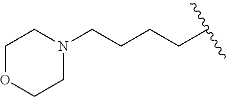 [SMILES: *CCCCN1CCOCC1] | —NMeCH₂CH₂CH₂CH₂(C═O)OH |
| —CH₂OCH₂CH₂OH | —N(CH₂CH₂CH₂CH₂(C═O)OH)₂ |
| —CH₂OCH₂CH₂OMe | —NHCH₂CH₂CH₂OCH₂(C═O)OH |
| —CH₂OCH₂CH₂OEt | —NMeCH₂CH₂CH₂OCH₂(C═O)OH |
| —CH₂OCH₂CH₂NH₂ | —N(CH₂CH₂CH₂OCH₂(C═O)OH)₂ |
| —CH₂OCH₂CH₂NHMe | —NHCH₂CH₂OCH₂CH₂(C═O)OH |
| —CH₂OCH₂CH₂NMe₂ | —NMeCH₂CH₂OCH₂CH₂(C═O)OH |
| 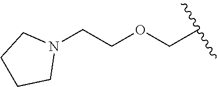 [SMILES: *COCCN1CCCC1] | —N(CH₂CH₂OCH₂CH₂(C═O)OH)₂ |
| 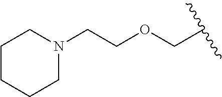 [SMILES: *COCCN1CCCCC1] | —NHCH₂CH₂CH₂CH₂CH₂(C═O)OH |
| 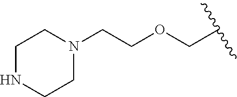 [SMILES: *COCCN1CCNCC1] | —NMeCH₂CH₂CH₂CH₂CH₂(C═O)OH |
| 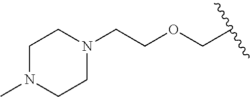 [SMILES: *COCCN1CCN(C)CC1] | —N(CH₂CH₂CH₂CH₂CH₂(C═O)OH)₂ |
| 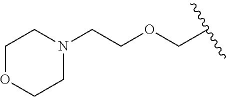 [SMILES: *COCCN1CCOCC1] | —NHCH₂CH₂CH₂CH₂OCH₂(C═O)OH |
| —CH₂NHCH₂CH₂OH | —NMeCH₂CH₂CH₂CH₂OCH₂(C═O)OH |
| —CH₂NHCH₂CH₂OMe | —N(CH₂CH₂CH₂CH₂OCH₂(C-O)OH)₂ |
| —CH₂NHCH₂CH₂OEt | —NHCH₂CH₂CH₂OCH₂CH₂(C═O)OH |
| —CH₂NHCH₂CH₂NH₂ | —NMeCH₂CH₂CH₂OCH₂CH₂(C═O)OH |
| —CH₂NHCH₂CH₂NHMe | —N(CH₂CH₂CH₂OCH₂CH₂(C═O)OH)₂ |
| —CH₂NHCH₂CH₂NMe₂ | —NHCH₂CH₂OCH₂CH₂CH₂(C═O)OH |
| 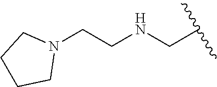 [SMILES: *CNCCN1CCCC1] | —NMeCH₂CH₂OCH₂CH₂CH₂(C═O)OH |
| 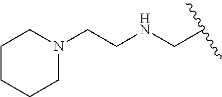 [SMILES: *CNCCN1CCCCC1] | —N(CH₂CH₂OCH₂CH₂CH₂(C═O)OH)₂ |
| 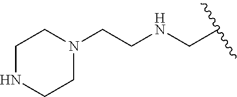 [SMILES: *CNCCN1CCNCC1] | —NHCH₂CH₂OCH₂CH₂OCH₂(C═O)OH |
| 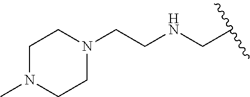 [SMILES: *CNCCN1CCN(C)CC1] | —NMeCH₂CH₂OCH₂CH₂OCH₂(C═O)OH |
| 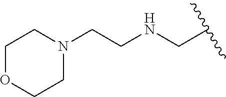 [SMILES: *CNCCN1CCOCC1] | —N(CH₂CH₂OCH₂CH₂OCH₂(C═O)OH)₂ |
| —CH₂N(CH₂CH₂OH)₂ | —NHCH₂CH₂CH₂OCH₂CH₂OCH₂CO₂H |
| —CH₂N(CH₂CH₂OMe)₂ | —NMeCH₂CH₂CH₂OCH₂CH₂OCH₂CO₂H |
| —CH₂N(CH₂CH₂OEt)₂ | —N(CH₂CH₂CH₂OCH₂CH₂OCH₂CO₂H)₂ |
| —CH₂NMeCH₂CH₂OH | —NHCH₂CH₂CH₂CH₂OCH₂CH₂CO₂H |
| —CH₂NMeCH₂CH₂OMe | —NMeCH₂CH₂CH₂CH₂OCH₂CH₂CO₂H |
| —CH₂NMeCH₂CH₂OEt | —N(CH₂CH₂CH₂CH₂OCH₂CH₂CO₂H)₂ |
| —CH₂NMeCH₂CH₂NH₂ | —NHCH₂CH₂OCH₂CH₂OCH₂CH₂CO₂H |
| —CH₂NMeCH₂CH₂NHMe | —NMeCH₂CH₂OCH₂CH₂OCH₂CH₂CO₂H |
| —CH₂NMeCH₂CH₂NMe₂ | —N(CH₂CH₂OCH₂CH₂OCH₂CH₂CO₂H)₂ |
| 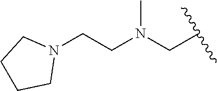 [SMILES: *CN(C)CCN1CCCC1] | —NHCH₂CH₂CH₂OCH₂CH₂CH₂CO₂H |
| 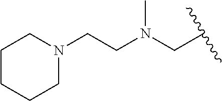 [SMILES: *CN(C)CCN1CCCCC1] | —NMeCH₂CH₂CH₂OCH₂CH₂CH₂CO₂H |
| 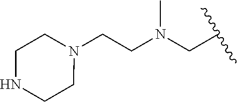 [SMILES: *CN(C)CCN1CCNCC1] | —N(CH₂CH₂CH₂OCH₂CH₂CH₂CO₂H)₂ |
| 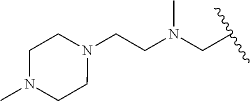 [SMILES: *CN(C)CCN1CCN(C)CC1] | —NHCH₂CH₂OCH₂CH₂CH₂CH₂CO₂H |
| 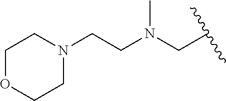 [SMILES: *CN(C)CCN1CCOCC1] | —NMeCH₂CH₂OCH₂CH₂CH₂CH₂CO₂H |
| —CH₂CH₂OCH₂CH₂OH | —N(CH₂CH₂OCH₂CH₂CH₂CH₂CO₂H)₂ |
| —CH₂CH₂OCH₂CH₂OMe | —NHCH₂CH₂CH₂CH₂CH₂CH₂CO₂H |
| —CH₂CH₂OCH₂CH₂OEt | —NMeCH₂CH₂CH₂CH₂CH₂CH₂CO₂H |
| —CH₂CH₂OCH₂CH₂NH₂ | —N(CH₂CH₂CH₂CH₂CH₂CH₂CO₂H)₂ |
| —CH₂CH₂OCH₂CH₂NHMe | |

In various embodiments, the compound of formula has the structure:

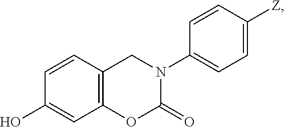

wherein Z is selected from the group consisting of

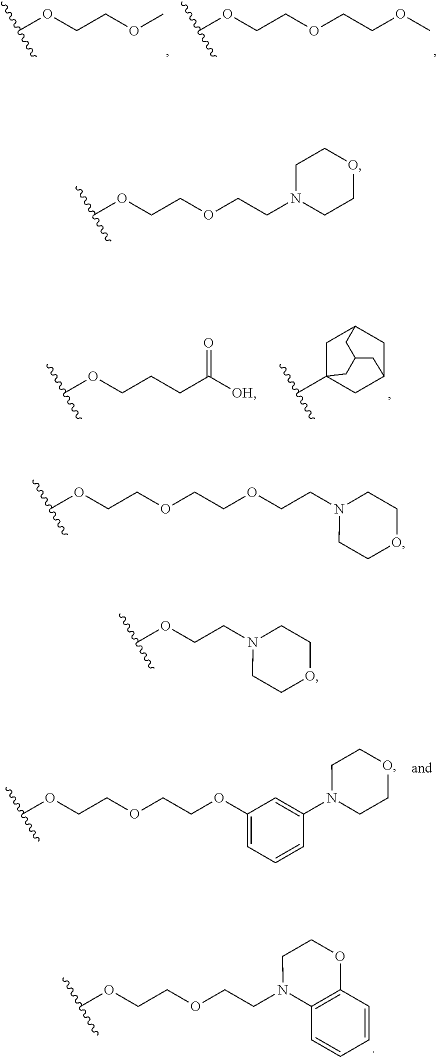

In various embodiments, the compound of formula (I) has the structure:

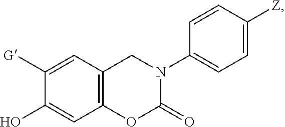

wherein Z is selected from the group consisting of

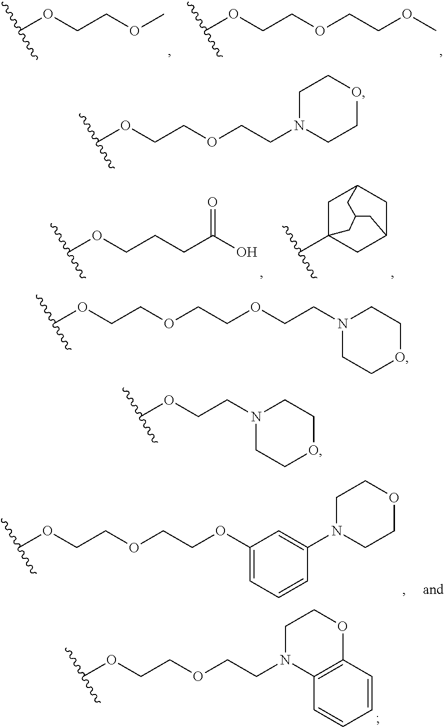

wherein G′ is halogen.

In various embodiments, G′ is F.

In various embodiments, the compound of formula (I) has a structure selected from the group consisting of

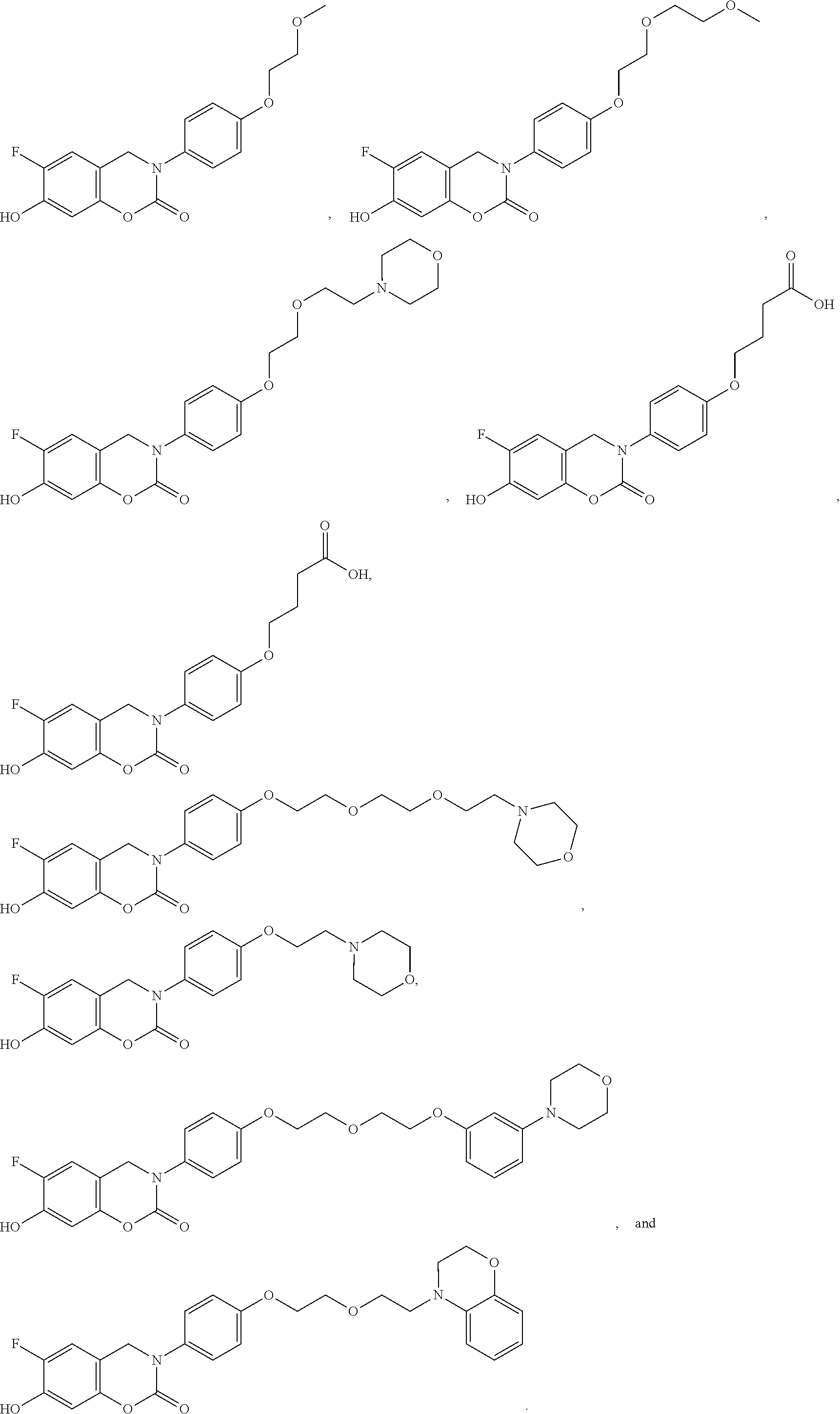

Table 2 lists activity data for a series of MIF antagonists obtained using the methods described in the Examples section herein.

TABLE 2
MIF Inhibition by Compounds of formula (Ia).

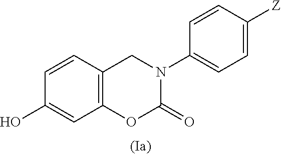

| Compound Name | Z | k_{d} (μM) |
|---|---|---|
| C1 | 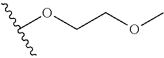 [SMILES: *OCCOC] | 0.006 ± 0.001 |
| C2 | 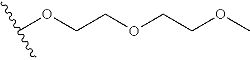 [SMILES: *OCCOCCOC] | 0.016 ± 0.005 |
| C3 | 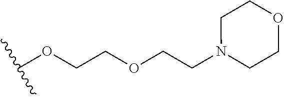 [SMILES: *OCCOCCN1CCOCC1] | 0.023 ± 0.013 |
| C4 | 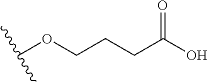 [SMILES: *OCCCC(=O)O] | 0.022 ± 0.008 |
| C6 | 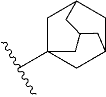 [SMILES: *C12CC3CC(CC(C3)C1)C2] | 0.828 ± 0.044 |
| C7 | 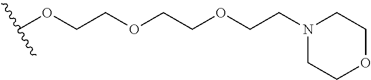 [SMILES: *OCCOCCOCCN1CCOCC1] | 0.023 ± 0.006 |
| C8 | 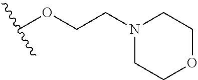 [SMILES: *OCCN1CCOCC1] | 0.012 ± 0.001 |
| C9 | 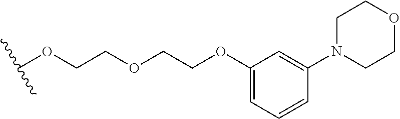 [SMILES: *OCCOCCOc1cccc(N2CCOCC2)c1] | 0.053 ± 0.007 |
| C10 | 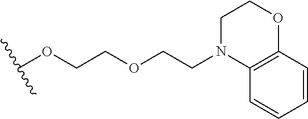 [SMILES: *OCCOCCN1CCOc2ccccc21] | 0.016 ± 0.001 |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

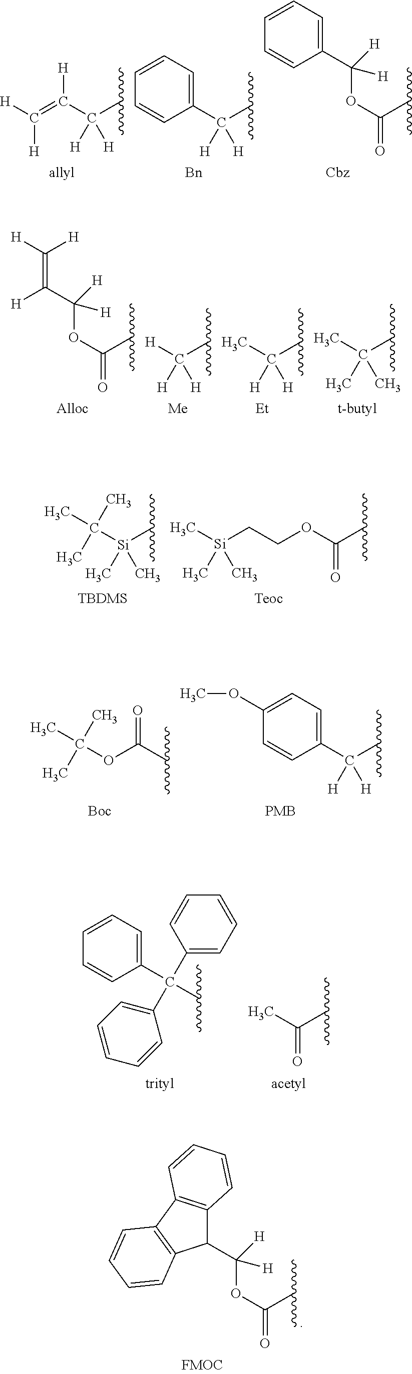

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

Compounds of formula (I) can inhibit the activity of macrophage migration inhibitory factor (MIF) and, thus, are useful in treating or ameliorating diseases and disorders associated with MIF activity. The disclosure provides a method of inhibiting macrophage migration inhibitory factor (MIF) activity in a subject using compounds of formula (I). The disclosure further provides a method of treating a disease or condition in which inhibition of macrophage migration inhibitory factor (MIF) activity in a subject is therapeutically beneficial. The disclosure further provides a method of treating an inflammatory disease or condition in a subject. The disclosure further provides a method of treating an autoimmune disease in a subject. The disclosure further provides a method of treating cancer in a subject. The disclosure further provides a method of treating a disease or condition associated with high MIF expression in a subject. The disclosure further provides a method of treating anemia of chronic disease in a subject. In certain embodiments, the method comprises administering to the subject an effective amount of a compound of the invention, or a pharmaceutically acceptable salt or solvate thereof.

In certain embodiments, the inflammatory disease or condition is selected from the group consisting of proliferative vascular disease, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Berger's disease, Retier's syndrome, and Hodgkin's disease.

In certain embodiments, the autoimmune disease is selected from the group consisting of multiple sclerosis, systemic lupus erythematosus, rheumatoid arthritis, graft versus host disease, autoimmune pulmonary inflammation, autoimmune encephalomyelitis, Guillain-Barre syndrome, autoimmune thyroiditis, insulin dependent diabetes mellitus, Crohn's disease, scleroderma, psoriasis, Sjögren's syndrome, and autoimmune inflammatory eye disease.

In certain embodiments, the cancer is a solid tumor or a hematological cancer.

In certain embodiments, the cancer is selected from the group consisting of prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head or neck, thyroid cancer, glioblastoma, sarcoma, bladder cancer, lymphoma, leukemia such as acute lymphoblastic leukemia (ALL), acute myelogenous leukemia (AML), chronic lymphocytic leukemia (CLL), chronic myelogenous leukemia (CML), diffuse large B-cell lymphoma (DLBCL), mantle cell lymphoma, non-Hodgkin's lymphoma (including relapsed non-Hodgkin's lymphoma, refractory non-Hodgkin's lymphoma and recurrent follicular non-Hodgkin's lymphoma), Hodgkin's lymphoma, and multiple myeloma.

In certain embodiments, the disease or condition associated with high MIF expression is selected from the group consisting of protozoal infection, fungal infection, bacterial infection, viral infection, anemia of chronic disease, asthma, and autism spectrum disorder (ASD).

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats any of the diseases or conditions described herein.

In certain embodiments, administering the compound(s) described herein to the subject allows for administering a lower dose of the additional therapeutic agent as compared to the dose of the additional therapeutic agent alone that is required to achieve similar results in treating a any of the diseases or conditions described herein in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating any of the diseases or conditions described herein. These additional therapeutic agents may comprise compounds that are commercially available or synthetically accessible to those skilled in the art. These additional therapeutic agents are known to treat or reduce the symptoms, of a condition or disease described herein.

In certain embodiments, the compounds described herein can be used in combination with radiation therapy. In other embodiments, the combination of administration of the compounds described herein and application of radiation therapy is more effective in treating or preventing any of the conditions or diseases described herein than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a condition or disease described herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a condition or disease described herein in the patient. An effective amount of the therapeutic compound necessary to achieve a therapeutic effect may vary according to factors such as the state of the disease or disorder in the patient; the age, sex, and weight of the patient; and the ability of the therapeutic compound to treat any of the diseases or conditions described herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient described herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 6%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% a-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 6%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.010% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, ₁%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a disease or condition described herein in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### Examples

Various embodiments of the present application can be better understood by reference to the following Examples which are offered by way of illustration. The scope of the present application is not limited to the Examples given herein.

Chemistry Methods. All reagents and solvents were obtained from commercial suppliers and used without further purification unless otherwise indicated. Column chromatography was carried out on silica gel (300-400 mesh). All reactions were monitored by thin-layer chromatography (TLC), and silica gel plates with fluorescence F-254 were used and visualized with UV light. All of the final compounds were purified to >95% purity, as determined by high-performance liquid chromatography (HPLC). HPLC analysis was performed on an Agilent 1260 Infinity II HPLC system with the use of an Agilent prep-C18 scalar reversed-column (4.6 mm×100 mm, 5 μm). The binary solvent system was 0.1% formic acid in water (A) and acetonitrile (B), and eluted in a gradient manner from 5% to 100% (A/B) in 15 minutes. The absorbance was detected at 254 nm, and the flow rate was 1.5 mL/min. ¹H NMR and ¹³C NMR spectra were recorded on an Agilent DD2 400 MHz NMR spectrometer. Coupling constants (J) are expressed in hertz (Hz). Spin multiplicities are described as s (singlet), br. s (broad singlet), t (triplet), q (quartet), dd (doublet of doublets) dt (doublet of triplets), td (triplet of doublets), and m (multiplet). Chemical shifts (6) are listed in parts per million (ppm) relative to internal standard tetramethylsilane (TMS) or solvent. Mass spectral (MS) data were acquired on an Advion Express mass spectrometer (Ithaca, NY, USA).

### Example 1: Synthesis of Compounds C3, C8, and C10

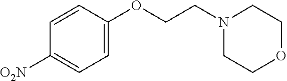 [SMILES: O=[N+]([O-])c1ccc(OCCN2CCOCC2)cc1]

4-(2-(4-Nitrophenoxy)ethyl)morpholine (S1a). 4-Nitrophenol (1.39 g, 10 mmol, 1.0 eq), 4-(2-chloroethyl)morpholine hydrochloride (2.23 g, 12 mmol, 1.2 eq) and potassium carbonate (3.45 g, 25 mmol, 2.5 eq) were suspended in DMF (30 mL) and stirred at 80° C. for 8 h. Then the mixture was concentrated in vacuo and the residue was extracted with water (40 mL) and dichloromethane (2×40 mL). The combined organic layer was concentrated in vacuo and the crude product was purified using silica gel chromatography with a methanol/dichloromethane gradient (0-5%) to yield the title compound as a yellow solid (2.15 g, yield 85%). ¹H NMR (400 MHz, CDCl₃) δ 8.19 (d, J=9.2 Hz, 2H), 6.96 (d, J=9.2 Hz, 2H), 4.19 (t, J=5.6 Hz, 2H), 3.73 (t, J=4.7 Hz, 4H), 2.83 (t, J=5.6 Hz, 2H), 2.58 (t, J=4.6 Hz, 4H). MS m/z (ESI): 253.2 [M+H]⁺.

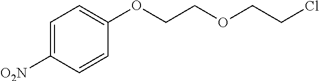 [SMILES: O=[N+]([O-])c1ccc(OCCOCCCl)cc1]

1-(2-(2-Chloroethoxy)ethoxy)-4-nitrobenzene (S1b′). S1b′ was prepared according to the procedure described for the synthesis of S1a using 4-nitrophenol (1.39 g, 10 mmol, 1.0 eq) and 1-chloro-2-(2-chloroethoxy)ethane (3.8 g, 15 mmol, 1.5 eq) with ethyl acetate/hexane gradient (0-5%). White solid, 1.92 g, yield 78%. ¹H NMR (400 MHz, CDCl₃) δ 8.20 (d, J=9.3 Hz, 2H), 6.99 (d, J=9.2 Hz, 2H), 4.29-4.18 (m, 2H), 3.99-3.88 (m, 2H), 3.84 (t, J=5.7 Hz, 2H), 3.66 (t, J=5.7 Hz, 2H).

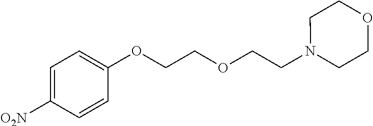 [SMILES: O=[N+]([O-])c1ccc(OCCOCCN2CCOCC2)cc1]

4-(2-(2-(4-Nitrophenoxy)ethoxy)ethyl)morpholine (S1b). 1-(2-(2-Chloroethoxy)ethoxy)-4-nitrobenzene (S1b′, 1.23 g, 5 mmol, 1.0 eq), morpholine (0.48 g, 5.5 mmol, 1.1 eq), potassium carbonate (1.38 g, 10 mmol, 2.0 eq), potassium iodide (0.83 g, 5 mmol, 1.0 eq) and Tetrabutylammonium iodide (0.37 g, 1 mmol, 0.2 eq) were suspended in DMF (30 mL) and stirred at 80° C. for 5 h. Then the mixture was concentrated in vacuo and the residue was extracted with water (40 mL) and dichloromethane (2×40 mL). The combined organic layer was concentrated in vacuo and the crude product was purified using silica gel chromatography with a methanol/dichloromethane gradient (0-5%) to yield the title compound as a yellow oil (0.84 g, yield 57%). ¹H NMR (400 MHz, CDCl₃) δ 8.18 (d, J=9.2 Hz, 2H), 6.95 (d, J=9.2 Hz, 2H), 4.29-4.11 (m, 2H), 3.92-3.79 (m, 2H), 3.69 (q, J=5.5, 5.0 Hz, 6H), 2.60 (t, J=5.7 Hz, 2H), 2.54-2.42 (m, 4H). MS m/z (ESI): 297.2 [M+H]⁺.

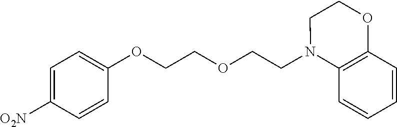 [SMILES: O=[N+]([O-])c1ccc(OCCOCCN2CCOc3ccccc32)cc1]

4-(2-(2-(4-Nitrophenoxy)ethoxy)ethyl)-3,4-dihydro-2H-benzo[b][1,4]oxazine (S1c). S1c was prepared according to the procedure described for the synthesis of Sib using 1-(2-(2-Chloroethoxy)ethoxy)-4-nitrobenzene (S1b′, 245 mg, 1 mmol, 1.0 eq) and 3,4-dihydro-2H-benzo[b][1,4]oxazine (142 mg, 1.05 mmol, 1.05 eq). Pale yellow solid, 41 mg, yield 12%. ¹H NMR (400 MHz, CDCl₃) δ 8.17 (d, J=9.2 Hz, 2H), 6.93 (d, J=9.2 Hz, 2H), 6.85-6.71 (m, 2H), 6.69-6.55 (m, 2H), 4.17 (t, J=4.5 Hz, 4H), 3.88-3.79 (m, 2H), 3.74 (t, J=5.7 Hz, 2H), 3.48 (t, J=5.7 Hz, 2H), 3.41 (t, J=4.4 Hz, 2H). MS m/z (ESI): 345.2 [M+H]⁺.

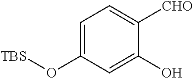

4-((tert-Butyldimethylsilyl)oxy)-2-hydroxybenzaldehyde (S2). 2,4-Dihydroxy benzaldehyde (2.76 g, 20 mmol, 1.0 eq) and imidazole (2.04 g, 30 mmol, 1.5 eq) were dissolved in dichloromethane (60 mL) and the mixture was cooled to 0° C., a solution of tert-butyldimethylsilyl chloride (3.3 g, 22 mmol, 1.1 eq) in dichloromethane (10 mL) was dropwise added and the mixture was stirred for 30 min. After the reaction completed, the mixture was washed with saturated aqueous NH₄Cl, water, brine, dried over MgSO₄, and the crude product was purified using silica gel chromatography with an ethyl acetate/hexanes gradient (0-10%) to afford the title compound as a clear oil (4.34 g, yield 86%). ¹H NMR (400 MHz, CDCl₃) δ 11.33 (s, 1H), 9.72 (s, 1H), 7.40 (d, J=8.5 Hz, 1H), 6.47 (dd, J=8.5, 2.2 Hz, 1H), 6.39 (d, J=2.0 Hz, 1H), 0.98 (s, 9H), 0.26 (s, 6H). MS m/z (ESI): 251.1 [M−H]⁻.

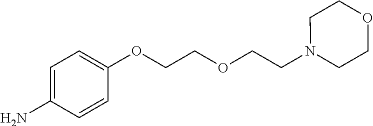 [SMILES: Nc1ccc(OCCOCCN2CCOCC2)cc1]

4-(2-(2-Morpholinoethoxy)ethoxy)aniline (S3a). 4-(2-(2-(4-Nitrophenoxy)ethoxy)ethyl)morpholine (Sib, 607 mg, 2.05 mmol) and palladium on activated carbon (10%, 50 mg) were suspended in methanol (25 mL), the mixture underwent 3 cycles of vacuum/filling with H₂ and stirred at 40° C. for 2 h. After the reaction was completed, the mixture was filtered, filtrate was used for the nest step without further purification. MS m/z (ESI): 267.2 [M+H]⁺.

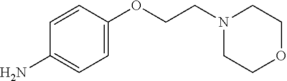 [SMILES: Nc1ccc(OCCN2CCOCC2)cc1]

4-(2-Morpholinoethoxy)aniline (S3b). 4-(2-(4-Nitrophenoxy)ethyl) morpholine (S1a 517 mg, 2.05 mmol) and palladium on activated carbon (10%, 50 mg) were suspended in methanol (25 mL), the mixture underwent 3 cycles of vacuum/filling with H₂ and stirred at 40° C. for 2 h. After the reaction was completed, the mixture was filtered, filtrate was used for the nest step without further purification. MS m/z (ESI): 223.2 [M+H]⁺.

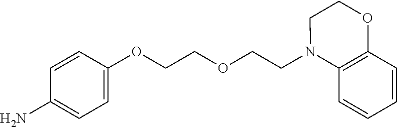 [SMILES: Nc1ccc(OCCOCCN2CCOc3ccccc32)cc1]

4-(2-(2-(2,3-Dihydro-4H-benzo[b][1,4]oxazin-4-yl)ethoxy)ethoxy) aniline (S3c). 4-(2-(2-(4-Nitrophenoxy)ethoxy)ethyl)-3,4-dihydro-2H-benzo[b][1,4] oxazine (S1c, 41 mg) and palladium on activated carbon (10%, 50 mg) were suspended in methanol (10 mL), the mixture underwent 3 cycles of vacuum/filling with H₂ and stirred at 40° C. for 1 h. After the reaction was completed, the mixture was filtered, filtrate was used for the nest step without further purification. MS m/z (ESI): 315.2 [M+H]⁺.

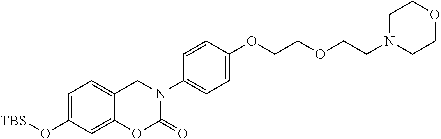

7-((tert-Butyldimethylsilyl)oxy)-3-(4-(2-(2-morpholinoethoxy) ethoxy)phenyl)-3,4-dihydro-2H-benzo[e][1,3]oxazin-2-one (S4a). 4-((tert-Butyldimethylsilyl) oxy)-2-hydroxybenzaldehyde (S2, 0.5 g, 2 mmol, 1.0 eq) was added to a solution of 4-(2-(2-morpholinoethoxy) ethoxy)aniline (S3a, 2 mmol, 1.0 eq) in methanol at rt. The reaction was completed after stirred for 1 h. Then the mixture was cooled to 0° C., sodium borohydride (0.23 g, 6 mmol, 3.0 eq) was added in portions at 0° C., the mixture was warmed to room temperature slowly and stirred for 3 h. The mixture was cooled to 0° C. and quenched with water and subsequently concentrated in vacuo, the residue was extracted with dichloromethane (2×30 mL) and brine (30 mL). The combined organic layer was dried over magnesium sulfate, filtered. Then 1,1′-Carbonyldiimidazole (0.65 g, 4 mmol, 2.0 eq) was added to the filtrate and stirred at 45° C. for 12 h. After the reaction was complete, the mixture was washed with saturated aqueous NH₄Cl, saturated aqueous NaHCO₃, brine, dried over magnesium sulfate, and the crude product was purified using silica gel chromatography with a methanol/dichloromethane gradient (0-5%) to yield the title compound as a white solid (0.29 g, yield 28%). ¹H NMR (400 MHz, CDCl₃) δ 7.29 (d, J=8.9 Hz, 2H), 6.95 (d, J=8.9 Hz, 3H), 6.66-6.58 (m, 2H), 4.72 (s, 2H), 4.13 (t, J=4.9 Hz, 2H), 3.82 (t, J=4.8 Hz, 2H), 3.76-3.66 (m, 6H), 2.62 (t, J=5.7 Hz, 2H), 2.52 (t, J=4.7 Hz, 4H), 0.98 (s, 9H), 0.21 (s, 6H). MS m/z (ESI): 529.3 [M+H]⁺.

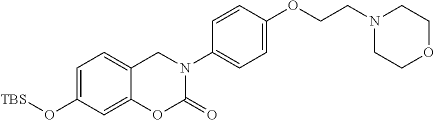

7-((tert-Butyldimethylsilyl)oxy)-3-(4-(2-morpholino-ethoxy) phenyl)-3,4-dihydro-2H-benzo[e] [1,3]oxazin-2-one (S4b). S4b was prepared according to the procedure described for the synthesis of S4a using 4-((tert-Butyldimethylsilyl) oxy)-2-hydroxybenzaldehyde (S2) and 4-(2-morpholinoethoxy)aniline (S3b), 0.37 g, yield 38.1%. ¹H NMR (400 MHz, CDCl₃) δ 7.28 (d, J=8.8 Hz, 2H), 6.94 (d, J=8.9 Hz, 3H), 6.65-6.56 (m, 2H), 4.71 (s, 2H), 4.10 (t, J=5.7 Hz, 2H), 3.72 (t, J=4.7 Hz, 4H), 2.79 (t, J=5.7 Hz, 2H), 2.56 (t, J=4.6 Hz, 4H), 0.97 (s, 9H), 0.20 (s, 6H). MS m/z (ESI): 485.3 [M+H]⁺.

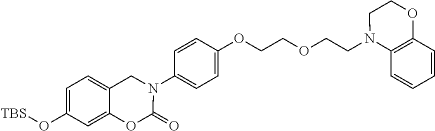

7-((tert-Butyldimethylsilyl)oxy)-3-(4-(2-(2-(2,3-dihydro-4H-benzo[b][1,4]oxazin-4-yl)ethoxy) ethoxy)phenyl)-3,4-dihydro-2H-benzo[e][1,3] oxazin-2-one (S4c). S4c was prepared according to the procedure described for the synthesis of S4a using 4-((tert-butyldimethylsilyl) oxy)-2-hydroxybenzaldehyde (S2) and 4-(2-(2-(2,3-dihydro-4H-benzo[b][1,4]oxazin-4-yl)ethoxy)ethoxy)aniline (S3c), 23 mg, yield 33.5%. MS m/z (ESI): 577.3 [M+H]⁺.

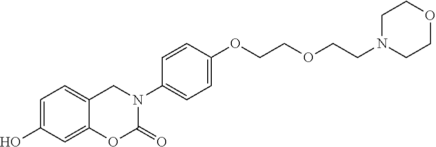 [SMILES: O=C1Oc2cc(O)ccc2CN1c1ccc(OCCOCCN2CCOCC2)cc1]

7-Hydroxy-3-(4-(2-(2-morpholinoethoxy)ethoxy) phenyl)-3,4-dihydro-2H-benzo[e][1,3]oxazin-2-one (C3). 1 M solution of tetrabutylammonium fluoride in THF (0.1 mL) was added to a solution of 7-((tert-Butyldimethylsilyl)oxy)-3-(4-(2-(2-morpholinoethoxy) ethoxy)phenyl)-3,4-dihydro-2H-benzo[e] [1,3]oxazin-2-one (S4a, 0.29 g, 0.56 mmol) in dry THF (10 mL) at 0° C. The mixture was stirred at room temperature for 1 h.

Then ethyl acetate (2×30 mL) and water (30 mL) were added for extraction, the combined organic layer was dried over magnesium sulfate. The crude product was purified using silica gel chromatography with a methanol/dichloromethane gradient (0-5%) to yield the title compound as a white solid (176 mg, yield 76%). ¹H NMR (400 MHz, DMSO-d₆) δ 9.74 (s, 1H), 7.33 (d, J=8.9 Hz, 2H), 7.04 (d, J=8.3 Hz, 1H), 6.96 (d, J=8.9 Hz, 2H), 6.56 (dd, J=8.3, 2.4 Hz, 1H), 6.45 (d, J=2.3 Hz, 1H), 4.69 (s, 2H), 4.09 (t, J=4.7 Hz, 2H), 3.71 (t, J=4.6 Hz, 2H), 3.55 (dt, J=14.8, 5.3 Hz, 6H), 2.46 (d, J=7.1 Hz, 2H), 2.38 (br.s, 4H). ¹³C NMR (101 MHz, DMSO-d₆) δ 158.22, 157.51, 150.55, 150.08, 135.48, 127.49, 127.02, 115.20, 112.03, 109.28, 102.56, 69.19, 67.79, 66.62, 58.04, 54.12, 50.37. MS m/z (ESI): 415.2 [M+H]⁺.

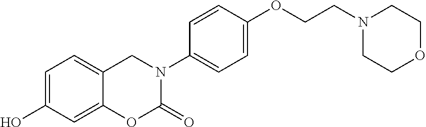 [SMILES: O=C1Oc2cc(O)ccc2CN1c1ccc(OCCN2CCOCC2)cc1]

7-Hydroxy-3-(4-(2-morpholinoethoxy)phenyl)-3,4-dihydro-2H-benzo[e][1,3]oxazin-2-one (C8). C8 was prepared according to the procedure described for the synthesis of C3 using 7-((tert-butyldimethylsilyl)oxy)-3-(4-(2-morpholinoethoxy) phenyl)-3,4-dihydro-2H-benzo[e] [1,3]oxazin-2-one (S4b). ¹H NMR (400 MHz, DMSO-d₆) δ 9.73 (s, 1H), 7.31 (d, J=8.8 Hz, 2H), 7.02 (d, J=8.4 Hz, 1H), 6.95 (d, J=8.9 Hz, 2H), 6.55 (dd, J=8.3, 2.3 Hz, 1H), 6.43 (d, J=2.3 Hz, 1H), 4.67 (s, 2H), 4.07 (t, J=5.7 Hz, 2H), 3.54 (t, J=4.7 Hz, 4H), 2.66 (t, J=5.7 Hz, 2H), 2.44 (t, J=4.6 Hz, 4H). ¹³C NMR (101 MHz, DMSO-d₆) δ 158.20, 157.47, 150.53, 150.08, 135.42, 127.47, 127.01, 115.20, 112.01, 109.27, 102.54, 66.61, 66.00, 57.39, 54.05, 50.35. MS m/z (ESI): 371.2 [M+H]⁺.

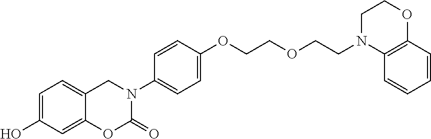 [SMILES: O=C1Oc2cc(O)ccc2CN1c1ccc(OCCOCCN2CCOc3ccccc32)cc1]

3-(4-(2-(2-(2,3-Dihydro-4H-benzo[b][1,4]oxazin-4-yl) ethoxy)ethoxy)phenyl)-7-hydroxy-3,4-dihydro-2H-benzo[e][1,3]oxazin-2-one (C10). C10 was prepared according to the procedure described for the synthesis of C3 using 7-((tert-butyldimethylsilyl)oxy)-3-(4-(2-(2-(2,3-dihydro-4H-benzo[b][1,4]oxazin-4-yl)ethoxy) ethoxy)phenyl)-3,4-dihydro-2H-benzo[e][1,3] oxazin-2-one (S4c). ¹H NMR (400 MHz, DMSO-d₆) δ 9.78 (s, 1H), 7.34 (d, J=8.9 Hz, 2H), 7.06 (d, J=8.4 Hz, 1H), 6.97 (d, J=9.0 Hz, 2H), 6.77-6.67 (m, 2H), 6.65 (dd, J=7.9, 1.4 Hz, 1H), 6.59 (dd, J=8.3, 2.4 Hz, 1H), 6.50 (dd, J=6.5, 1.8 Hz, 1H), 6.47 (d, J=2.4 Hz, 1H), 4.71 (s, 2H), 4.11 (t, J=5.7 Hz, 4H), 3.80-3.71 (m, 2H), 3.66 (t, J=5.7 Hz, 2H), 3.45 (t, J=5.7 Hz, 2H), 3.38 (t, J=4.4 Hz, 2H). ¹³C NMR (101 MHz, DMSO-d₆) δ 158.23, 157.52, 150.54, 150.29, 135.48, 127.47, 127.00, 121.76, 120.70, 117.37, 117.00, 116.29, 115.21, 112.41, 112.02, 109.27, 102.55, 69.38, 67.99, 67.88, 64.37, 50.35, 50.22, 47.79. MS m/z (ESI): 463.2 [M+H]⁺.

### Example 2: HPP and FP Assays

Tautomerase assay. Inhibition of the tautomerase activity of MIF was measured using 4-hydroxyphenyl pyruvic acid (HPP) as substrate, largely following previously reported protocols. HPP was dissolved in 0.5 M acetate buffer, pH 6.0 to a final concentration of 10 mM and incubated overnight at rt to allow equilibration of the keto and enol forms. MIF (6 μL) was premixed in 500 mM boric acid, pH 6.2 (142 μL) and transferred to a transparent U bottom 96-well plate (Falcon) to a final concentration of 50 nM MIF. For Ki determination, compounds were placed into wells (2 μL) at 6 different concentrations and incubated for 20 min until the assay was started by addition of HPP (50 μL) at two concentrations (1.0 and 2.5 mM). The negative control was MIF incubated with DMSO vehicle. MIF activity was monitored at 305 nm for formation of the borate-enol complex using an Infinite F500 plate reader (TECAN, Morrisville, NC) for 175 seconds. Calculation of initial velocities and the nonlinear regression analyses for the enzyme kinetics were repeated three times with the program Prism 6 (GraphPad, La Jolla, CA). Samples of Orita-13 and (R)-ISO-1 were purchased both from Alfa Aesar and Santa Cruz Biotechnology.

Determination of the affinities for A and B by FP assay. Determination of K_{d} values for the tracers followed previously reported protocols for other proteins. Experiments are carried out by quadruplicates in three independent experiments. In a flat black bottom 96 well plate (Corning), add to the third column 300 μL of 1.8 μM MIF in FP buffer (20 mM HEPES, 150 mM NaCl, 0.01% Tween-20, pH 7.4). From this column, using a multichannel pipette make serial dilutions (1:2) into the following wells to a total volume of 150 μL. First column contains 200 μL buffer and is used as a blank, while second column contains 150 μL buffer. 2 μL of DMSO are added to each well to keep 1% of DMSO, followed by the addition of 16.7 nM ligand A or B (48 μL) except first column (blank). Fluorescence polarization is measured at λ_{exc}=485±20 nm, λₑₘ=535±25 nm using an Infinite F500 plate reader until no FP variation was observed (typically 1 h). From the lowest and highest FP values (tracer free and tracer fully bound to MIF) we calculated the fraction of ligand bound to the protein to ligand total (L_{b}/Lₜ) for each concentration of MIF. We plotted these data to provide a typical saturation binding curve and using Prism 6 fit the results to the Hill equation.

In order to determine the existence of non-specific binding, if any, the same experiment with the same conditions is carried out by adding 2 μL of 1 mM NVS-2. No increase in FP values is detected in the presence of the inhibitor, therefore we assume the lack of non-specific binding.

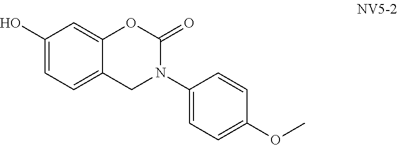 [SMILES: COc1ccc(N2Cc3ccc(O)cc3OC2=O)cc1]

Competitive FP assay. In order to calculate the K_{d} values of the unlabeled compounds, competitive assays were carried out in quadruplicates in three independent experiments. In a flatblack bottom 96 well plate (Corning) 140 μL of FP buffer are added to columns 3-12. First column contains 200 μL buffer (blank), while second column contains 150 μL FP buffer. 10 μL of 1.1 μM MIF are added to columns 3-12 followed by the addition of 2 μL of inhibitor in DMSO at 9 different concentrations. 2 μL of DMSO are added to columns 1-3. After 20 min of incubation at rt 48 μL of 16.7 nM ligand B are added to columns 2-12 and fluorescencepolarization was measured at λ_{exc}=485±20 nm, λₑₘ=535±25 nm for 1 h. Data are analyzed by a least-squares non-linear fit, generated using Prism 6 in order to determine the compound's IC₅₀. K_{d} values for each inhibitor are calculated using the following equation based on the IC₅₀, K_{d} of the tracer (K^{t}), total (Lₜ) and bound (L_{b}) tracer, as well as total MIF concentration (Pt). Samples of 4-IPP and Pontamine Sky Blue were purchased both from Tocris and Santa Cruz Biotechnology.

$$\text{?} = \frac{\text{?} {K}_{d}^{t}}{{P}_{t} \text{?} + \text{?} ( {P}_{t} - {L}_{t} + \text{?} - {K}_{d}^{t} )}$$

$$\text{?} \text{indicates text missing or illegible when filed}$$

### Example 3: Synthesis of Ligands A and B

NMR spectra were recorded on Agilent DD2 600 (600 MHz), DD2 500 (500 MHz) and DD2 400 (400 MHz) instruments. Column chromatography was carried out using CombiFlash over redisep column cartridges employing Merck silica gel (Kieselgel 60, 63-200 □m) and Grace C18 reversed-phase (40 □m). Pre-coated silica gel plates F-254 were used for thin-layer analytical chromatography. Mass determinations were performed using electrospray ionization on water Micromass ZQ (LC-MS) and on an Agilent Technologies 6890N (GC-MS). HRMS (ESI-TOF) analyses were performed on Waters Xevo QTOF equipped with Z-spray electrospray ionization source. The purity (≥95%) of all final synthesized compounds was determined by reverse phase HPLC, using a Waters 2487 dual λ absorbance detector with a Waters 1525 binary pump and a Phenomenex Luna 5p C18(2) 250×4.6 mm column. Samples were run at 1 mL/min using gradient mixtures of 5-100% of water with 0.10% trifluoroacetic acid (TFA) (A) and 10:1 acetonitrile:water with 0.10% TFA (B) for 22 min followed by 3 min at 100% B.

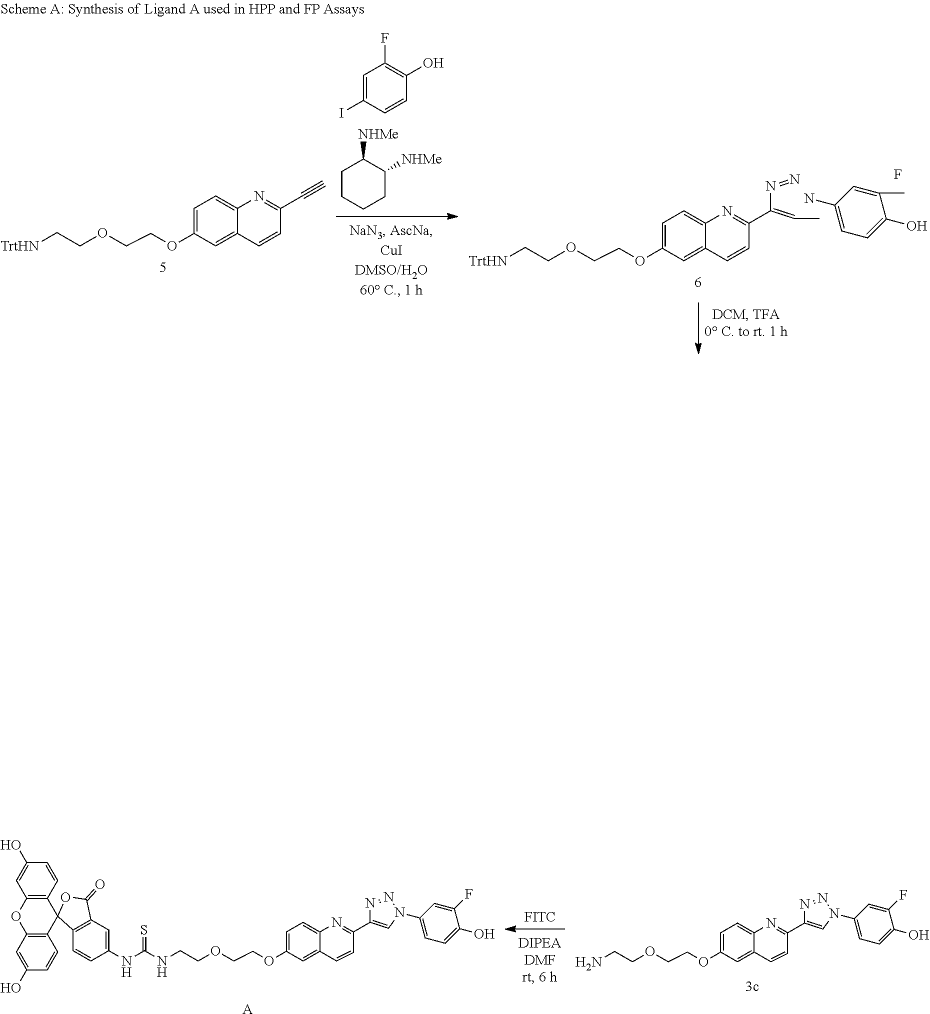

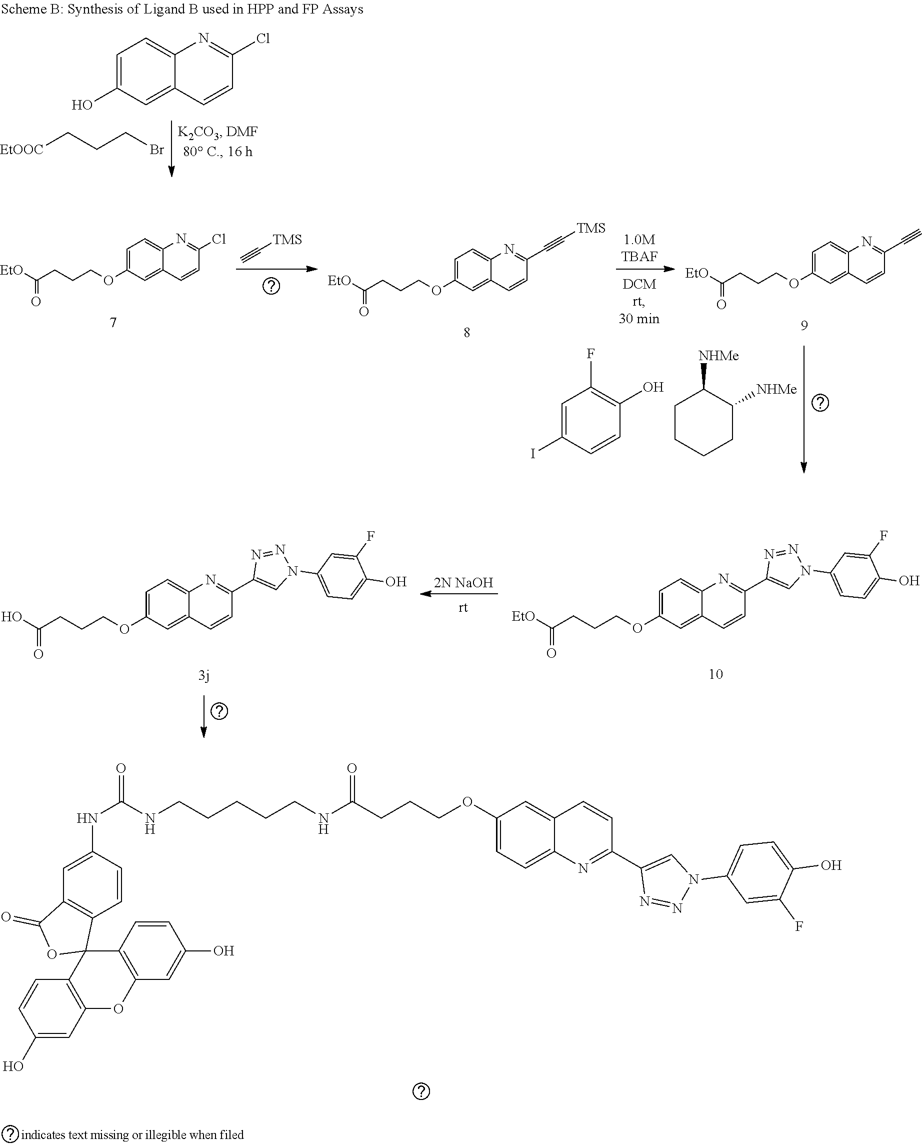

2-(2-((2-ethynylquinolin-6-yl)oxy)ethoxy)-N-tritylethan-1-amine (5). Synthesized as previously reported in Dziedzic, P.; Cisneros, J. A.; Robertson, M. J.; Hare, A. A.; Danford, N. E.; Baxter, R. H.; Jorgensen, W. L. J Am Chem Soc. 2015, 137, 2996-3003.

2-fluoro-4-(4-(6-(2-(2-(tritylamino)ethoxy)ethoxy)quinolin-2-yl)-1H-1,2,3-triazol-1-yl)phenol (6). 2-fluoro-4-iodopehnol (0.23 mmol) followed by trans-N,N′-dimethylcyclohexane-1,2-diamine (0.034 mmol), sodium ascorbate (0.023 mmol), copper iodide (0.023 mmol) and sodium azide (0.23 mmol) were added to DMSO (1.0 mL). The mixture was stirred at 70° C. for 2 h and then 5 (0.23 mmol) followed by H₂O (1.0 mL) were added to the reaction which was stirred overnight. The solution was then diluted with EtOAc and extracted with H₂O (×1) and brine (×1). The aqueous phase was washed with EtOAc, the organic phases were combined and dried with anh Na₂SO₄ and solvent evaporated. The crude product was purified by flash chromatography (hexanes/EtOAc). (27%) ¹H NMR (400 MHz, CDCl₃) δ 8.64 (s, 1H), 8.32 (d, J=8.6 Hz, 1H), 8.11 (d, J=8.6 Hz, 1H), 7.92 (d, J=9.2 Hz, 1H), 7.66 (dd, J=10.8, 2.5 Hz, 1H), 7.53-7.39 (m, 7H), 7.30 (dd, J=9.2, 2.7 Hz, 1H), 7.26-7.23 (m, 6H), 7.20-7.13 (m, 4H), 7.08 (d, J=2.8 Hz, 1H), 4.22 (t, J=4.7 Hz, 2H), 3.80 (t, J=4.7 Hz, 2H), 3.71 (t, J=5.3 Hz, 2H), 2.39 (t, J=5.3 Hz, 2H).

4-(4-(6-(2-(2-aminoethoxy)ethoxy)quinolin-2-yl)-1H-1,2,3-triazol-1-yl)-2-fluorophenol (3c). 6 (42 mg, 0.06 mmol) was dissolved in anh DCM (0.24 mL) and cooled to 0° C. Trifluoroacetic acid (0.16 mL) was added dropwise, the reaction warmed to rt and stirred for 1 h. Saturated NaHCO₃ solution was added to neutralize the reaction and extracted with DCM. Combined organic layers were dried over anh Na₂SO₄ and the final compound purified by flash chromatography (DCM/MeOH). (78%). ¹H NMR (400 MHz, DMSO-d₆) δ 10.56 (s, 1H), 9.31 (s, 1H), 8.39 (d, J=8.6 Hz, 1H), 8.24 (d, J=8.6 Hz, 1H), 8.02-7.79 (m, 4H), 7.76-7.69 (m, 1H), 7.52-7.41 (m, 2H), 7.17 (t, J=9.0 Hz, 1H), 4.36-4.25 (m, 2H), 3.93-3.86 (m, 2H), 3.71 (t, J=5.2 Hz, 2H), 3.04 (q, J=5.3 Hz, 2H). ¹³C NMR (151 MHz, DMSO-d₆) δ 156.4, 150.7 (d, J=242.8 Hz), 148.2, 147.5, 145.6 (d, J=11.9 Hz), 143.5, 136.1, 130.0, 128.5, 128.3 (d, J=8.8 Hz), 122.7, 121.4, 118.7, 118.2 (d, J=3.5 Hz), 116.9 (d, J=3.1 Hz), 109.3 (d, J=23.2 Hz), 106.7, 68.83, 67.4, 66.8, 38.6. HRMS (ESI): calcd. for [M+H]+(C₂₁H₂₀FN₅O₃) 410.1623, found 410.1625.

5-(3-(2-(2-((2-(1-(3-fluoro-4-hydroxyphenyl)-1H-1,2,3-triazol-4-yl)quinolin-6-yl)oxy)ethoxy)ethyl)thioureido)-2-(6-hydroxy-3-oxo-3H-xanthen-9-yl)benzoic acid (A). To a solution of 3c (30 mg) in 1 mL DMF under N₂, 0.7 mL DIPEA were added followed by the addition of 40 mg FITC and the reaction stirred for 6 h at rt protected from light. After solvent evaporation, final compound was purified by preparative HPLC. (80%). ¹H NMR (400 MHz, CD₃OD) δ 9.14 (s, 1H), 8.51-8.43 (m, 1H), 8.24-8.14 (m, 1H), 8.11-7.98 (m, 2H), 7.76-7.67 (m, 2H), 7.63-7.50 (m, 2H), 7.44 (s, 1H), 7.15 (t, J=8.8 Hz, 1H), 7.09-6.98 (m, 1H), 6.83-6.75 (m, 2H), 6.73-6.62 (m, 2H), 6.54 (d, J=9.1 Hz, 2H), 4.37 (s, 2H), 3.97 (s, 3H), 3.85 (s, 4H). ¹³C NMR (151 MHz, DMSO-d₆) δ 168.6, 159.5, 158.1, 156.5, 151.9, 151.9, 150.7 (d, J=242.8 Hz), 148.2, 147.5, 145.6 (d, J=11.4 Hz), 143.4, 141.5, 136.1, 132.9, 130.0, 129.3, 129.0, 128.5, 128.3 (d, J=9.2 Hz), 122.8, 121.4, 118.7, 118.2 (d, J=3.6 Hz), 116.9 (d, J=3.0 Hz), 6.4, 116.2, 112.6, 109.7, 109.4 (d, J=22.9 Hz), 106.8, 102.2, 83.0, 68.7, 68.6, 67.6, 41.5. HRMS (ESI): calcd. for [M+H]⁺ (C₄₂H₃₁FN₆O₈S) 799.1981, found 798.1992.

Ethyl 4-((2-chloroquinolin-6-yl)oxy)butanoate (7). 2-chloroquinolin-6-ol (200 mg, 1.1 mmol) was added to a pressure vial. Next, DMF (5 mL), ethyl 4-bromobutanoate (0.24 mL, 1.68 mmol) and K₂CO₃ (306 mg, 2.2 mmol) were added and the solution was stirred at 80° C. for 16 h. The reaction mixture was cooled to rt, diluted with EtOAc and washed with H₂O (×1) and brine (×3). The organic phase was dried over anh Na₂SO₄ and concentrated under vacuum. Intermediate 7 was used in the next step without further purification. (84%). ¹H NMR (400 MHz, CDCl₃) δ 7.97 (d, J=8.6 Hz, 1H), 7.90 (d, J=9.2 Hz, 1H), 7.39-7.30 (m, 2H), 7.06 (d, J=2.7 Hz, 1H), 4.18-4.09 (m, 4H), 2.55 (t, J=7.2 Hz, 2H), 2.22-2.13 (m, 2H), 1.25 (t, J=7.1 Hz, 3H).

Ethyl 4-((2-((trimethylsilyl)ethynyl)quinolin-6-yl)oxy)butanoate (8). Dry THF (4 mL), 7 (270 mg, 0.92 mmol), ethynyltrimethylsilane (1.4 mmol), Pd(PPh₃)₂Cl₂ (0.046 mmol), CuI (0.046 mmol) and dry Et₃N (3.7 mmol) were added to a pressure vial. The reaction mixture was stirred at 60° C. for 16 h. The crude reaction mixture was filtered through celite pad and washed with EtOAc. After solvent evaporation, desired compound was purified by flash chromatography (hexanes/EtOAc). (76%). ¹H NMR (400 MHz, CDCl₃) δ 7.97 (t, J=8.4 Hz, 2H), 7.48 (d, J=8.4 Hz, 1H), 7.34 (dd, J=9.3, 2.7 Hz, 1H), 7.02 (d, J=2.7 Hz, 1H), 4.19-4.09 (m, 4H), 2.55 (t, J=7.2 Hz, 2H), 2.22-2.13 (m, 2H), 1.25 (t, J=7.1 Hz, 3H), 0.29 (s, 9H).

Ethyl 4-((2-ethynylquinolin-6-yl)oxy)butanoate (9). 8 (250 mg, 0.7 mmol) was dissolved in anh DCM (7 mL). Next, 1.0 M TBAF in hexanes (0.84 mL) was added dropwise under N₂ and the reaction mixture was stirred at rt for 1 min. Upon completion 10 mL of 10% citric acid were added and the mixture stirred for 30 min. After washing with H₂O, the organic phase was dried over anh Na₂SO₄, concentrated under vacuum and the product used in the next step without further purification. (92%). ¹H NMR (400 MHz, CDCl₃) δ 7.99 (d, J=2.3 Hz, 1H), 7.97 (d, J=3.2 Hz, 1H), 7.49 (d, J=8.5 Hz, 1H), 7.35 (dd, J=9.3, 2.7 Hz, 1H), 7.03 (d, J=2.7 Hz, 1H), 4.21-4.07 (m, 4H), 3.19 (s, 1H), 2.55 (t, J=7.2 Hz, 2H), 2.25-2.12 (m, 2H), 1.26 (t, J=7.1 Hz, 4H).

Ethyl 4-((2-(1-(3-fluoro-4-hydroxyphenyl)-1H-1,2,3-triazol-4-yl)quinolin-6-yl)oxy)butanoate (10). Following the synthesis of 6. Purified by flash chromatography (DCM/EtOAc) (57%). ¹H NMR (400 MHz, CDCl₃) δ 8.66 (s, 1H), 8.33 (d, J=8.6 Hz, 1H), 8.15 (d, J=8.6 Hz, 1H), 7.97 (d, J=9.2 Hz, 1H), 7.67 (dd, J=10.7, 2.5 Hz, 1H), 7.53-7.47 (m, 1H), 7.37 (dd, J=9.2, 2.7 Hz, 1H), 7.18 (t, J=8.9 Hz, 1H), 7.10 (d, J=2.8 Hz, 1H), 4.20-4.08 (m, 4H), 2.58 (t, J=7.2 Hz, 2H), 2.24-2.17 (m, 3H), 1.27 (t, J=7.1 Hz, 3H).

4-((2-(1-(3-fluoro-4-hydroxyphenyl)-1H-1,2,3-triazol-4-yl)quinolin-6-yl)oxy)butanoic acid (3j). Ethyl ester 10 (25 mg, 0.06 mmol) was dissolved in dioxane (3.5 mL) and 2 N NaOH solution (1.6 mL) was added to the solution which was then stirred at rt. Upon completion, solvent was evaporated, the mixture diluted in H₂O and the pH was adjusted to around 3-4 with 1 N HCl solution. After cooling at 4° C. the crude was filtered and dried to give the carboxylic acid 11 without further purification. (85%). ¹H NMR (400 MHz, DMSO-d₆) δ 9.26 (s, 1H), 8.35 (d, J=8.6 Hz, 1H), 8.19 (d, J=8.6 Hz, 1H), 7.99-7.84 (m, 2H), 7.67 (ddd, J=9.1, 2.6, 1.3 Hz, 1H), 7.41 (d, J=7.8 Hz, 2H), 7.16 (t, J=9.0 Hz, 1H), 4.13 (t, J=6.5 Hz, 2H), 2.39 (t, J=7.3 Hz, 2H), 2.01 (quint, J=6.9 Hz, 2H). ¹³C NMR (101 MHz, DMSO-d₆) δ 174.4, 156.6, 151.8 (d, J=262.5 Hz), 148.2, 147.4, 146.5 (d, J=14.0 Hz), 143.4, 136.0, 130.0, 128.6, 128.3 (d, J=8.5 Hz), 122.8, 121.3, 118.6, 118.3 (d, J=3.9 Hz), 116.8 (d, J=2.4 Hz), 109.3 (d, J=20.0 Hz), 106.7, 67.3, 30.7, 24.4. HRMS (ESI): calc for [M+H]⁺ (C₂₁H₁₇FN₄O₄) 409.1307. found 409.1466.

5-(3-(5-(4-((2-(1-(3-fluoro-4-hydroxyphenyl)-1H-1,2,3-triazol-4-yl)quinolin-6-yl)oxy)butanamido)pentyl)thioureido)-2-(6-hydroxy-3-oxo-3H-xanthen-9-yl)benzoic acid (B). To an ice-cooled solution of 11 (50 mg, 0.12 mmol) in anh DMF (2 mL), Et₃N (20 μL), HOBt (19 mg, 0.15 mmol) and PyBOP (76 mg, 0.15 mmol) were added. The mixture was stirred at 0° C. for 20 min followed by the addition of 72 mg (0.15 mmol) of fluorescein-cadaverine. The reaction was warmed to rt and stirred overnight. Upon completion of the reaction, the mixture was diluted in EtOAc and washed with saturated NH₄Cl solution. The organic phase is dried over anh Na₂SO₄, solvent evaporated and the desired compound purified by C18 reverse phase chromatography (H₂O/ACN). (48%). ¹H NMR (500 MHz, DMSO-d₆) δ 10.77 (s, 1H), 10.57 (s, 1H), 10.19 (s, 1H), 9.29 (s, 1H), 8.63 (s, 1H), 8.43 (s, 1H), 8.38 (d, J=8.6 Hz, 1H), 8.21 (d, J=8.5 Hz, 1H), 7.97-7.89 (m, 3H), 7.87-7.82 (m, 1H), 7.73-7.70 (m, 1H), 7.45-7.40 (m, 2H), 7.21 (t, J=9.0 Hz, 1H), 7.15 (d, J=8.3 Hz, 1H), 6.70 (d, J=2.2 Hz, 2H), 6.62-6.51 (m, 4H), 4.13 (t, J=6.4 Hz, 2H), 3.50-3.44 (m, 2H), 3.08 (q, J=6.4 Hz, 2H), 2.31 (t, J=7.3 Hz, 2H), 2.06-2.00 (m, 2H), 1.59-1.53 (m, 2H), 1.47-1.41 (m, 2H), 1.39-1.32 (m, 2H). ¹³C NMR (151 MHz, DMSO-d₆) δ 171.3, 168.6, 166.2, 159.5, 156.6, 151.9, 150.7 (d, J=242.7 Hz), 149.7, 148.2, 147.4, 146.6, 145.6 (d, J=11.9 Hz), 143.4, 141.8, 136.0, 130.0, 129.0, 129.0, 128.6, 128.3 (d, J=9.1 Hz), 126.4, 123.9, 122.8, 121.4, 118.6, 118.2 (d, J=3.4 Hz), 116.8 (d, J=3.3 Hz), 112.6, 109.7, 109.3 (d, J=23.2 Hz). 106.7, 102.2, 83.0, 67.5, 43.5, 38.4, 31.7, 28.8, 28.0, 24.9, 23.9. HRMS (ESI): calcd. for [M+H]⁺ (C₄₇H₄₀FN₇O₈S) 882.2716, found 882.2645.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following e embodiments are provided, the numbering of which is not to be construed as designating levels of importance:

Embodiment 1 provides a compound of formula (I), or a salt, enantiomer, tautomer, or solvate thereof:

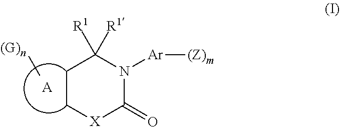

wherein:
R¹ and R^{1′} are each independently H, C₁₋₃ alkyl, or halogen;
X is CH₂, O, or S;
A is a fused 5-membered heteroaryl ring or a fused 6-membered aryl or heteroaryl ring;
each G is independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, and combinations thereof;
Ar is a 5-membered heteroaryl or a 6-membered aryl or heteroaryl ring;
each Z is a substituent on Ar independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, —(OCH₂CH₂)ₚ-4-10 membered heterocyclyl, —O(CH₂CH₂O)ₚ-4-10 membered heterocyclyl, —(OCH₂CH₂)ₚ-4-10 membered heteroaryl, —O(CH₂CH₂O)ₚ-4-10 membered heteroaryl, —O(CH₂CH₂O)ₚ—C₁₋₄ alkyl, and combinations thereof;
each R and R′ is independently H or C₁₋₁₀ hydrocarbyl;
n is an integer from 1 to 4;
m is an integer from 1 to 5; and
p is an integer from 1 to 5;
with the proviso that if Ar is phenyl; A is a fused 6-membered aryl ring having the structure

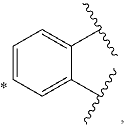 [SMILES: *c1ccccc1*]

substituted at the position marked with * by hydrogen, hydroxy, C₁₋₅ alkoxy, C₁₋₅ haloalkyl, or C₁₋₅ haloalkoxy; and m is 1; then Z is not OH, C₁₋₅ alkyl, C₁₋₅ alkoxy, C₁₋₅ haloalkyl, C₁₋₅ haloalkoxy, OCF₃, phenyl, or halogen.

Embodiment 2 provides the compound of embodiment 1, having the structure

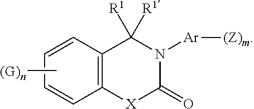

Embodiment 3 provides the compound of any one of embodiments 1-2, wherein X is O.

Embodiment 4 provides the compound of any one of embodiments 1-3, wherein R¹ and R^{1′} are H.

Embodiment 5 provides the compound of any one of embodiments 1-4, having the structure:

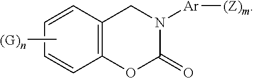

Embodiment 6 provides the compound of any one of embodiments 1-5, having the structure:

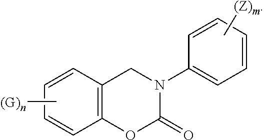

Embodiment 7 provides the compound of any one of embodiments 1-6, having the structure:

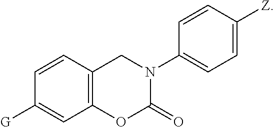

Embodiment 8 provides the compound of any one of embodiments 1-7, having the structure:

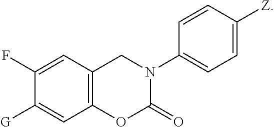

Embodiment 9 provides the compound of any one of embodiments 1-8, wherein G is OH.

Embodiment 10 provides the compound of any one of embodiments 1-9, wherein Z is selected from the group consisting of —(OCH₂CH₂)ₚ-4-10 membered non-aromatic heterocyclyl, —O(CH₂CH₂O)ₚ-4-10 membered non-aromatic heterocyclyl, —(OCH₂CH₂)ₚ-4-10 membered heteroaryl, —O(CH₂CH₂O)ₚ-4-10 membered heteroaryl, and —O(CH₂CH₂O)ₚ—C₁₋₄ alkyl.

Embodiment 11 provides the compound of any one of embodiments 1-10, wherein Z is selected from the group consisting of

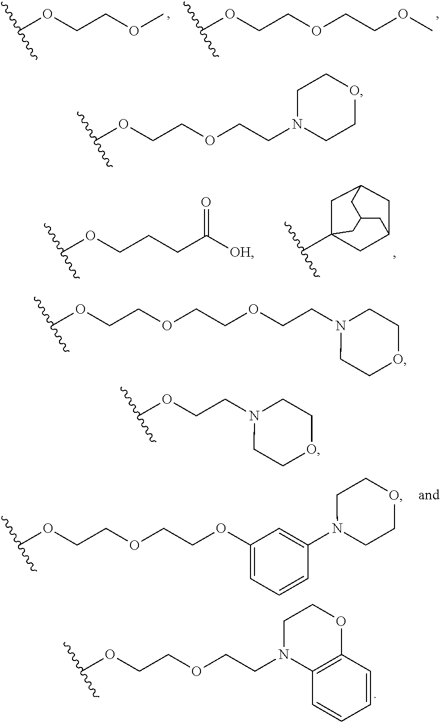

Embodiment 12 provides the compound of any one of embodiments 1-11, which is selected from the group consisting of:

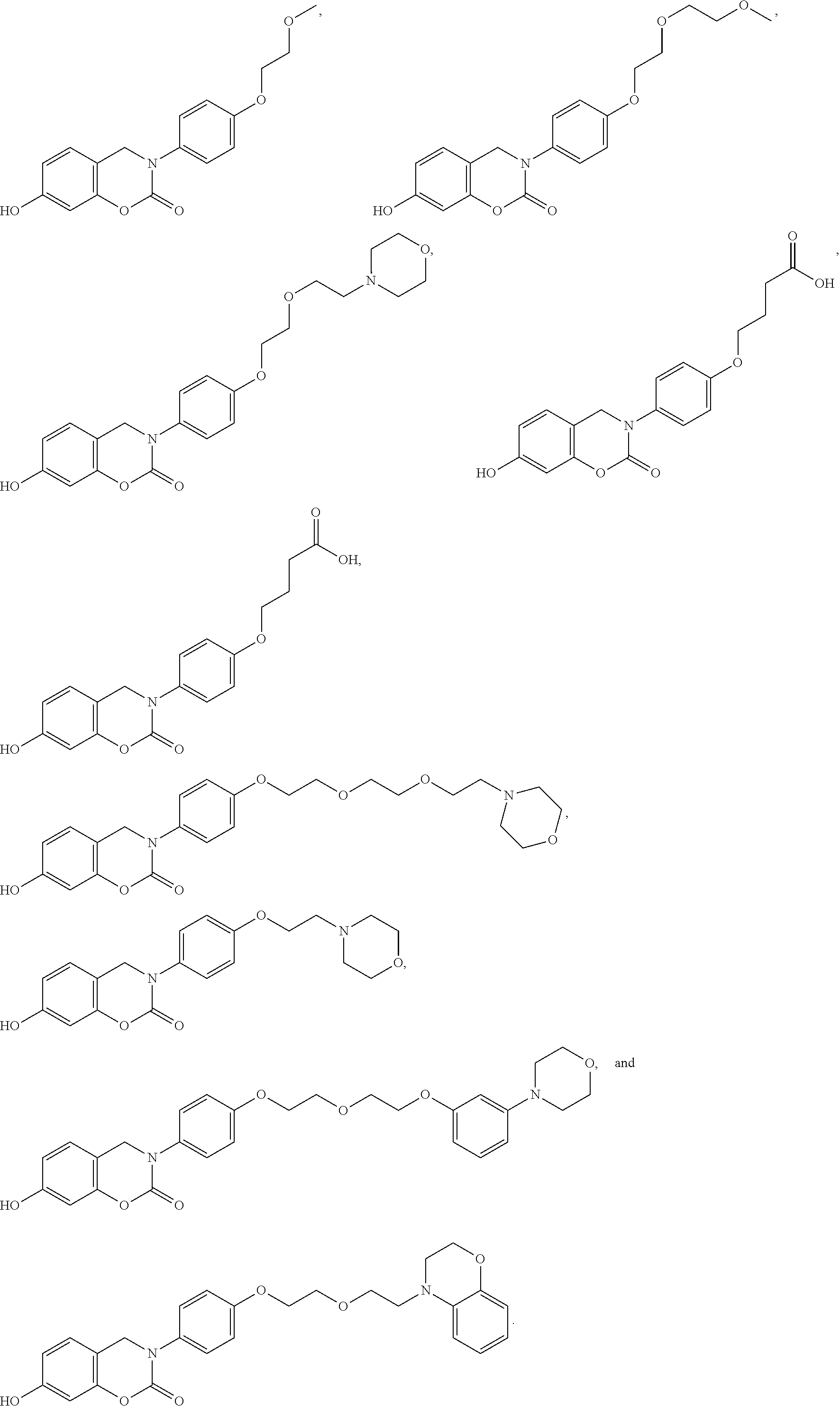

Embodiment 13 provides the compound of any one of embodiments 1-12, wherein Z is selected from the group consisting of

| | |
|---|---|
| | —CH₂CH₂OCH₂CH₂NMe₂ |
| —OH | 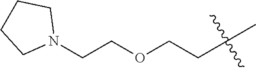 [SMILES: *CCOCCN1CCCC1] |
| —OEt | 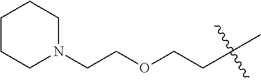 [SMILES: *CCOCCN1CCCCC1] |
| —OPr | 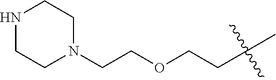 [SMILES: *CCOCCN1CCNCC1] |
| —OiPr | 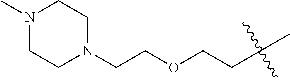 [SMILES: *CCOCCN1CCN(C)CC1] |
| —OcPr | 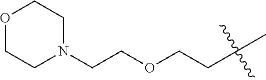 [SMILES: *CCOCCN1CCOCC1] |
| —OcBu | —CH₂CH₂NHCH₂CH₂OH |
| —OcPn | —CH₂CH₂NHCH₂CH₂OMe |
| —OcHex | —CH₂CH₂NHCH₂CH₂OEt |
| —OCH₂CH₂OH | —CH₂CH₂NHCH₂CH₂NH₂ |
| —OCH₂CH₂OMe | —CH₂CH₂NHCH₂CH₂NHMe |
| —OCH₂CH₂OEt | —CH₂CH₂NHCH₂CH₂NMe₂ |
| —OCH₂CH₂NH₂ | 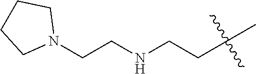 [SMILES: *CCNCCN1CCCC1] |
| —OCH₂CH₂NHMe | 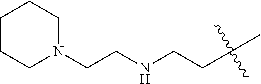 [SMILES: *CCNCCN1CCCCC1] |
| —OCH₂CH₂NMe₂ | 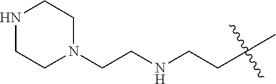 [SMILES: *CCNCCN1CCNCC1] |
| 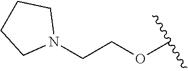 [SMILES: *OCCN1CCCC1] | 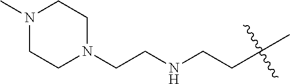 [SMILES: *CCNCCN1CCN(C)CC1] |
| 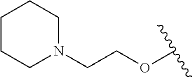 [SMILES: *OCCN1CCCCC1] | 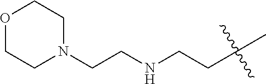 [SMILES: *CCNCCN1CCOCC1] |
| 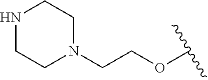 [SMILES: *OCCN1CCNCC1] | —CH₂CH₂N(CH₂CH₂OH)₂ |
| 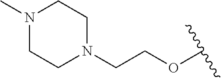 [SMILES: *OCCN1CCN(C)CC1] | —CH₂CH₂N(CH₂CH₂OMe)₂ |
| 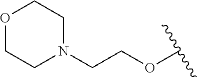 [SMILES: *OCCN1CCOCC1] | —CH₂CH₂N(CH₂CH₂OEt)₂ |
| —OCH₂CH₂CH₂OH | —CH₂CH₂NMeCH₂CH₂OH |
| —OCH₂CH₂CH₂OMe | —CH₂CH₂NMeCH₂CH₂OMe |
| —OCH₂CH₂CH₂OEt | —CH₂CH₂NMeCH₂CH₂OEt |
| —OCH₂CH₂CH₂NH₂ | —CH₂CH₂NMeCH₂CH₂NH₂ |
| —OCH₂CH₂CH₂NHMe | —CH₂CH₂NMeCH₂CH₂NHMe |
| —OCH₂CH₂CH₂NMe₂ | —CH₂CH₂NMeCH₂CH₂NMe₂ |
| 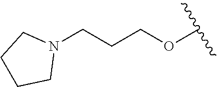 [SMILES: *OCCCN1CCCC1] | 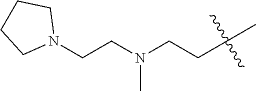 [SMILES: *CCN(C)CCN1CCCC1] |
| 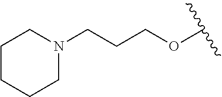 [SMILES: *OCCCN1CCCCC1] | 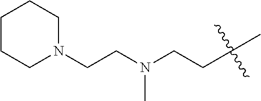 [SMILES: *CCN(C)CCN1CCCCC1] |
| 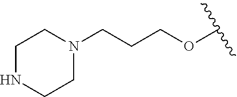 [SMILES: *OCCCN1CCNCC1] | 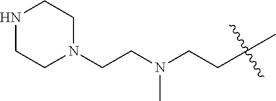 [SMILES: *CCN(C)CCN1CCNCC1] |
| 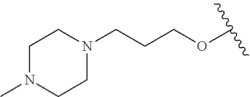 [SMILES: *OCCCN1CCN(C)CC1] | 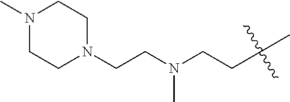 [SMILES: *CCN(C)CCN1CCN(C)CC1] |
| 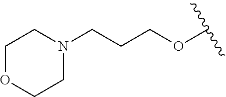 [SMILES: *OCCCN1CCOCC1] | 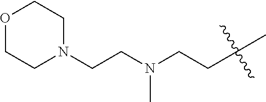 [SMILES: *CCN(C)CCN1CCOCC1] |
| —OCH₂CH₂OCH₂CH₂OH | —NH |
| —OCH₂CH₂OCH₂CH₂OMe | —NHMe |
| —OCH₂CH₂OCH₂CH₂OEt | —NMe₂ |
| —OCH₂CH₂OCH₂CH₂NH₂ | —NHEt |
| —OCH₂CH₂OCH₂CH₂NHMe | —NHPr |
| —OCH₂CH₂OCH₂CH₂NMe₂ | —NHiPr |
| 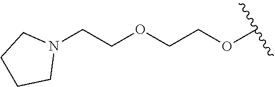 [SMILES: *OCCOCCN1CCCC1] | —NHcPr |
| 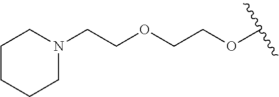 [SMILES: *OCCOCCN1CCCCC1] | —NHcBu |
| 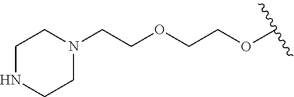 [SMILES: *OCCOCCN1CCNCC1] | —NHcPn |
| 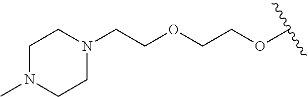 [SMILES: *OCCOCCN1CCN(C)CC1] | —NHcHex |
| 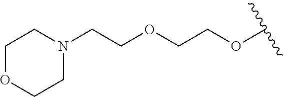 [SMILES: *OCCOCCN1CCOCC1] | —NHCH₂CH₂OH |
| —OCH₂CH₂OCH₂CH₂CH₂OH | —NHCH₂CH₂OMe |
| —OCH₂CH₂OCH₂CH₂CH₂OMe | —NHCH₂CH₂OEt |
| —OCH₂CH₂OCH₂CH₂CH₂OEt | —NHCH₂CH₂NH₂ |
| —OCH₂CH₂OCH₂CH₂CH₂NH₂ | —NHCH₂CH₂NHMe |
| —OCH₂CH₂OCH₂CH₂CH₂NHMe | —NHCH₂CH₂NMe₂ |
| —OCH₂CH₂OCH₂CH₂CH₂NMe₂ | 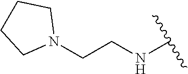 [SMILES: *NCCN1CCCC1] |
| 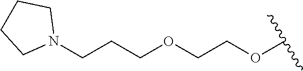 [SMILES: *OCCOCCCN1CCCC1] | 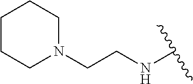 [SMILES: *NCCN1CCCCC1] |
| 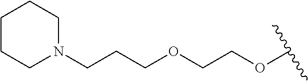 [SMILES: *OCCOCCCN1CCCCC1] | 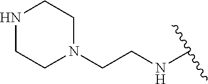 [SMILES: *NCCN1CCNCC1] |
| 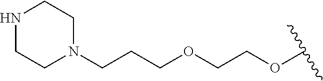 [SMILES: *OCCOCCCN1CCNCC1] | 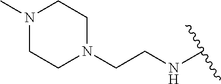 [SMILES: *NCCN1CCN(C)CC1] |
| 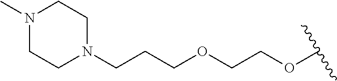 [SMILES: *OCCOCCCN1CCN(C)CC1] | 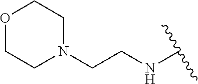 [SMILES: *NCCN1CCOCC1] |
| 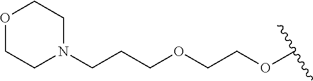 [SMILES: *OCCOCCCN1CCOCC1] | —NHCH₂CH₂CH₂OH |
| —OCH₂CH₂CH₂OCH₂CH₂OH | —NHCH₂CH₂CH₂OMe |
| —OCH₂CH₂CH₂OCH₂CH₂OMe | —NHCH₂CH₂CH₂OEt |
| —OCH₂CH₂CH₂OCH₂CH₂OEt | —NHCH₂CH₂CH₂NH₂ |
| —OCH₂CH₂CH₂OCH₂CH₂NH₂ | —NHCH₂CH₂CH₂NHMe |
| —OCH₂CH₂CH₂OCH₂CH₂NHMe | —NHCH₂CH₂CH₂NMe₂ |
| —OCH₂CH₂CH₂OCH₂CH₂NMe₂ | 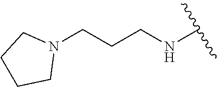 [SMILES: *NCCCN1CCCC1] |
| 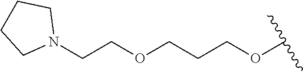 [SMILES: *OCCCOCCN1CCCC1] | 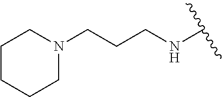 [SMILES: *NCCCN1CCCCC1] |
| 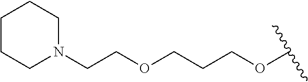 [SMILES: *OCCCOCCN1CCCCC1] | 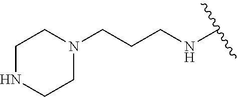 [SMILES: *NCCCN1CCNCC1] |
| 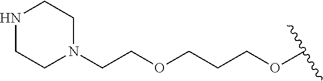 [SMILES: *OCCCOCCN1CCNCC1] | 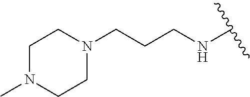 [SMILES: *NCCCN1CCN(C)CC1] |
| 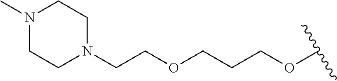 [SMILES: *OCCCOCCN1CCN(C)CC1] | 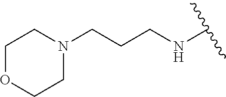 [SMILES: *NCCCN1CCOCC1] |
| 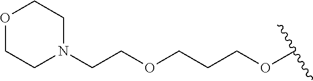 [SMILES: *OCCCOCCN1CCOCC1] | —NHCH₂CH₂OCH₂CH₂OH |
| —OCH₂CH₂CH₂OCH₂CH₂CH₂OH | —NHCH₂CH₂OCH₂CH₂OMe |
| —OCH₂CH₂CH₂OCH₂CH₂CH₂OMe | —NHCH₂CH₂OCH₂CH₂OEt |
| —OCH₂CH₂CH₂OCH₂CH₂CH₂OEt | —NHCH₂CH₂OCH₂CH₂NH₂ |
| —OCH₂CH₂CH₂OCH₂CH₂CH₂NH₂ | —NHCH₂CH₂OCH₂CH₂NHMe |
| —OCH₂CH₂CH₂OCH₂CH₂CH₂NHMe | —NHCH₂CH₂OCH₂CH₂NMe₂ |
| —OCH₂CH₂CH₂OCH₂CH₂CH₂NMe₂ | —N(CH₂CH₂OH)₂ |
| 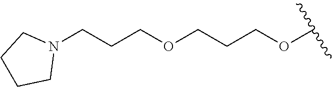 [SMILES: *OCCCOCCCN1CCCC1] | —N(CH₂CH₂OMe)₂ |
| 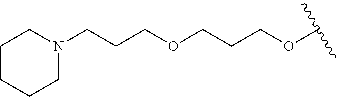 [SMILES: *OCCCOCCCN1CCCCC1] | —N(CH₂CH₂OEt)₂ |
| 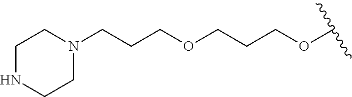 [SMILES: *OCCCOCCCN1CCNCC1] | —N(CH₂CH₂CH₂OH)₂ |
| 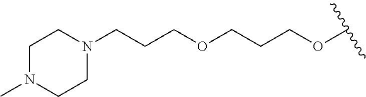 [SMILES: *OCCCOCCCN1CCN(C)CC1] | —N(CH₂CH₂CH₂OMe)₂ |
| 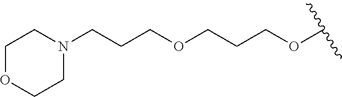 [SMILES: *OCCCOCCCN1CCOCC1] | —N(CH₂CH₂CH₂OEt)₂ |
| —OCH₂CH₂NHCH₂CH₂OH | —N(CH₂CH₂CH₂NH₂)₂ |
| —OCH₂CH₂NHCH₂CH₂OMe | —N(CH₂CH₂CH₂NHMe)₂ |
| —OCH₂CH₂NHCH₂CH₂OEt | —N(CH₂CH₂CH₂NMe₂)₂ |
| —OCH₂CH₂NHCH₂CH₂NH₂ | 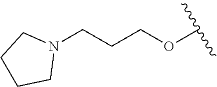 [SMILES: *OCCCN1CCCC1] |
| —OCH₂CH₂NHCH₂CH₂NHMe | 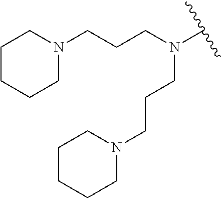 [SMILES: *N(CCCN1CCCCC1)CCCN1CCCCC1] |
| —OCH₂CH₂NHCH₂CH₂NMe₂ | 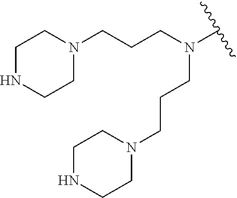 [SMILES: *N(CCCN1CCNCC1)CCCN1CCNCC1] |
| 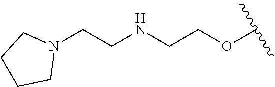 [SMILES: *OCCNCCN1CCCC1] | 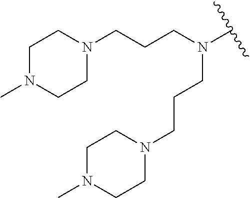 [SMILES: *N(CCCN1CCN(C)CC1)CCCN1CCN(C)CC1] |
| 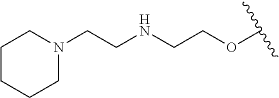 [SMILES: *OCCNCCN1CCCCC1] | 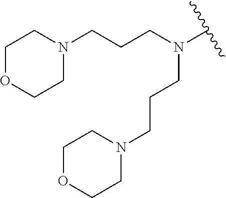 [SMILES: *N(CCCN1CCOCC1)CCCN1CCOCC1] |
| 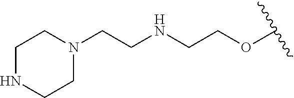 [SMILES: *OCCNCCN1CCNCC1] | —N(CH₂CH₂OCH₂CH₂OH)₂ |
| 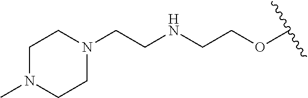 [SMILES: *OCCNCCN1CCN(C)CC1] | —N(CH₂CH₂OCH₂CH₂OMe)₂ |
| 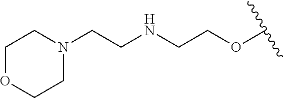 [SMILES: *OCCNCCN1CCOCC1] | —N(CH₂CH₂OCH₂CH₂OEt)₂ |
| —OCH₂CH₂NHCH₂CH₂CH₂OH | —N(CH₂CH₂OCH₂CH₂NH₂)₂ |
| —OCH₂CH₂NHCH₂CH₂CH₂OMe | —N(CH₂CH₂OCH₂CH₂NHMe)₂ |
| —OCH₂CH₂NHCH₂CH₂CH₂OEt | —N(CH₂CH₂OCH₂CH₂NMe₂)₂ |
| —OCH₂CH₂NHCH₂CH₂CH₂NH₂ | —NMeCH₂CH₂OH |
| —OCH₂CH₂NHCH₂CH₂CH₂NHMe | —NMeCH₂CH₂OMe |
| —OCH₂CH₂NHCH₂CH₂CH₂NMe₂ | —NMeCH₂CH₂OEt |
| 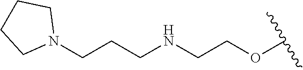 [SMILES: *OCCNCCCN1CCCC1] | —NMeCH₂CH₂NH₂ |
| 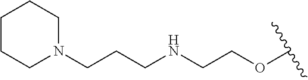 [SMILES: *OCCNCCCN1CCCCC1] | —NMeCH₂CH₂NHMe |
| 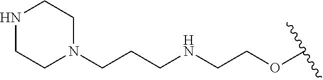 [SMILES: *OCCNCCCN1CCNCC1] | —NMeCH₂CH₂NMe₂ |
| 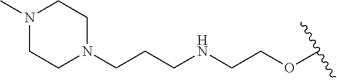 [SMILES: *OCCNCCCN1CCN(C)CC1] | 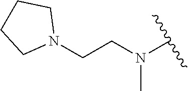 [SMILES: *N(C)CCN1CCCC1] |
| 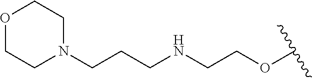 [SMILES: *OCCNCCCN1CCOCC1] | 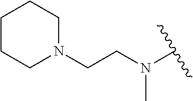 [SMILES: *N(C)CCN1CCCCC1] |
| —OCH₂CH₂CH₂NHCH₂CH₂OH | 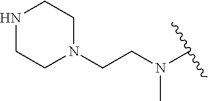 [SMILES: *N(C)CCN1CCNCC1] |
| —OCH₂CH₂CH₂NHCH₂CH₂OMe | 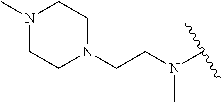 [SMILES: *N(C)CCN1CCN(C)CC1] |
| —OCH₂CH₂CH₂NHCH₂CH₂OEt | 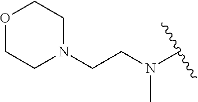 [SMILES: *N(C)CCN1CCOCC1] |
| —OCH₂CH₂CH₂NHCH₂CH₂NH₂ | —NMeCH₂CH₂CH₂OH |
| —OCH₂CH₂CH₂NHCH₂CH₂NHMe | —NMeCH₂CH₂CH₂OMe |
| —OCH₂CH₂CH₂NHCH₂CH₂NMe₂ | —NMeCH₂CH₂CH₂OEt |
| 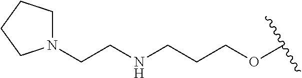 [SMILES: *OCCCNCCN1CCCC1] | —NMeCH₂CH₂CH₂NH₂ |
| 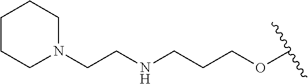 [SMILES: *OCCCNCCN1CCCCC1] | —NMeCH₂CH₂CH₂NHMe |
| 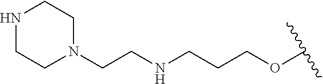 [SMILES: *OCCCNCCN1CCNCC1] | —NMeCH₂CH₂CH₂NMe₂ |
| 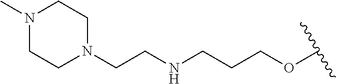 [SMILES: *OCCCNCCN1CCN(C)CC1] | 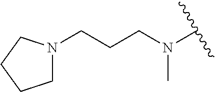 [SMILES: *N(C)CCCN1CCCC1] |
| 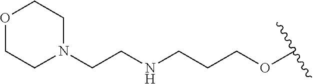 [SMILES: *OCCCNCCN1CCOCC1] | 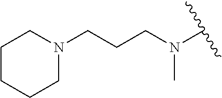 [SMILES: *N(C)CCCN1CCCCC1] |
| —OCH₂CH₂CH₂NHCH₂CH₂CH₂OH | 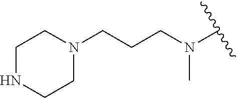 [SMILES: *N(C)CCCN1CCNCC1] |
| —OCH₂CH₂CH₂NHCH₂CH₂CH₂OMe | 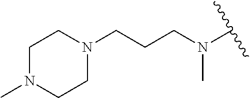 [SMILES: *N(C)CCCN1CCN(C)CC1] |
| —OCH₂CH₂CH₂NHCH₂CH₂CH₂OEt | 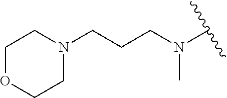 [SMILES: *N(C)CCCN1CCOCC1] |
| —OCH₂CH₂CH₂NHCH₂CH₂CH₂NH₂ | —NMeCH₂CH₂OCH₂CH₂OH |
| —OCH₂CH₂CH₂NHCH₂CH₂CH₂NHMe | —NMeCH₂CH₂OCH₂CH₂OMe |
| —OCH₂CH₂CH₂NHCH₂CH₂CH₂NMe₂ | —NMeCH₂CH₂OCH₂CH₂OEt |
| 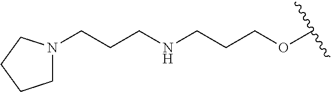 [SMILES: *OCCCNCCCN1CCCC1] | —NMeCH₂CH₂OCH₂CH₂NH₂ |
| 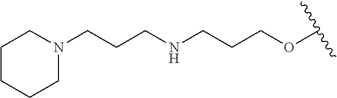 [SMILES: *OCCCNCCCN1CCCCC1] | —NMeCH₂CH₂OCH₂CH₂NHMe |
| 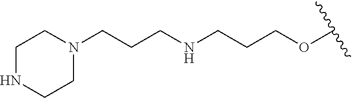 [SMILES: *OCCCNCCCN1CCNCC1] | —NMeCH₂CH₂OCH₂CH₂NMe₂ |
| 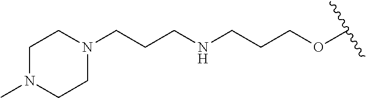 [SMILES: *OCCCNCCCN1CCN(C)CC1] | —O(C═O)OMe |
| 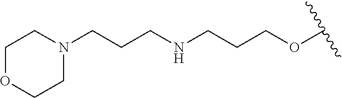 [SMILES: *OCCCNCCCN1CCOCC1] | —O(C═O)OEt |
| —OCH₂CH₂N(CH₂CH₂OH)₂ | —O(C═O)OnPr |
| —OCH₂CH₂N(CH₂CH₂OMe)₂ | —O(C═O)OiPr |
| —OCH₂CH₂N(CH₂CH₂OEt)₂ | —O(C═O)OcPr |
| —OCH₂CH₂N(CH₂CH₂CH₂OH)₂ | —O(C═O)OcBu |
| —OCH₂CH₂N(CH₂CH₂CH₂OMe)₂ | —O(C═O)OcPn |
| —OCH₂CH₂N(CH₂CH₂CH₂OEt)₂ | —O(C═O)OcHex |
| —OCH₂CH₂CH₂(HCH₂CH₂OH)₂ | —COOH |
| —OCH₂CH₂CH₂N(CH₂CH₂OMe)₂ | —CH₂(C═O)OH |
| —OCH₂CH₂CH₂N(CH₂CH₂OEt)₂ | —CH₂(C═O)OMe |
| —OCH₂CH₂CH₂N(CH₂CH₂CH₂OH)₂ | —CH₂(C═O)OEt |
| —OCH₂CH₂CH₂N(CH₂CH₂CH₂OMe)₂ | —CH₂(C═O)OnPr |
| —OCH₂CH₂CH₂N(CH₂CH₂CH₂OEt)₂ | —CH₂(C═O)OiPr |
| —OCH₂CH₂NMeCH₂CH₂OH | —CH₂(C═O)OcPr |
| —OCH₂CH₂NMeCH₂CH₂OMe | —CH₂(C═O)OcBu |
| —OCH₂CH₂NMeCH₂CH₂OEt | —CH₂(C═O)OcPn |
| —OCH₂CH₂NMeCH₂CH₂NH₂ | —CH₂(C═O)OcHex |
| —OCH₂CH₂NMeCH₂CH₂NHMe | —OCH₂(C═O)OH |
| —OCH₂CH₂NMeCH₂CH₂NMe₂ | —OCH₂(C═O)OMe |
| 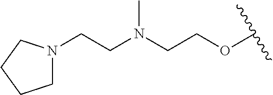 [SMILES: *OCCN(C)CCN1CCCC1] | —OCH₂(C═O)OEt |
| 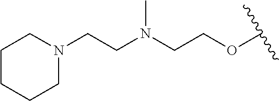 [SMILES: *OCCN(C)CCN1CCCCC1] | —OCH₂(C═O)OnPr |
| 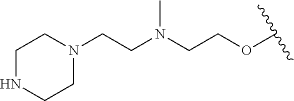 [SMILES: *OCCN(C)CCN1CCNCC1] | —OCH₂(C═O)OiPr |
| 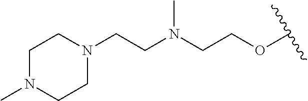 [SMILES: *OCCN(C)CCN1CCN(C)CC1] | —OCH₂(C═O)OcPr |
| 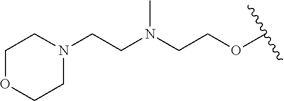 [SMILES: *OCCN(C)CCN1CCOCC1] | —OCH₂(C═O)OcBu |
| —OCH₂CH₂NMeCH₂CH₂CH₂OH | —OCH₂(C═O)OcPn |
| —OCH₂CH₂NMeCH₂CH₂CH₂OMe | —OCH₂(C═O)OcHex |
| —OCH₂CH₂NMeCH₂CH₂CH₂OEt | —OCH₂(C═O)OH |
| —OCH₂CH₂NMeCH₂CH₂CH₂NH₂ | —OCH₂(C═O)OMe |
| —OCH₂CH₂NMeCH₂CH₂CH₂NHMe | —OCH₂(C═O)OEt |
| —OCH₂CH₂NMeCH₂CH₂CH₂NMe₂ | —OCH₂(C═O)OnPr |
| 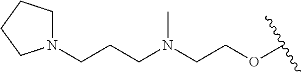 [SMILES: *OCCN(C)CCCN1CCCC1] | —OCH₂(C═O)OiPr |
| 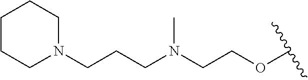 [SMILES: *OCCN(C)CCCN1CCCCC1] | —OCH₂(C═O)OcPr |
| 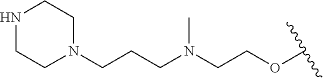 [SMILES: *OCCN(C)CCCN1CCNCC1] | —OCH₂(C═O)OcBu |
| 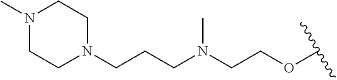 [SMILES: *OCCN(C)CCCN1CCN(C)CC1] | —OCH₂(C═O)OcPn |
| 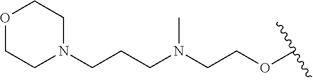 [SMILES: *OCCN(C)CCCN1CCOCC1] | —OCH₂(C═O)OcHex |
| —OCH₂CH₂CH₂NMeCH₂CH₂OH | —NHCH₂(C═O)OH |
| —OCH₂CH₂CH₂NMeCH₂CH₂OMe | —NHCH₂(C═O)OMe |
| —OCH₂CH₂CH₂NMeCH₂CH₂OEt | —NHCH₂(C═O)OEt |
| —OCH₂CH₂CH₂NMeCH₂CH₂NH₂ | —NHCH₂(C═O)OnPr |
| —OCH₂CH₂CH₂NMeCH₂CH₂NHMe | —NHCH₂(C═O)OiPr |
| —OCH₂CH₂CH₂NMeCH₂CH₂NMe₂ | —NHCH₂(C═O)OcPr |
| 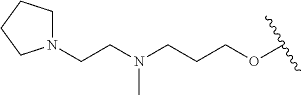 [SMILES: *OCCCN(C)CCN1CCCC1] | —NHCH₂(C═O)OcBu |
| 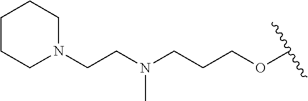 [SMILES: *OCCCN(C)CCN1CCCCC1] | —NHCH₂(C═O)OcPn |
| 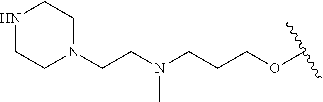 [SMILES: *OCCCN(C)CCN1CCNCC1] | —NHCH₂(C═O)OcHex |
| 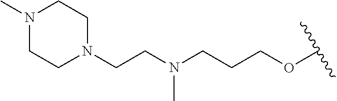 [SMILES: *OCCCN(C)CCN1CCN(C)CC1] | —NMeCH₂(C═O)OH |
| 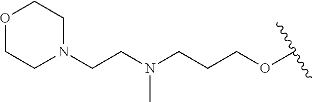 [SMILES: *OCCCN(C)CCN1CCOCC1] | —NH(CH₂(C═O)OH)₂ |
| —OCH₂CH₂CH₂NMeCH₂CH₂CH₂OH | —CH₂CH₂(C═O)OH |
| —OCH₂CH₂CH₂NMeCH₂CH₂CH₂OMe | —CH₂OCH₂(C═O)OH |
| —OCH₂CH₂CH₂NMeCH₂CH₂CH₂OEt | —OCH₂CH₂(C═O)OH |
| —OCH₂CH₂CH₂NMeCH₂CH₂CH₂NH₂ | —CH₂CH₂CH₂(C═O)OH |
| —OCH₂CH₂CH₂NMeCH₂CH₂CH₂NHMe | —CH₂CH₂OCH₂(C═O)OH |
| —OCH₂CH₂CH₂NMeCH₂CH₂CH₂NMe₂ | —CH₂OCH₂CH₂(C═O)OH |
| 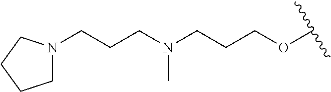 [SMILES: *OCCCN(C)CCCN1CCCC1] | —OCH₂CH₂CH₂(C═O)OH |
| 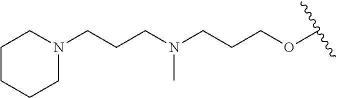 [SMILES: *OCCCN(C)CCCN1CCCCC1] | —OCH₂CH₂OCH₂(C═O)OH |
| 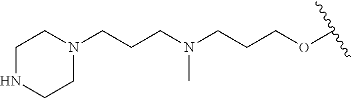 [SMILES: *OCCCN(C)CCCN1CCNCC1] | —CH₂CH₂CH₂CH₂(C═O)OH |
| 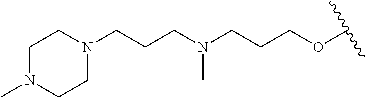 [SMILES: *OCCCN(C)CCCN1CCN(C)CC1] | —CH₂CH₂CH₂OCH₂(C═O)OH |
| 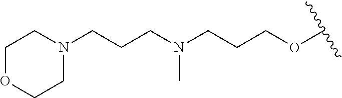 [SMILES: *OCCCN(C)CCCN1CCOCC1] | —CH₂CH₂OCH₂CH₂(C═O)OH |
| —CH₂OH | —CH₂OCH₂CH₂CH₂(C═O)OH |
| —CH₂OMe | —OCH₂CH₂CH₂CH₂(C═O)OH |
| —CH₂OEt | —CH₂OCH₂CH₂OCH₂(C═O)OH |
| —CH₂NH₂ | —OCH₂CH₂CH₂OCH₂(C═O)OH |
| —CH₂NHMe | —OCH₂CH₂OCH₂CH₂(C═O)OH |
| —CH₂NMe₂ | —CH₂OCH₂CH₂CH₂(C═O)OH |
| 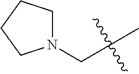 [SMILES: *CN1CCCC1] | —CH₂CH₂CH₂CH₂CH₂(C═O)OH |
| 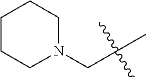 [SMILES: *CN1CCCCC1] | —CH₂CH₂CH₂CH₂OCH₂(C═O)OH |
| 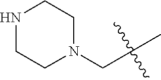 [SMILES: *CN1CCNCC1] | —CH₂CH₂CH₂OCH₂CH₂(C═O)OH |
| 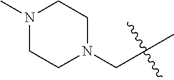 [SMILES: *CN1CCN(C)CC1] | —CH₂CH₂OCH₂CH₂CH₂(C═O)OH |
| 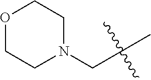 [SMILES: *CN1CCOCC1] | —CH₂OCH₂CH₂CH₂CH₂(C═O)OH |
| —CH₂CH₂OH | —OCH₂CH₂CH₂CH₂CH₂(C═O)OH |
| —CH₂CH₂OMe | —CH₂CH₂OCH₂CH₂OCH₂(C═O)OH |
| —CH₂CH₂OEt | —CH₂OCH₂CH₂CH₂OCH₂(C═O)OH |
| —CH₂CH₂NH₂ | —OCH₂CH₂CH₂CH₂OCH₂(C═O)OH |
| —CH₂CH₂NHMe | —CH₂OCH₂CH₂OCH₂CH₂(C═O)OH |
| —CH₂CH₂NMe₂ | —OCH₂CH₂CH₂OCH₂CH₂(C═O)OH |
| 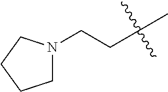 [SMILES: *CCN1CCCC1] | —OCH₂CH₂OCH₂CH₂CH₂(C═O)OH |
| 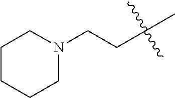 [SMILES: *CCN1CCCCC1] | —OCH₂CH₂OCH₂CH₂OCH₂(C═O)OH |
| 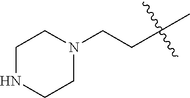 [SMILES: *CCN1CCNCC1] | —CH₂CH₂CH₂CH₂CH₂CH₂(C═O)OH |
| 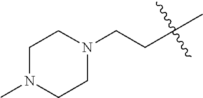 [SMILES: *CCN1CCN(C)CC1] | —CH₂CH₂CH₂CH₂CH₂OCH₂(C═O)OH |
| 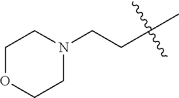 [SMILES: *CCN1CCOCC1] | —CH₂CH₂CH₂OCH₂CH₂OCH₂(C═O)OH |
| —CH₂CH₂CH₂OH | —CH₂OCH₂CH₂OCH₂CH₂OCH₂C(O)OH |
| —CH₂CH₂CH₂OMe | —OCH₂CH₂CH₂OCH₂CH₂OCH₂C(O)OH |
| —CH₂CH₂CH₂OEt | —CH₂CH₂CH₂CH₂OCH₂CH₂(C═O)OH |
| —CH₂CH₂CH₂NH₂ | —CH₂CH₂OCH₂CH₂OCH₂CH₂(C═O)OH |
| —CH₂CH₂CH₂NHMe | —CH₂OCH₂CH₂CH₂OCH₂CH₂(C═O)OH |
| —CH₂CH₂CH₂NMe₂ | —OCH₂CH₂CH₂CH₂OCH₂CH₂(C═O)OH |
| 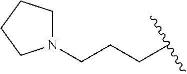 [SMILES: *CCCN1CCCC1] | —OCH₂CH₂OCH₂CH₂OCH₂CH₂(CO)OH |
| 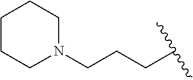 [SMILES: *CCCN1CCCCC1] | —CH₂CH₂CH₂OCH₂CH₂CH₂(C═O)OH |
| 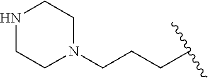 [SMILES: *CCCN1CCNCC1] | —CH₂OCH₂CH₂OCH₂CH₂CH₂(C═O)OH |
| 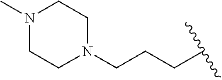 [SMILES: *CCCN1CCN(C)CC1] | —OCH₂CH₂CH₂OCH₂CH₂CH₂(C═O)OH |
| 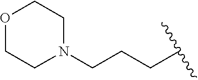 [SMILES: *CCCN1CCOCC1] | —CH₂CH₂OCH₂CH₂CH₂CH₂(C═O)OH |
| —CH₂CH₂CH₂CH₂OH | —OCH₂CH₂OCH₂CH₂CH₂CH₂(C═O)OH |
| —CH₂CH₂CH₂CH₂OMe | —CH₂OCH₂CH₂CH₂CH₂CH₂(C═O)OH |
| —CH₂CH₂CH₂CH₂OEt | —OCH₂CH₂CH₂CH₂CH₂CH₂(C═O)OH |
| —CH₂CH₂CH₂CH₂NH₂ | —NHCH₂CH₂(C═O)OH |
| —CH₂CH₂CH₂CH₂NHMe | —NMeCH₂CH₂(C═O)OH |
| —CH₂CH₂CH₂CH₂NMe₂ | —N(CH₂CH₂(C═O)OH)₂ |
| 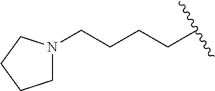 [SMILES: *CCCCN1CCCC1] | —NHCH₂CH₂OCH₂(C═O)OH |
| 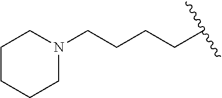 [SMILES: *CCCCN1CCCCC1] | —NMeCH₂CH₂OCH₂(C═O)OH |
| 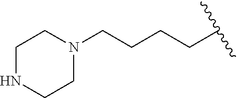 [SMILES: *CCCCN1CCNCC1] | —N(CH₂CH₂OCH₂(C═O)OH)₂ |
| 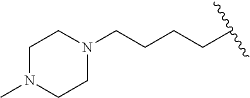 [SMILES: *CCCCN1CCN(C)CC1] | —NHCH₂CH₂CH₂CH₂(C═O)OH |
| 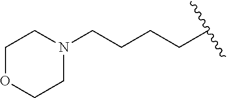 [SMILES: *CCCCN1CCOCC1] | —NMeCH₂CH₂CH₂CH₂(C═O)OH |
| —CH₂OCH₂CH₂OH | —N(CH₂CH₂CH₂CH₂(C═O)OH)₂ |
| —CH₂OCH₂CH₂OMe | —NHCH₂CH₂CH₂OCH₂(C═O)OH |
| —CH₂OCH₂CH₂OEt | —NMeCH₂CH₂CH₂OCH₂(C═O)OH |
| —CH₂OCH₂CH₂NH₂ | —N(CH₂CH₂CH₂OCH₂(C═O)OH)₂ |
| —CH₂OCH₂CH₂NHMe | —NHCH₂CH₂OCH₂CH₂(C═O)OH |
| —CH₂OCH₂CH₂NMe₂ | —NMeCH₂CH₂OCH₂CH₂(C═O)OH |
| 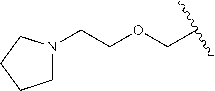 [SMILES: *COCCN1CCCC1] | —N(CH₂CH₂OCH₂CH₂(C═O)OH)₂ |
| 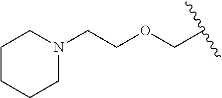 [SMILES: *COCCN1CCCCC1] | —NHCH₂CH₂CH₂CH₂CH₂(C═O)OH |
| 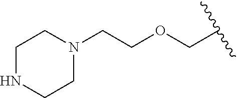 [SMILES: *COCCN1CCNCC1] | —NMeCH₂CH₂CH₂CH₂CH₂(C═O)OH |
| 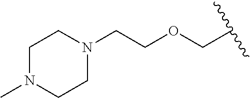 [SMILES: *COCCN1CCN(C)CC1] | —N(CH₂CH₂CH₂CH₂CH₂(C═O)OH)₂ |
| 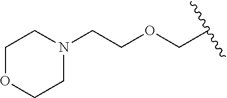 [SMILES: *COCCN1CCOCC1] | —NHCH₂CH₂CH₂CH₂OCH₂(C═O)OH |
| —CH₂NHCH₂CH₂OH | —NMeCH₂CH₂CH₂CH₂OCH₂(C═O)OH |
| —CH₂NHCH₂CH₂OMe | —N(CH₂CH₂CH₂CH₂OCH₂(C═O)OH)₂ |
| —CH₂NHCH₂CH₂OEt | —NHCH₂CH₂CH₂OCH₂CH₂(C═O)OH |
| —CH₂NHCH₂CH₂NH₂ | —NMeCH₂CH₂CH₂OCH₂CH₂(C═O)OH |
| —CH₂NHCH₂CH₂NHMe | —N(CH₂CH₂CH₂OCH₂CH₂(C═O)OH)₂ |
| —CH₂NHCH₂CH₂NMe₂ | —NHCH₂CH₂OCH₂CH₂CH₂(C═O)OH |
| 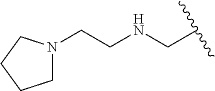 [SMILES: *CNCCN1CCCC1] | —NMeCH₂CH₂OCH₂CH₂CH₂(C═O)OH |
| 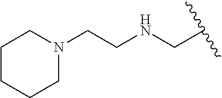 [SMILES: *CNCCN1CCCCC1] | —N(CH₂CH₂OCH₂CH₂CH₂(C═O)OH)₂ |
| 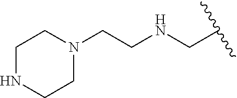 [SMILES: *CNCCN1CCNCC1] | —NHCH₂CH₂OCH₂CH₂OCH₂(C═O)OH |
| 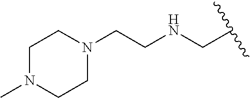 [SMILES: *CNCCN1CCN(C)CC1] | —NMeCH₂CH₂OCH₂CH₂OCH₂(C═O)OH |
| 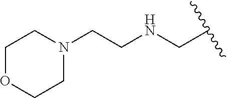 [SMILES: *CNCCN1CCOCC1] | —N(CH₂CH₂OCH₂CH₂OCH₂(C═O)OH)₂ |
| —CH₂N(CH₂CH₂OH)₂ | —NHCH₂CH₂CH₂OCH₂CH₂OCH₂CO₂H |
| —CH₂N(CH₂CH₂OMe)₂ | —NMeCH₂CH₂CH₂OCH₂CH₂OCH₂CO₂H |
| —CH₂N(CH₂CH₂OEt)₂ | —N(CH₂CH₂CH₂OCH₂CH₂OCH₂CO₂H)₂ |
| —CH₂NMeCH₂CH₂OH | —NHCH₂CH₂CH₂CH₂OCH₂CH₂CO₂H |
| —CH₂NMeCH₂CH₂OMe | —NMeCH₂CH₂CH₂CH₂OCH₂CH₂CO₂H |
| —CH₂NMeCH₂CH₂OEt | —N(CH₂CH₂CH₂CH₂OCH₂CH₂CO₂H)₂ |
| —CH₂NMeCH₂CH₂NH₂ | —NHCH₂CH₂OCH₂CH₂OCH₂CH₂CO₂H |
| —CH₂NMeCH₂CH₂NHMe | —NMeCH₂CH₂OCH₂CH₂OCH₂CH₂CO₂H |
| —CH₂NMeCH₂CH₂NMe₂ | —N(CH₂CH₂OCH₂CH₂OCH₂CH₂CO₂H)₂ |
| 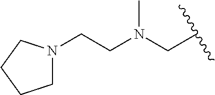 [SMILES: *CN(C)CCN1CCCC1] | —NHCH₂CH₂CH₂OCH₂CH₂CH₂CO₂H |
| 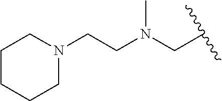 [SMILES: *CN(C)CCN1CCCCC1] | —NMeCH₂CH₂CH₂OCH₂CH₂CH₂CO₂H |
| 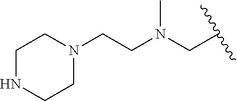 [SMILES: *CN(C)CCN1CCNCC1] | —N(CH₂CH₂CH₂OCH₂CH₂CH₂CO₂H)₂ |
| 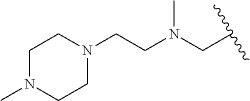 [SMILES: *CN(C)CCN1CCN(C)CC1] | —NHCH₂CH₂OCH₂CH₂CH₂CH₂CO₂H |
| 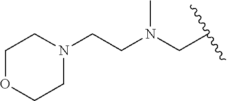 [SMILES: *CN(C)CCN1CCOCC1] | -NMeCH₂CH₂OCH₂CH₂CH₂CH₂CO₂H |
| —CH₂CH₂OCH₂CH₂OH | —N(CH₂CH₂OCH₂CH₂CH₂CH₂CO₂H)₂ |
| —CH₂CH₂OCH₂CH₂OMe | —NHCH₂CH₂CH₂CH₂CH₂CH₂CO₂H |
| —CH₂CH₂OCH₂CH₂OEt | —NMeCH₂CH₂CH₂CH₂CH₂CH₂CO₂H |
| —CH₂CH₂OCH₂CH₂NH₂ | —N(CH₂CH₂CH₂CH₂CH₂CH₂CO₂H)₂ |
| —CH₂CH₂OCH₂CH₂NHMe | |

Embodiment 14 provides the compound of any one of embodiments 1, having the structure:

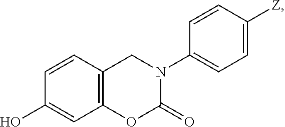

wherein Z is selected from the group consisting of

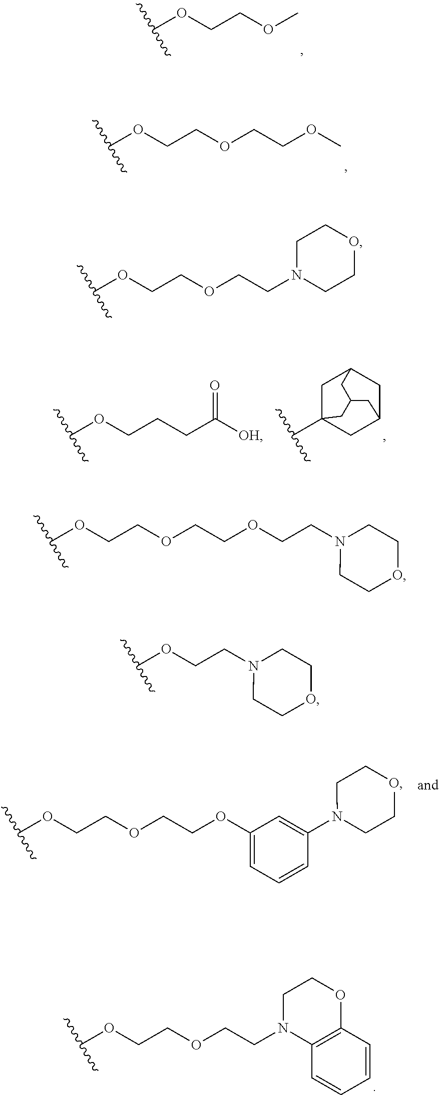

Embodiment 15 provides the compound of any one of embodiments 1-6 and 8-11, having the structure:

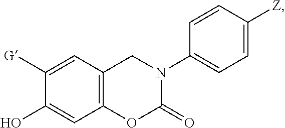

wherein Z is selected from the group consisting of

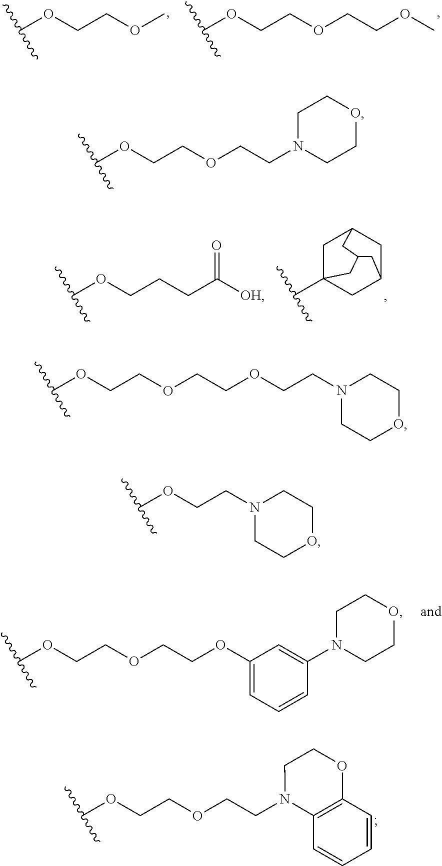

and
wherein G′ is halogen.

Embodiment 16 provides the compound of any one of embodiments 1-6, 8-11, and 15, wherein G′ is F.

Embodiment 17 provides the compound of any one of embodiments 1-6, 8-11, and 15-16, which is selected from the group consisting of:

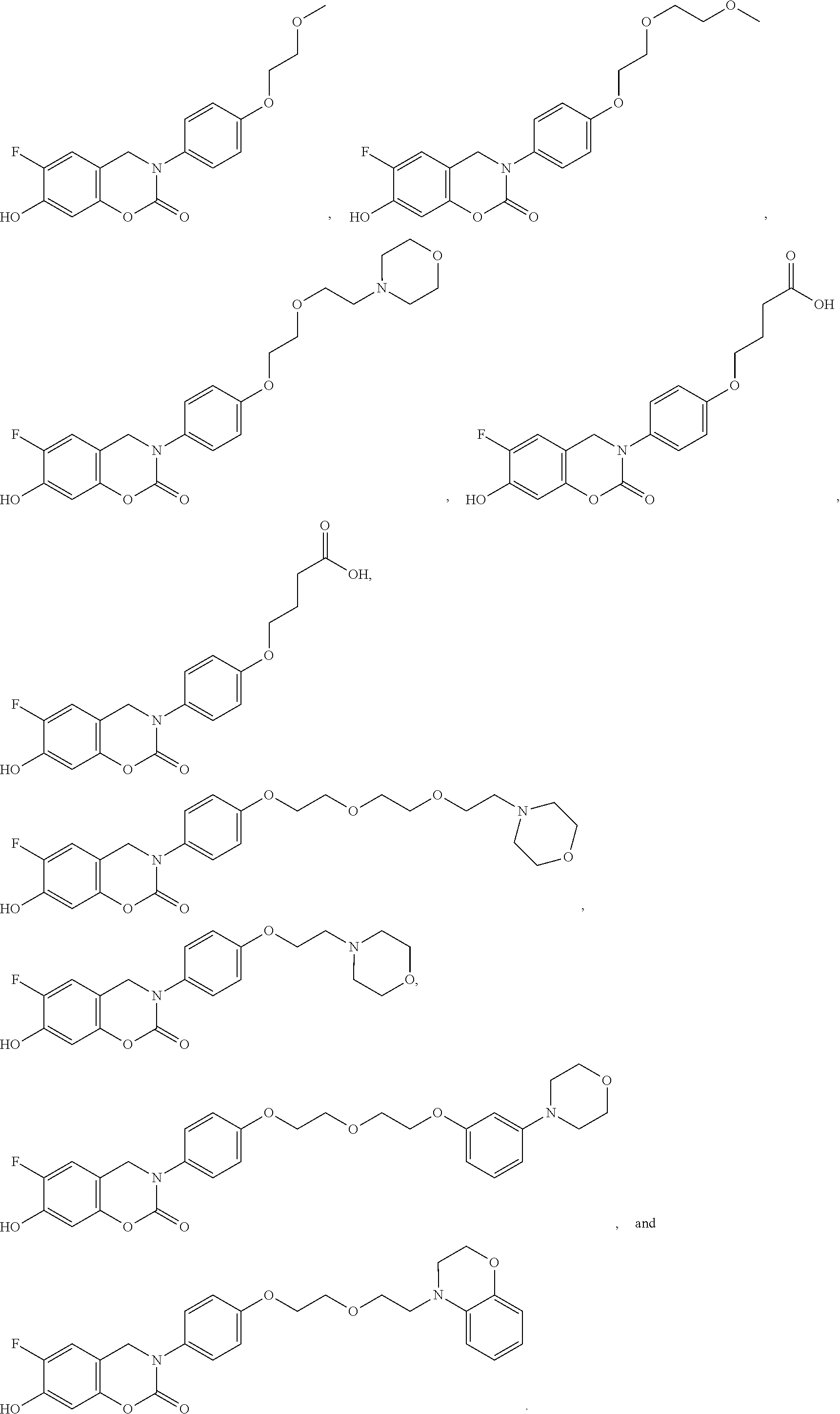

Embodiment 18 provides a pharmaceutical composition comprising the compound of any one of embodiments 1-17, or a salt, enantiomer, tautomer, or solvate thereof, and a pharmaceutically acceptable carrier.

Embodiment 19 provides a method of inhibiting macrophage migration inhibitory factor (MIF) activity in a subject, the method comprising:

administering to the subject an effective amount of the compound of any one of embodiments 1-18, or a salt, enantiomer, tautomer, or solvate thereof.

Embodiment 20 provides a method of treating a disease or condition in which inhibition of macrophage migration inhibitory factor (MIF) activity in a subject is therapeutically beneficial, the method comprising administering to the subject an effective amount of a composition comprising the compound of any one of embodiments 1-18, or a salt, enantiomer, tautomer, or solvate thereof,

wherein the disease or condition is selected from the group consisting of an inflammatory disease, an autoimmune disease, and cancer.

Embodiment 21 provides the method of any embodiment 20, wherein the inflammatory disease or condition is selected from the group consisting of proliferative vascular disease, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Berger's disease, Retier's syndrome, and Hodgkins disease.

Embodiment 22 provides the method of any one of embodiments 20-21, wherein the autoimmune disease is selected from the group consisting of multiple sclerosis, systemic lupus erythematosus, rheumatoid arthritis, graft versus host disease, autoimmune pulmonary inflammation, autoimmune encephalomyelitis, Guillain-Barre syndrome, autoimmune thyroiditis, insulin dependent diabetes mellitus, Crohn's disease, scleroderma, psoriasis, Sjögren's syndrome, and autoimmune inflammatory eye disease.

Embodiment 23 provides the method of any one of embodiments 20-22, wherein the cancer is a solid tumor or a hematological cancer.

Embodiment 24 provides the method of any one of embodiments 20-23, wherein the cancer is selected from the group consisting of prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head or neck, thyroid cancer, glioblastoma, sarcoma, bladder cancer, lymphoma, leukemia, diffuse large B-cell lymphoma (DLBCL), mantle cell lymphoma, non-Hodgkin lymphoma, Hodgkin lymphoma, and multiple myeloma.

Embodiment 25 provides the method of any one of embodiments 20-24, wherein the composition is formulated for an administration route selected from the group consisting of oral, transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 26 provides the method of any one of embodiments 20-25, wherein the composition is formulated for oral administration.

Embodiment 27 provides the method of any one of embodiments 20-26, wherein the composition is in the form of a tablet, capsule, caplet, gel cap, troche, dispersion, suspension, solution, or syrup.

1. A compound of formula (I), or a salt, enantiomer, tautomer, or solvate thereof: 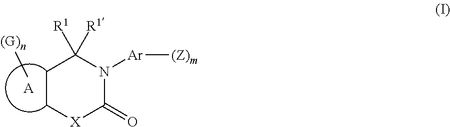
wherein:
R¹ and R^{1′} are each independently H, C₁₋₃ alkyl, or halogen;
X is CH₂, O, or S;
A is a fused 5-membered heteroaryl ring or a fused 6-membered aryl or heteroaryl ring;
each G is independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, and combinations thereof;
Ar is a 5-membered heteroaryl or a 6-membered aryl or heteroaryl ring;
each Z is a substituent on Ar independently selected from the group consisting of halogen, C₁₋₁₀ alkyl, C₂₋₁₀ alkenyl, C₂₋₁₀ alkynyl, C₃₋₇ cycloalkyl, C₆₋₁₀ aryl, 5-10 membered heteroaryl, 4-10 membered heterocyclyl, C₃₋₁₀ cycloalkyl-C₁₋₁₀ alkyl, C₃₋₁₀ cycloalkyl-C₁₋₁₀ heteroalkyl, C₆₋₁₀ aryl-C₁₋₁₀ alkyl, C₆₋₁₀ aryl-C₁₋₁₀ heteroalkyl, 5-10 membered heteroaryl-C₁₋₁₀ alkyl, 5-10 membered heteroaryl-C₁₋₁₀ heteroalkyl, 4-10 membered heterocyclyl-C₁₋₁₀ alkyl, 4-10 membered heterocyclyl-C₁₋₁₀ heteroalkyl, CN, NO₂, OR, SR, C(O)R, C(O)NRR′, C(O)OR, OC(O)R, OC(O)NRR′, NRR′, NRC(O)R, NRC(O)NRR′, NRC(O)OR, C(═NR)NRR′, NRC(═NR)NRR′, S(O)R, S(O)₂R, NRS(O)₂R, S(O)₂NRR′, —(OCH₂CH₂)ₚ-4-10 membered heterocyclyl, —O(CH₂CH₂O)ₚ-4-10 membered heterocyclyl, —(OCH₂CH₂)ₚ-4-10 membered heteroaryl, —O(CH₂CH₂O)ₚ-4-10 membered heteroaryl, —O(CH₂CH₂O)ₚ—C₁₋₄ alkyl, and combinations thereof;
each R and R′ is independently H or C₁₋₁₀ hydrocarbyl;
n is an integer from 1 to 4;
m is an integer from 1 to 5; and
p is an integer from 1 to 5;
with the proviso that if
Ar is phenyl;
A is a fused 6-membered aryl ring having the structure 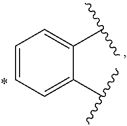 [SMILES: *c1ccccc1*]
substituted at the position marked with * by hydrogen, hydroxy, Ci-s alkoxy, Ci-s haloalkyl, or C₁₋₅ haloalkoxy; and
m is 1;
then Z is not OH, C₁₋₅ alkyl, C₁₋₅ alkoxy, C₁₋₅ haloalkyl, C₁₋₅ haloalkoxy, OCF₃, phenyl, or halogen.

2. The compound of claim 1, wherein at least one of the following applies:
i) the compound has the structure 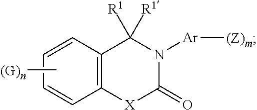
ii) X is O;
iii) R¹ and R^{1′} are H;
iv) the compound has the structure 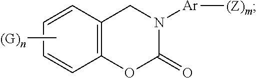
v) the compound has the structure 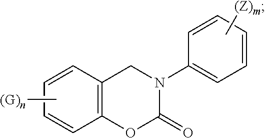
vi) the compound has the structure 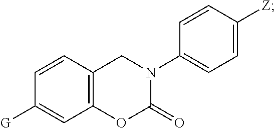
vii) the compound has the structure

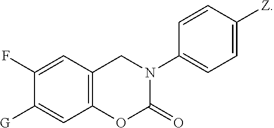

3-8. (canceled)

9. The compound of claim 2, wherein in vii) G is OH.

10. The compound of claim 1, wherein Z is selected from the group consisting of —(OCH₂CH₂)ₚ-4-10 membered non-aromatic heterocyclyl, —O(CH₂CH₂O)ₚ-4-10 membered non-aromatic heterocyclyl, —(OCH₂CH₂)ₚ-4-10 membered heteroaryl, —O(CH₂CH₂O)ₚ-4-10 membered heteroaryl, and —O(CH₂CH₂O)ₚ—C₁₋₄ alkyl.

11. The compound of claim 1, wherein Z is selected from the group consisting of

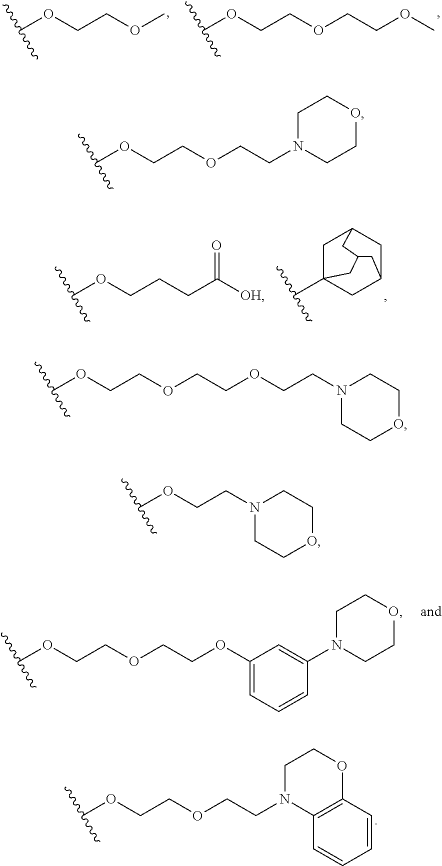

12. The compound of claim 1, which is selected from the group consisting of:

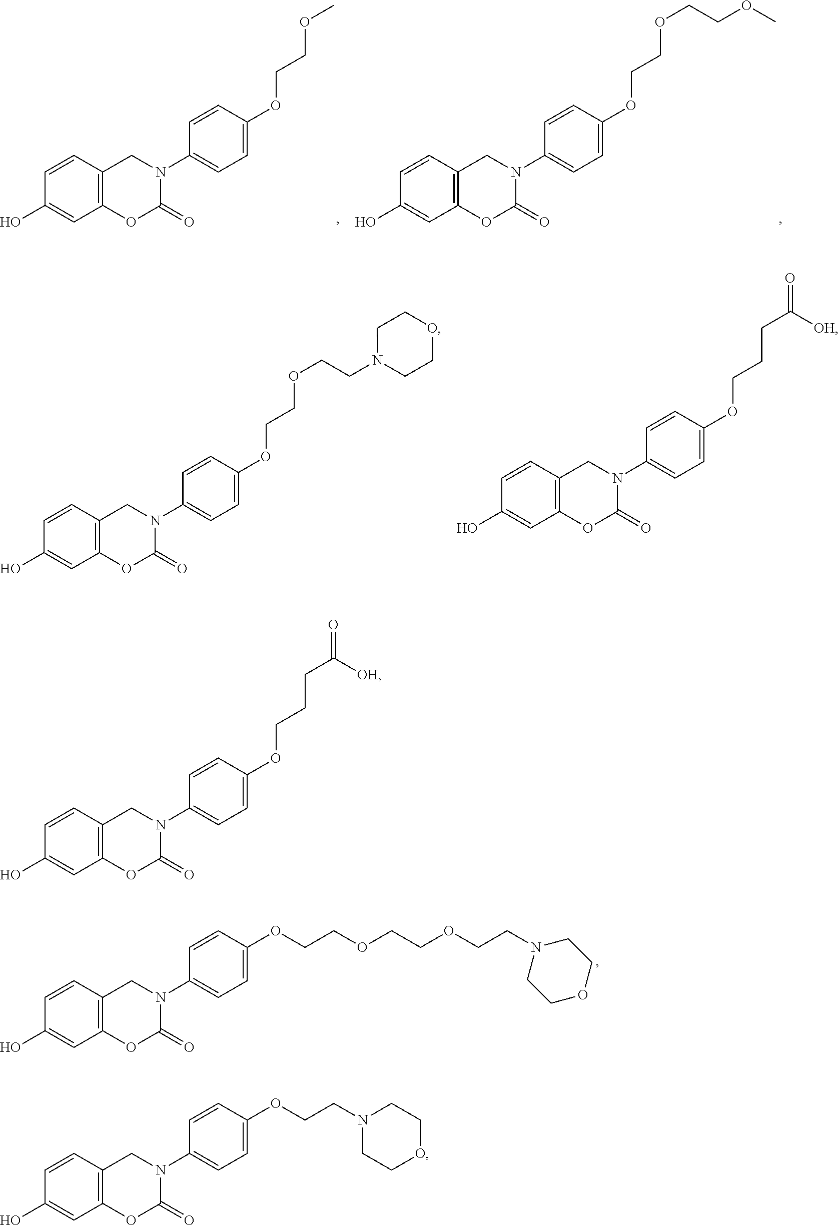

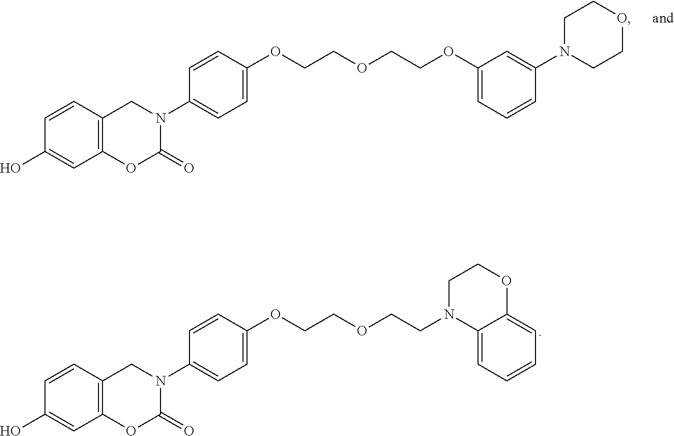 [SMILES: O=C1Oc2cc(O)ccc2CN1c1ccc(OCCOCCOc2cccc(N3CCOCC3)c2)cc1 ; O=C1Oc2cc(O)ccc2CN1c1ccc(OCCOCCN2CCOc3ccccc32)cc1]

13. The compound of claim 1, wherein Z is selected from the group consisting of

| | |
|---|---|
| | —CH₂CH₂OCH₂CH₂NMe₂ |
| —OH | 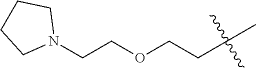 [SMILES: *CCOCCN1CCCC1] |
| —OEt | 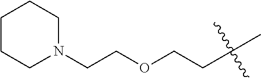 [SMILES: *CCOCCN1CCCCC1] |
| —OPr | 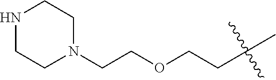 [SMILES: *CCOCCN1CCNCC1] |
| —OiPr | 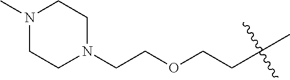 [SMILES: *CCOCCN1CCN(C)CC1] |
| —OcPr | 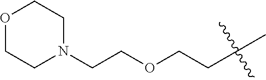 [SMILES: *CCOCCN1CCOCC1] |
| —OcBu | —CH₂CH₂NHCH₂CH₂OH |
| —OcPn | —CH₂CH₂NHCH₂CH₂OMe |
| —OcHex | —CH₂CH₂NHCH₂CH₂OEt |
| —OCH₂CH₂OH | —CH₂CH₂NHCH₂CH₂NH₂ |
| —OCH₂CH₂OMe | —CH₂CH₂NHCH₂CH₂NHMe |
| —OCH₂CH₂OEt | —CH₂CH₂NHCH₂CH₂NMe₂ |
| —OCH₂CH₂NH₂ | 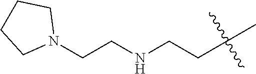 [SMILES: *CCNCCN1CCCC1] |
| —OCH₂CH₂NHMe | 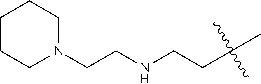 [SMILES: *CCNCCN1CCCCC1] |
| —OCH₂CH₂NMe₂ | 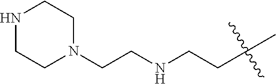 [SMILES: *CCNCCN1CCNCC1] |
| 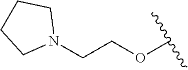 [SMILES: *OCCN1CCCC1] | 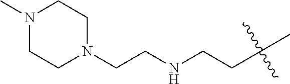 [SMILES: *CCNCCN1CCN(C)CC1] |
| 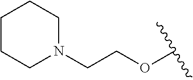 [SMILES: *OCCN1CCCCC1] | 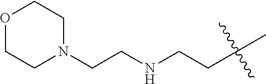 [SMILES: *CCNCCN1CCOCC1] |
| 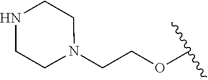 [SMILES: *OCCN1CCNCC1] | —CH₂CH₂N(CH₂CH₂OH)₂ |
| 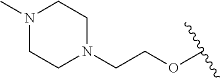 [SMILES: *OCCN1CCN(C)CC1] | —CH₂CH₂N(CH₂CH₂OMe)₂ |
| 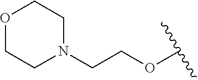 [SMILES: *OCCN1CCOCC1] | —CH₂CH₂N(CH₂CH₂OEt)₂ |
| —OCH₂CH₂CH₂OH | —CH₂CH₂NMeCH₂CH₂OH |
| —OCH₂CH₂CH₂OMe | —CH₂CH₂NMeCH₂CH₂OMe |
| —OCH₂CH₂CH₂OEt | —CH₂CH₂NMeCH₂CH₂OEt |
| —OCH₂CH₂CH₂NH₂ | —CH₂CH₂NMeCH₂CH₂NH₂ |
| —OCH₂CH₂CH₂NHMe | —CH₂CH₂NMeCH₂CH₂NHMe |
| —OCH₂CH₂CH₂NMe₂ | —CH₂CH₂NMeCH₂CH₂NMe₂ |
| 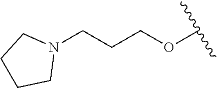 [SMILES: *OCCCN1CCCC1] | 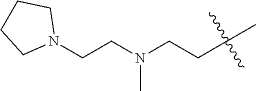 [SMILES: *CCN(C)CCN1CCCC1] |
| 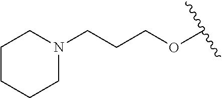 [SMILES: *OCCCN1CCCCC1] | 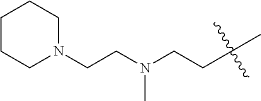 [SMILES: *CCN(C)CCN1CCCCC1] |
| 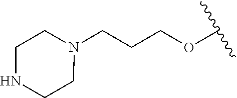 [SMILES: *OCCCN1CCNCC1] | 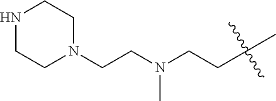 [SMILES: *CCN(C)CCN1CCNCC1] |
| 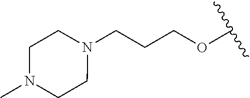 [SMILES: *OCCCN1CCN(C)CC1] | 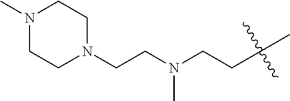 [SMILES: *CCN(C)CCN1CCN(C)CC1] |
| 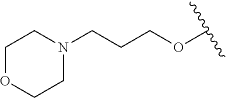 [SMILES: *OCCCN1CCOCC1] | 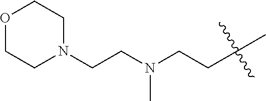 [SMILES: *CCN(C)CCN1CCOCC1] |
| —OCH₂CH₂OCH₂CH₂OH | —NH |
| —OCH₂CH₂OCH₂CH₂OMe | —NHMe |
| —OCH₂CH₂OCH₂CH₂OEt | —NMe₂ |
| —OCH₂CH₂OCH₂CH₂NH₂ | —NHEt |
| —OCH₂CH₂OCH₂CH₂NHMe | —NHPr |
| —OCH₂CH₂OCH₂CH₂NMe₂ | —NHiPr |
| 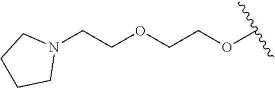 [SMILES: *OCCOCCN1CCCC1] | —NHcPr |
| 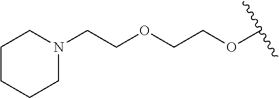 [SMILES: *OCCOCCN1CCCCC1] | —NHcBu |
| 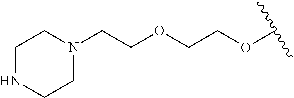 [SMILES: *OCCOCCN1CCNCC1] | —NHcPn |
| 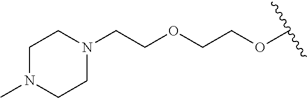 [SMILES: *OCCOCCN1CCN(C)CC1] | —NHcHex |
| 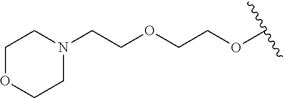 [SMILES: *OCCOCCN1CCOCC1] | —NHCH₂CH₂OH |
| —OCH₂CH₂OCH₂CH₂CH₂OH | —NHCH₂CH₂OMe |
| —OCH₂CH₂OCH₂CH₂CH₂OMe | —NHCH₂CH₂OEt |
| —OCH₂CH₂OCH₂CH₂CH₂OEt | —NHCH₂CH₂NH₂ |
| —OCH₂CH₂OCH₂CH₂CH₂NH₂ | —NHCH₂CH₂NHMe |
| —OCH₂CH₂OCH₂CH₂CH₂NHMe | —NHCH₂CH₂NMe₂ |
| —OCH₂CH₂OCH₂CH₂CH₂NMe₂ | 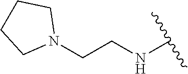 [SMILES: *NCCN1CCCC1] |
| 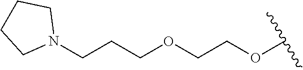 [SMILES: *OCCOCCCN1CCCC1] | 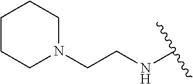 [SMILES: *NCCN1CCCCC1] |
| 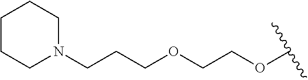 [SMILES: *OCCOCCCN1CCCCC1] | 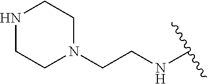 [SMILES: *NCCN1CCNCC1] |
| 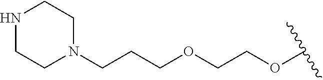 [SMILES: *OCCOCCCN1CCNCC1] | 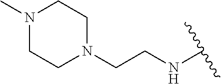 [SMILES: *NCCN1CCN(C)CC1] |
| 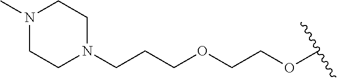 [SMILES: *OCCOCCCN1CCN(C)CC1] | 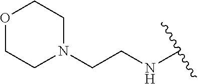 [SMILES: *NCCN1CCOCC1] |
| 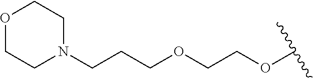 [SMILES: *OCCOCCCN1CCOCC1] | —NHCH₂CH₂CH₂OH |
| —OCH₂CH₂CH₂OCH₂CH₂OH | —NHCH₂CH₂CH₂OMe |
| —OCH₂CH₂CH₂OCH₂CH₂OMe | —NHCH₂CH₂CH₂OEt |
| —OCH₂CH₂CH₂OCH₂CH₂OEt | —NHCH₂CH₂CH₂NH₂ |
| —OCH₂CH₂CH₂OCH₂CH₂NH₂ | —NHCH₂CH₂CH₂NHMe |
| —OCH₂CH₂CH₂OCH₂CH₂NHMe | —NHCH₂CH₂CH₂NMe₂ |
| —OCH₂CH₂CH₂OCH₂CH₂NMe₂ | 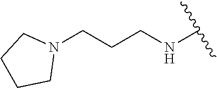 [SMILES: *NCCCN1CCCC1] |
| 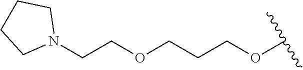 [SMILES: *OCCCOCCN1CCCC1] | 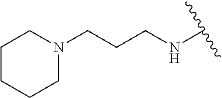 [SMILES: *NCCCN1CCCCC1] |
| 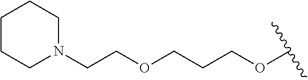 [SMILES: *OCCCOCCN1CCCCC1] | 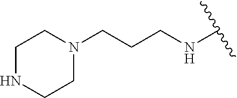 [SMILES: *NCCCN1CCNCC1] |
| 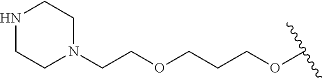 [SMILES: *OCCCOCCN1CCNCC1] | 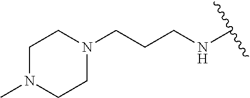 [SMILES: *NCCCN1CCN(C)CC1] |
| 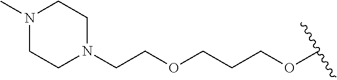 [SMILES: *OCCCOCCN1CCN(C)CC1] | 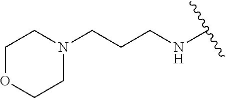 [SMILES: *NCCCN1CCOCC1] |
| 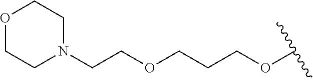 [SMILES: *OCCCOCCN1CCOCC1] | —NHCH₂CH₂OCH₂CH₂OH |
| —OCH₂CH₂CH₂OCH₂CH₂CH₂OH | —NHCH₂CH₂OCH₂CH₂OMe |
| —OCH₂CH₂CH₂OCH₂CH₂CH₂OMe | —NHCH₂CH₂OCH₂CH₂OEt |
| —OCH₂CH₂CH₂OCH₂CH₂CH₂OEt | —NHCH₂CH₂OCH₂CH₂NH₂ |
| —OCH₂CH₂CH₂OCH₂CH₂CH₂NH₂ | —NHCH₂CH₂OCH₂CH₂NHMe |
| —OCH₂CH₂CH₂OCH₂CH₂CH₂NHMe | —NHCH₂CH₂OCH₂CH₂NMe₂ |
| —OCH₂CH₂CH₂OCH₂CH₂CH₂NMe₂ | —N(CH₂CH₂OH)₂ |
| 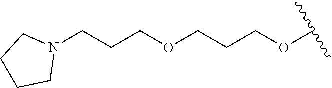 [SMILES: *OCCCOCCCN1CCCC1] | —N(CH₂CH₂OMe)₂ |
| 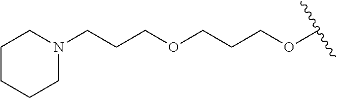 [SMILES: *OCCCOCCCN1CCCCC1] | —N(CH₂CH₂OEt)₂ |
| 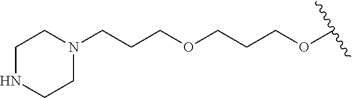 [SMILES: *OCCCOCCCN1CCNCC1] | —N(CH₂CH₂CH₂OH)₂ |
| 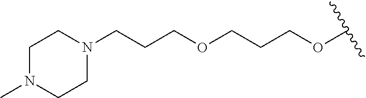 [SMILES: *OCCCOCCCN1CCN(C)CC1] | —N(CH₂CH₂CH₂OMe)₂ |
| 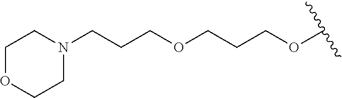 [SMILES: *OCCCOCCCN1CCOCC1] | —N(CH₂CH₂CH₂OEt)₂ |
| —OCH₂CH₂NHCH₂CH₂OH | —N(CH₂CH₂CH₂NH₂)₂ |
| —OCH₂CH₂NHCH₂CH₂OMe | —N(CH₂CH₂CH₂NHMe)₂ |
| —OCH₂CH₂NHCH₂CH₂OEt | —N(CH₂CH₂CH₂NMe₂)₂ |
| —OCH₂CH₂NHCH₂CH₂NH₂ | 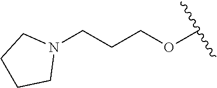 [SMILES: *OCCCN1CCCC1] |
| —OCH₂CH₂NHCH₂CH₂NHMe | 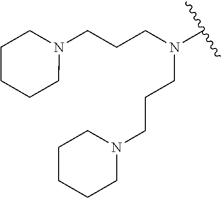 [SMILES: *N(CCCN1CCCCC1)CCCN1CCCCC1] |
| —OCH₂CH₂NHCH₂CH₂NMe₂ | 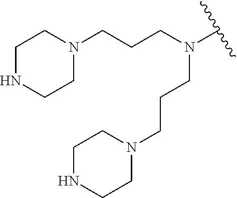 [SMILES: *N(CCCN1CCNCC1)CCCN1CCNCC1] |
| 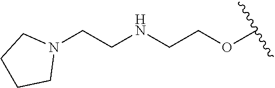 [SMILES: *OCCNCCN1CCCC1] | 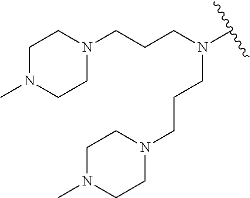 [SMILES: *N(CCCN1CCN(C)CC1)CCCN1CCN(C)CC1] |
| 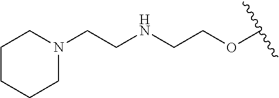 [SMILES: *OCCNCCN1CCCCC1] | 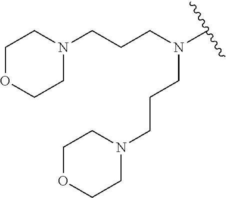 [SMILES: *N(CCCN1CCOCC1)CCCN1CCOCC1] |
| 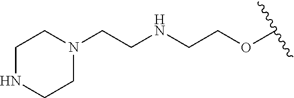 [SMILES: *OCCNCCN1CCNCC1] | —N(CH₂CH₂OCH₂CH₂OH)₂ |
| 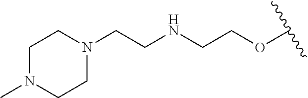 [SMILES: *OCCNCCN1CCN(C)CC1] | —N(CH₂CH₂OCH₂CH₂OMe)₂ |
| 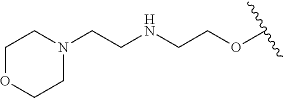 [SMILES: *OCCNCCN1CCOCC1] | —N(CH₂CH₂OCH₂CH₂OEt)₂ |
| —OCH₂CH₂NHCH₂CH₂CH₂OH | —N(CH₂CH₂OCH₂CH₂NH₂)₂ |
| —OCH₂CH₂NHCH₂CH₂CH₂OMe | —N(CH₂CH₂OCH₂CH₂NHMe)₂ |
| —OCH₂CH₂NHCH₂CH₂CH₂OEt | —N(CH₂CH₂OCH₂CH₂NMe₂)₂ |
| —OCH₂CH₂NHCH₂CH₂CH₂NH₂ | —NMeCH₂CH₂OH |
| —OCH₂CH₂NHCH₂CH₂CH₂NHMe | —NMeCH₂CH₂OMe |
| —OCH₂CH₂NHCH₂CH₂CH₂NMe₂ | —NMeCH₂CH₂OEt |
| 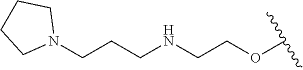 [SMILES: *OCCNCCCN1CCCC1] | —NMeCH₂CH₂NH₂ |
| 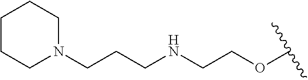 [SMILES: *OCCNCCCN1CCCCC1] | —NMeCH₂CH₂NHMe |
| 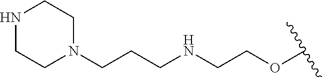 [SMILES: *OCCNCCCN1CCNCC1] | —NMeCH₂CH₂NMe₂ |
| 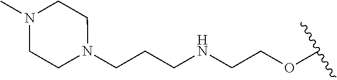 [SMILES: *OCCNCCCN1CCN(C)CC1] | 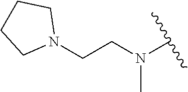 [SMILES: *N(C)CCN1CCCC1] |
| 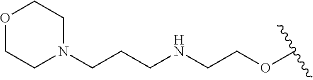 [SMILES: *OCCNCCCN1CCOCC1] | 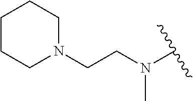 [SMILES: *N(C)CCN1CCCCC1] |
| —OCH₂CH₂CH₂NHCH₂CH₂OH | 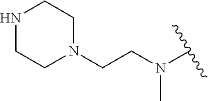 [SMILES: *N(C)CCN1CCNCC1] |
| —OCH₂CH₂CH₂NHCH₂CH₂OMe | 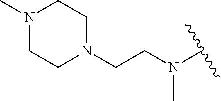 [SMILES: *N(C)CCN1CCN(C)CC1] |
| —OCH₂CH₂CH₂NHCH₂CH₂OEt | 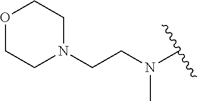 [SMILES: *N(C)CCN1CCOCC1] |
| —OCH₂CH₂CH₂NHCH₂CH₂NH₂ | —NMeCH₂CH₂CH₂OH |
| —OCH₂CH₂CH₂NHCH₂CH₂NHMe | —NMeCH₂CH₂CH₂OMe |
| —OCH₂CH₂CH₂NHCH₂CH₂NMe₂ | —NMeCH₂CH₂CH₂OEt |
| 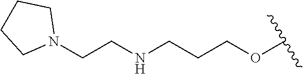 [SMILES: *OCCCNCCN1CCCC1] | —NMeCH₂CH₂CH₂NH₂ |
| 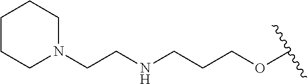 [SMILES: *OCCCNCCN1CCCCC1] | —NMeCH₂CH₂CH₂NHMe |
| 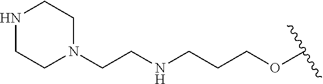 [SMILES: *OCCCNCCN1CCNCC1] | —NMeCH₂CH₂CH₂NMe₂ |
| 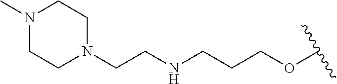 [SMILES: *OCCCNCCN1CCN(C)CC1] | 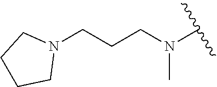 [SMILES: *N(C)CCCN1CCCC1] |
| 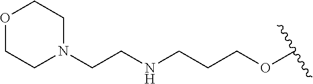 [SMILES: *OCCCNCCN1CCOCC1] | 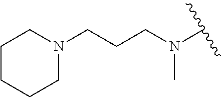 [SMILES: *N(C)CCCN1CCCCC1] |
| —OCH₂CH₂CH₂NHCH₂CH₂CH₂OH | 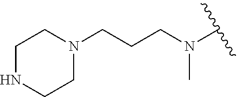 [SMILES: *N(C)CCCN1CCNCC1] |
| —OCH₂CH₂CH₂NHCH₂CH₂CH₂OMe | 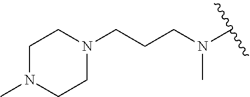 [SMILES: *N(C)CCCN1CCN(C)CC1] |
| —OCH₂CH₂CH₂NHCH₂CH₂CH₂OEt | 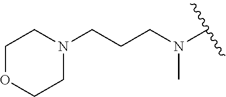 [SMILES: *N(C)CCCN1CCOCC1] |
| —OCH₂CH₂CH₂NHCH₂CH₂CH₂NH₂ | —NMeCH₂CH₂OCH₂CH₂OH |
| —OCH₂CH₂CH₂NHCH₂CH₂CH₂NHMe | —NMeCH₂CH₂OCH₂CH₂OMe |
| —OCH₂CH₂CH₂NHCH₂CH₂CH₂NMe₂ | —NMeCH₂CH₂OCH₂CH₂OEt |
| 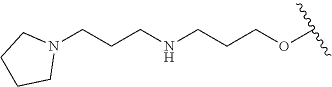 [SMILES: *OCCCNCCCN1CCCC1] | —NMeCH₂CH₂OCH₂CH₂NH₂ |
| 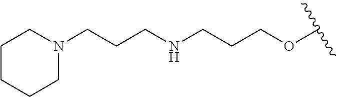 [SMILES: *OCCCNCCCN1CCCCC1] | —NMeCH₂CH₂OCH₂CH₂NHMe |
| 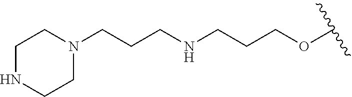 [SMILES: *OCCCNCCCN1CCNCC1] | —NMeCH₂CH₂OCH₂CH₂NMe₂ |
| 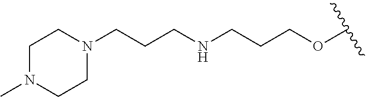 [SMILES: *OCCCNCCCN1CCN(C)CC1] | —O(C═O)OMe |
| 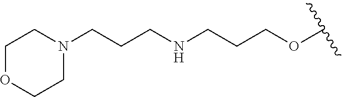 [SMILES: *OCCCNCCCN1CCOCC1] | —O(C═O)OEt |
| —OCH₂CH₂N(CH₂CH₂OH)₂ | —O(C═O)OnPr |
| —OCH₂CH₂N(CH₂CH₂OMe)₂ | —O(C═O)OiPr |
| —OCH₂CH₂N(CH₂CH₂OEt)₂ | —O(C═O)OcPr |
| —OCH₂CH₂N(CH₂CH₂CH₂OH)₂ | —O(C═O)OcBu |
| —OCH₂CH₂N(CH₂CH₂CH₂OMe)₂ | —O(C═O)OcPn |
| —OCH₂CH₂N(CH₂CH₂CH₂OEt)₂ | —O(C═O)OcHex |
| —OCH₂CH₂CH₂(HCH₂CH₂OH)₂ | —COOH |
| —OCH₂CH₂CH₂N(CH₂CH₂OMe)₂ | —CH₂(C═O)OH |
| —OCH₂CH₂CH₂N(CH₂CH₂OEt)₂ | —CH₂(C═O)OMe |
| —OCH₂CH₂CH₂N(CH₂CH₂CH₂OH)₂ | —CH₂(C═O)OEt |
| —OCH₂CH₂CH₂N(CH₂CH₂CH₂OMe)₂ | —CH₂(C═O)OnPr |
| —OCH₂CH₂CH₂N(CH₂CH₂CH₂OEt)₂ | —CH₂(C═O)OiPr |
| —OCH₂CH₂NMeCH₂CH₂OH | —CH₂(C═O)OcPr |
| —OCH₂CH₂NMeCH₂CH₂OMe | —CH₂(C═O)OcBu |
| —OCH₂CH₂NMeCH₂CH₂OEt | —CH₂(C═O)OcPn |
| —OCH₂CH₂NMeCH₂CH₂NH₂ | —CH₂(C═O)OcHex |
| —OCH₂CH₂NMeCH₂CH₂NHMe | —OCH₂(C═O)OH |
| —OCH₂CH₂NMeCH₂CH₂NMe₂ | —OCH₂(C═O)OMe |
| 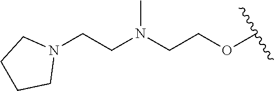 [SMILES: *OCCN(C)CCN1CCCC1] | —OCH₂(C═O)OEt |
| 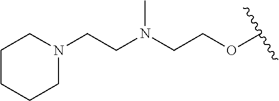 [SMILES: *OCCN(C)CCN1CCCCC1] | —OCH₂(C═O)OnPr |
| 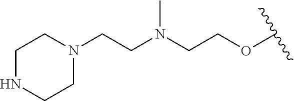 [SMILES: *OCCN(C)CCN1CCNCC1] | —OCH₂(C═O)OiPr |
| 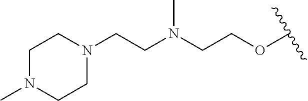 [SMILES: *OCCN(C)CCN1CCN(C)CC1] | —OCH₂(C═O)OcPr |
| 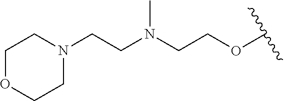 [SMILES: *OCCN(C)CCN1CCOCC1] | —OCH₂(C═O)OcBu |
| —OCH₂CH₂NMeCH₂CH₂CH₂OH | —OCH₂(C═O)OcPn |
| —OCH₂CH₂NMeCH₂CH₂CH₂OMe | —OCH₂(C═O)OcHex |
| —OCH₂CH₂NMeCH₂CH₂CH₂OEt | —OCH₂(C═O)OH |
| —OCH₂CH₂NMeCH₂CH₂CH₂NH₂ | —OCH₂(C═O)OMe |
| —OCH₂CH₂NMeCH₂CH₂CH₂NHMe | —OCH₂(C═O)OEt |
| —OCH₂CH₂NMeCH₂CH₂CH₂NMe₂ | —OCH₂(C═O)OnPr |
| 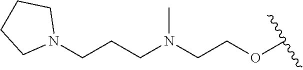 [SMILES: *OCCN(C)CCCN1CCCC1] | —OCH₂(C═O)OiPr |
| 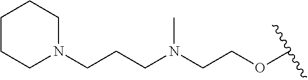 [SMILES: *OCCN(C)CCCN1CCCCC1] | —OCH₂(C═O)OcPr |
| 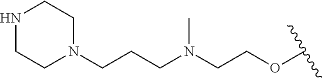 [SMILES: *OCCN(C)CCCN1CCNCC1] | —OCH₂(C═O)OcBu |
| 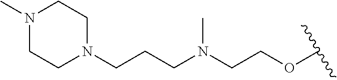 [SMILES: *OCCN(C)CCCN1CCN(C)CC1] | —OCH₂(C═O)OcPn |
| 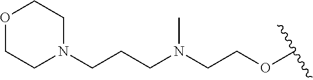 [SMILES: *OCCN(C)CCCN1CCOCC1] | —OCH₂(C═O)OcHex |
| —OCH₂CH₂CH₂NMeCH₂CH₂OH | —NHCH₂(C═O)OH |
| —OCH₂CH₂CH₂NMeCH₂CH₂OMe | —NHCH₂(C═O)OMe |
| —OCH₂CH₂CH₂NMeCH₂CH₂OEt | —NHCH₂(C═O)OEt |
| —OCH₂CH₂CH₂NMeCH₂CH₂NH₂ | —NHCH₂(C═O)OnPr |
| —OCH₂CH₂CH₂NMeCH₂CH₂NHMe | —NHCH₂(C═O)OiPr |
| —OCH₂CH₂CH₂NMeCH₂CH₂NMe₂ | —NHCH₂(C═O)OcPr |
| 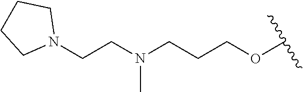 [SMILES: *OCCCN(C)CCN1CCCC1] | —NHCH₂(C═O)OcBu |
| 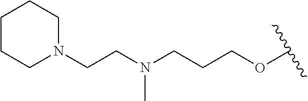 [SMILES: *OCCCN(C)CCN1CCCCC1] | —NHCH₂(C═O)OcPn |
| 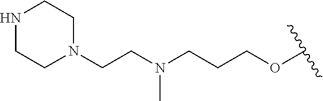 [SMILES: *OCCCN(C)CCN1CCNCC1] | —NHCH₂(C═O)OcHex |
| 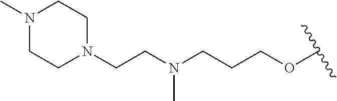 [SMILES: *OCCCN(C)CCN1CCN(C)CC1] | —NMeCH₂(C═O)OH |
| 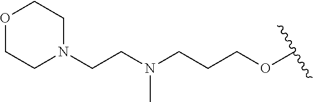 [SMILES: *OCCCN(C)CCN1CCOCC1] | —NH(CH₂(C═O)OH)₂ |
| —OCH₂CH₂CH₂NMeCH₂CH₂CH₂OH | —CH₂CH₂(C═O)OH |
| —OCH₂CH₂CH₂NMeCH₂CH₂CH₂OMe | —CH₂OCH₂(C═O)OH |
| —OCH₂CH₂CH₂NMeCH₂CH₂CH₂OEt | —OCH₂CH₂(C═O)OH |
| —OCH₂CH₂CH₂NMeCH₂CH₂CH₂NH₂ | —CH₂CH₂CH₂(C═O)OH |
| —OCH₂CH₂CH₂NMeCH₂CH₂CH₂NHMe | —CH₂CH₂OCH₂(C═O)OH |
| —OCH₂CH₂CH₂NMeCH₂CH₂CH₂NMe₂ | —CH₂OCH₂CH₂(C═O)OH |
| 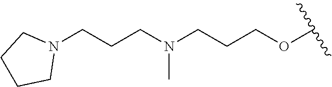 [SMILES: *OCCCN(C)CCCN1CCCC1] | —OCH₂CH₂CH₂(C═O)OH |
| 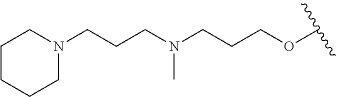 [SMILES: *OCCCN(C)CCCN1CCCCC1] | —OCH₂CH₂OCH₂(C═O)OH |
| 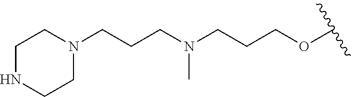 [SMILES: *OCCCN(C)CCCN1CCNCC1] | —CH₂CH₂CH₂CH₂(C═O)OH |
| 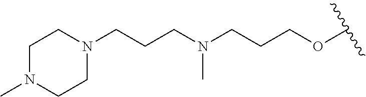 [SMILES: *OCCCN(C)CCCN1CCN(C)CC1] | —CH₂CH₂CH₂OCH₂(C═O)OH |
| 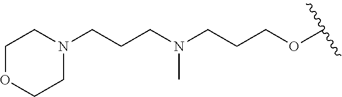 [SMILES: *OCCCN(C)CCCN1CCOCC1] | —CH₂CH₂OCH₂CH₂(C═O)OH |
| —CH₂OH | —CH₂OCH₂CH₂CH₂(C═O)OH |
| —CH₂OMe | —OCH₂CH₂CH₂CH₂(C═O)OH |
| —CH₂OEt | —CH₂OCH₂CH₂OCH₂(C═O)OH |
| —CH₂NH₂ | —OCH₂CH₂CH₂OCH₂(C═O)OH |
| —CH₂NHMe | —OCH₂CH₂OCH₂CH₂(C═O)OH |
| —CH₂NMe₂ | —CH₂OCH₂CH₂CH₂(C═O)OH |
| 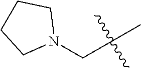 [SMILES: *CN1CCCC1] | —CH₂CH₂CH₂CH₂CH₂(C═O)OH |
| 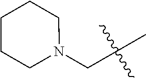 [SMILES: *CN1CCCCC1] | —CH₂CH₂CH₂CH₂OCH₂(C═O)OH |
| 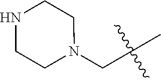 [SMILES: *CN1CCNCC1] | —CH₂CH₂CH₂OCH₂CH₂(C═O)OH |
| 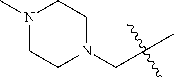 [SMILES: *CN1CCN(C)CC1] | —CH₂CH₂OCH₂CH₂CH₂(C═O)OH |
| 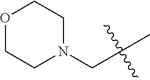 [SMILES: *CN1CCOCC1] | —CH₂OCH₂CH₂CH₂CH₂(C═O)OH |
| —CH₂CH₂OH | —OCH₂CH₂CH₂CH₂CH₂(C═O)OH |
| —CH₂CH₂OMe | —CH₂CH₂OCH₂CH₂OCH₂(C═O)OH |
| —CH₂CH₂OEt | —CH₂OCH₂CH₂CH₂OCH₂(C═O)OH |
| —CH₂CH₂NH₂ | —OCH₂CH₂CH₂CH₂OCH₂(C═O)OH |
| —CH₂CH₂NHMe | —CH₂OCH₂CH₂OCH₂CH₂(C═O)OH |
| —CH₂CH₂NMe₂ | —OCH₂CH₂CH₂OCH₂CH₂(C═O)OH |
| 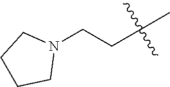 [SMILES: *CCN1CCCC1] | —OCH₂CH₂OCH₂CH₂CH₂(C═O)OH |
| 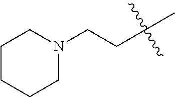 [SMILES: *CCN1CCCCC1] | —OCH₂CH₂OCH₂CH₂OCH₂(C═O)OH |
| 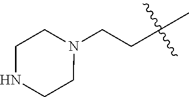 [SMILES: *CCN1CCNCC1] | —CH₂CH₂CH₂CH₂CH₂CH₂(C═O)OH |
| 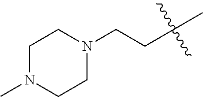 [SMILES: *CCN1CCN(C)CC1] | —CH₂CH₂CH₂CH₂CH₂OCH₂(C═O)OH |
| 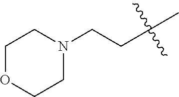 [SMILES: *CCN1CCOCC1] | —CH₂CH₂CH₂OCH₂CH₂OCH₂(C═O)OH |
| —CH₂CH₂CH₂OH | —CH₂OCH₂CH₂OCH₂CH₂OCH₂C(O)OH |
| —CH₂CH₂CH₂OMe | —OCH₂CH₂CH₂OCH₂CH₂OCH₂C(O)OH |
| —CH₂CH₂CH₂OEt | —CH₂CH₂CH₂CH₂OCH₂CH₂(C═O)OH |
| —CH₂CH₂CH₂NH₂ | —CH₂CH₂OCH₂CH₂OCH₂CH₂(C═O)OH |
| —CH₂CH₂CH₂NHMe | —CH₂OCH₂CH₂CH₂OCH₂CH₂(C═O)OH |
| —CH₂CH₂CH₂NMe₂ | —OCH₂CH₂CH₂CH₂OCH₂CH₂(C═O)OH |
| 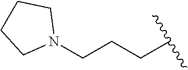 [SMILES: *CCCN1CCCC1] | —OCH₂CH₂OCH₂CH₂OCH₂CH₂(CO)OH |
| 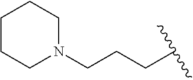 [SMILES: *CCCN1CCCCC1] | —CH₂CH₂CH₂OCH₂CH₂CH₂(C═O)OH |
| 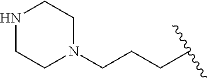 [SMILES: *CCCN1CCNCC1] | —CH₂OCH₂CH₂OCH₂CH₂CH₂(C═O)OH |
| 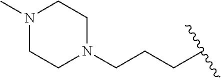 [SMILES: *CCCN1CCN(C)CC1] | —OCH₂CH₂CH₂OCH₂CH₂CH₂(C═O)OH |
| 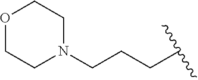 [SMILES: *CCCN1CCOCC1] | —CH₂CH₂OCH₂CH₂CH₂CH₂(C═O)OH |
| —CH₂CH₂CH₂CH₂OH | —OCH₂CH₂OCH₂CH₂CH₂CH₂(C═O)OH |
| —CH₂CH₂CH₂CH₂OMe | —CH₂OCH₂CH₂CH₂CH₂CH₂(C═O)OH |
| —CH₂CH₂CH₂CH₂OEt | —OCH₂CH₂CH₂CH₂CH₂CH₂(C═O)OH |
| —CH₂CH₂CH₂CH₂NH₂ | —NHCH₂CH₂(C═O)OH |
| —CH₂CH₂CH₂CH₂NHMe | —NMeCH₂CH₂(C═O)OH |
| —CH₂CH₂CH₂CH₂NMe₂ | —N(CH₂CH₂(C═O)OH)₂ |
| 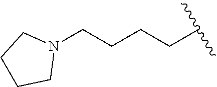 [SMILES: *CCCCN1CCCC1] | —NHCH₂CH₂OCH₂(C═O)OH |
| 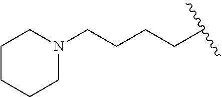 [SMILES: *CCCCN1CCCCC1] | —NMeCH₂CH₂OCH₂(C═O)OH |
| 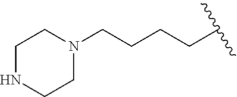 [SMILES: *CCCCN1CCNCC1] | —N(CH₂CH₂OCH₂(C═O)OH)₂ |
| 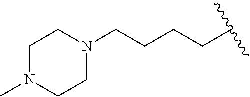 [SMILES: *CCCCN1CCN(C)CC1] | —NHCH₂CH₂CH₂CH₂(C═O)OH |
| 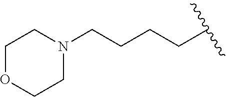 [SMILES: *CCCCN1CCOCC1] | —NMeCH₂CH₂CH₂CH₂(C═O)OH |
| —CH₂OCH₂CH₂OH | —N(CH₂CH₂CH₂CH₂(C═O)OH)₂ |
| —CH₂OCH₂CH₂OMe | —NHCH₂CH₂CH₂OCH₂(C═O)OH |
| —CH₂OCH₂CH₂OEt | —NMeCH₂CH₂CH₂OCH₂(C═O)OH |
| —CH₂OCH₂CH₂NH₂ | —N(CH₂CH₂CH₂OCH₂(C═O)OH)₂ |
| —CH₂OCH₂CH₂NHMe | —NHCH₂CH₂OCH₂CH₂(C═O)OH |
| —CH₂OCH₂CH₂NMe₂ | —NMeCH₂CH₂OCH₂CH₂(C═O)OH |
| 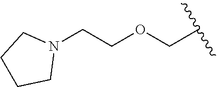 [SMILES: *COCCN1CCCC1] | —N(CH₂CH₂OCH₂CH₂(C═O)OH)₂ |
| 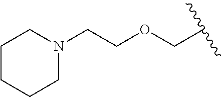 [SMILES: *COCCN1CCCCC1] | —NHCH₂CH₂CH₂CH₂CH₂(C═O)OH |
| 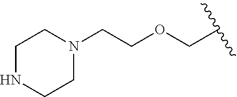 [SMILES: *COCCN1CCNCC1] | —NMeCH₂CH₂CH₂CH₂CH₂(C═O)OH |
| 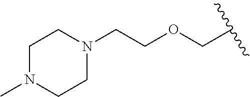 [SMILES: *COCCN1CCN(C)CC1] | —N(CH₂CH₂CH₂CH₂CH₂(C═O)OH)₂ |
| 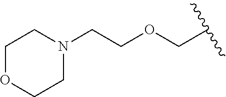 [SMILES: *COCCN1CCOCC1] | —NHCH₂CH₂CH₂CH₂OCH₂(C═O)OH |
| —CH₂NHCH₂CH₂OH | —NMeCH₂CH₂CH₂CH₂OCH₂(C═O)OH |
| —CH₂NHCH₂CH₂OMe | —N(CH₂CH₂CH₂CH₂OCH₂(C═O)OH)₂ |
| —CH₂NHCH₂CH₂OEt | —NHCH₂CH₂CH₂OCH₂CH₂(C═O)OH |
| —CH₂NHCH₂CH₂NH₂ | —NMeCH₂CH₂CH₂OCH₂CH₂(C═O)OH |
| —CH₂NHCH₂CH₂NHMe | —N(CH₂CH₂CH₂OCH₂CH₂(C═O)OH)₂ |
| —CH₂NHCH₂CH₂NMe₂ | —NHCH₂CH₂OCH₂CH₂CH₂(C═O)OH |
| 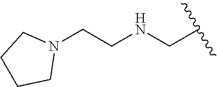 [SMILES: *CNCCN1CCCC1] | —NMeCH₂CH₂OCH₂CH₂CH₂(C═O)OH |
| 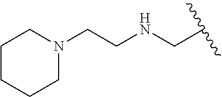 [SMILES: *CNCCN1CCCCC1] | —N(CH₂CH₂OCH₂CH₂CH₂(C═O)OH)₂ |
| 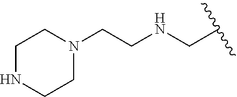 [SMILES: *CNCCN1CCNCC1] | —NHCH₂CH₂OCH₂CH₂OCH₂(C═O)OH |
| 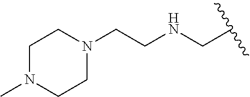 [SMILES: *CNCCN1CCN(C)CC1] | —NMeCH₂CH₂OCH₂CH₂OCH₂(C═O)OH |
| 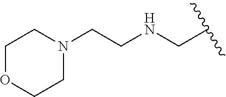 [SMILES: *CNCCN1CCOCC1] | —N(CH₂CH₂OCH₂CH₂OCH₂(C═O)OH)₂ |
| —CH₂N(CH₂CH₂OH)₂ | —NHCH₂CH₂CH₂OCH₂CH₂OCH₂CO₂H |
| —CH₂N(CH₂CH₂OMe)₂ | —NMeCH₂CH₂CH₂OCH₂CH₂OCH₂CO₂H |
| —CH₂N(CH₂CH₂OEt)₂ | —N(CH₂CH₂CH₂OCH₂CH₂OCH₂CO₂H)₂ |
| —CH₂NMeCH₂CH₂OH | —NHCH₂CH₂CH₂CH₂OCH₂CH₂CO₂H |
| —CH₂NMeCH₂CH₂OMe | —NMeCH₂CH₂CH₂CH₂OCH₂CH₂CO₂H |
| —CH₂NMeCH₂CH₂OEt | —N(CH₂CH₂CH₂CH₂OCH₂CH₂CO₂H)₂ |
| —CH₂NMeCH₂CH₂NH₂ | —NHCH₂CH₂OCH₂CH₂OCH₂CH₂CO₂H |
| —CH₂NMeCH₂CH₂NHMe | —NMeCH₂CH₂OCH₂CH₂OCH₂CH₂CO₂H |
| —CH₂NMeCH₂CH₂NMe₂ | —N(CH₂CH₂OCH₂CH₂OCH₂CH₂CO₂H)₂ |
| 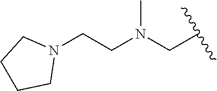 [SMILES: *CN(C)CCN1CCCC1] | —NHCH₂CH₂CH₂OCH₂CH₂CH₂CO₂H |
| 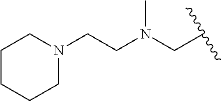 [SMILES: *CN(C)CCN1CCCCC1] | —NMeCH₂CH₂CH₂OCH₂CH₂CH₂CO₂H |
| 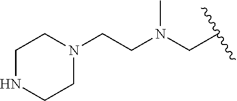 [SMILES: *CN(C)CCN1CCNCC1] | —N(CH₂CH₂CH₂OCH₂CH₂CH₂CO₂H)₂ |
| 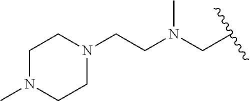 [SMILES: *CN(C)CCN1CCN(C)CC1] | —NHCH₂CH₂OCH₂CH₂CH₂CH₂CO₂H |
| 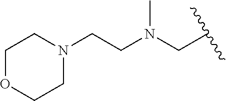 [SMILES: *CN(C)CCN1CCOCC1] | —NMeCH₂CH₂OCH₂CH₂CH₂CH₂CO₂H |
| —CH₂CH₂OCH₂CH₂OH | —N(CH₂CH₂OCH₂CH₂CH₂CH₂CO₂H)₂ |
| —CH₂CH₂OCH₂CH₂OMe | —NHCH₂CH₂CH₂CH₂CH₂CH₂CO₂H |
| —CH₂CH₂OCH₂CH₂OEt | —NMeCH₂CH₂CH₂CH₂CH₂CH₂CO₂H |
| —CH₂CH₂OCH₂CH₂NH₂ | —N(CH₂CH₂CH₂CH₂CH₂CH₂CO₂H)₂. |
| —CH₂CH₂OCH₂CH₂NHMe | |

14. The compound of claim 1, having the structure: 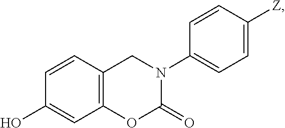
wherein Z is selected from the group consisting of

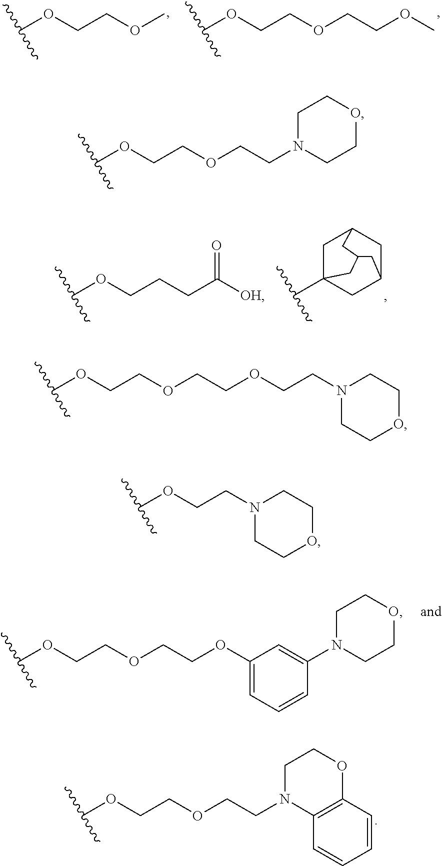

15. The compound of claim 1, having the structure: 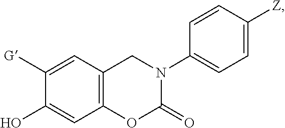
wherein Z is selected from the group consisting of 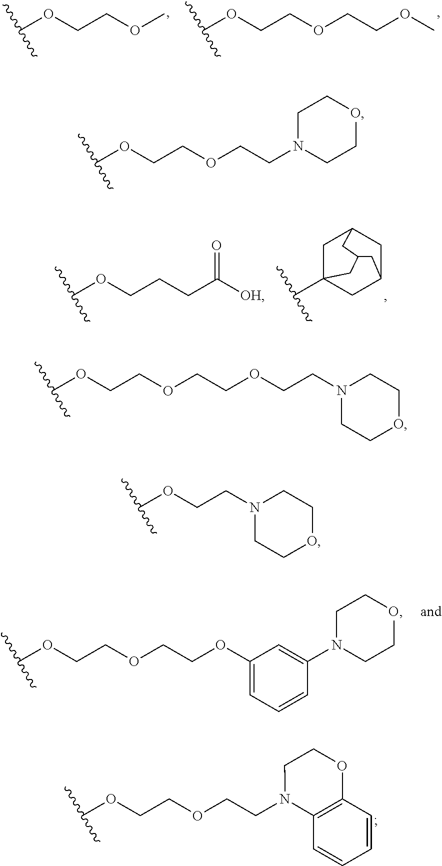
and
wherein G′ is halogen.

16. The compound of claim 15, wherein G′ is F.

17. The compound of claim 1, which is selected from the group consisting of:

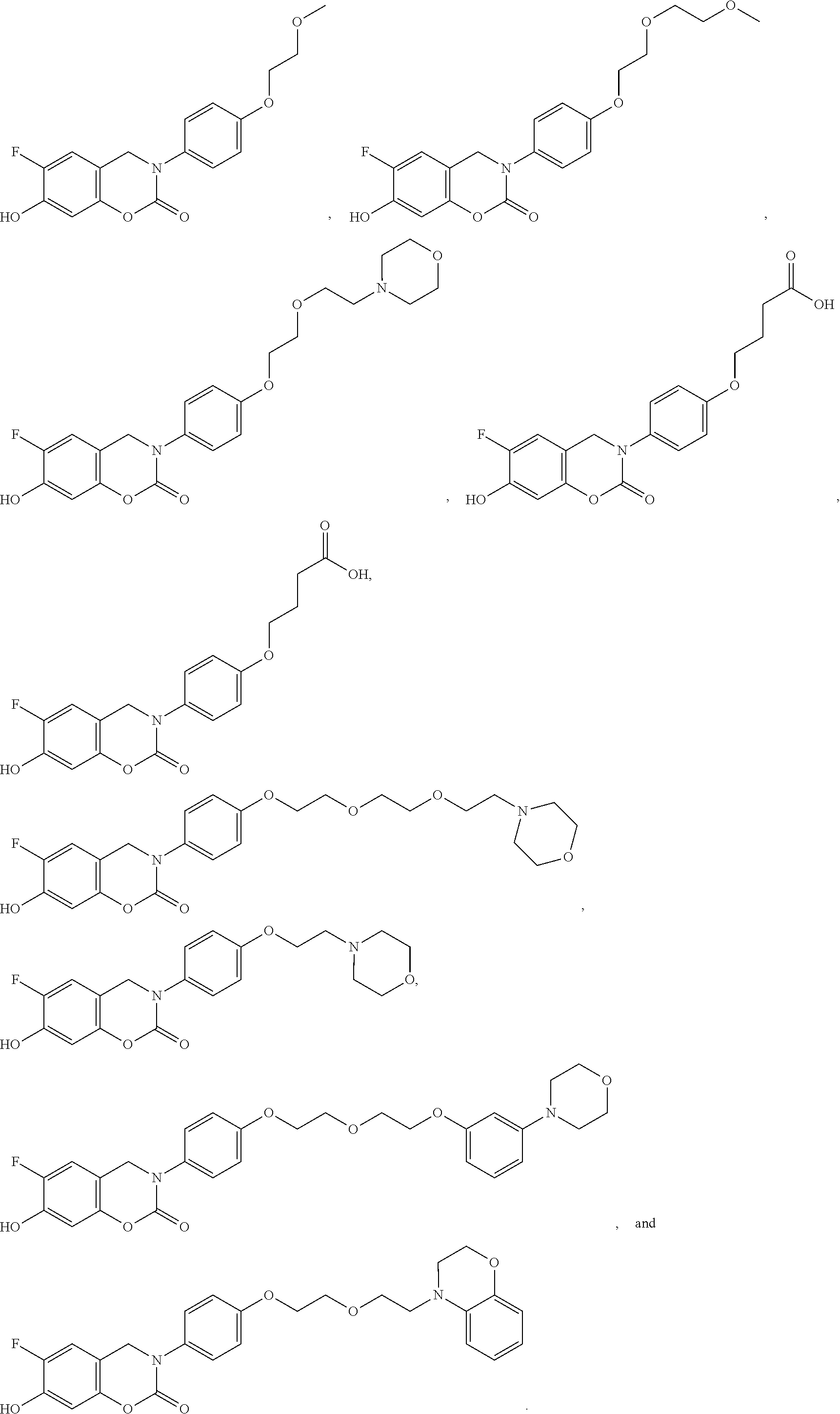

18. A pharmaceutical composition comprising the compound of claim 1, or a salt, enantiomer, tautomer, or solvate thereof, and a pharmaceutically acceptable carrier.

19. A method of inhibiting macrophage migration inhibitory factor (MIF) activity in a subject, the method comprising:
administering to the subject an effective amount of the compound of claim 1, or a salt, enantiomer, tautomer, or solvate thereof.

20. A method of treating a disease or condition in which inhibition of macrophage migration inhibitory factor (MIF) activity in a subject is therapeutically beneficial, the method comprising administering to the subject an effective amount of a composition comprising the compound of claim 1, or a salt, enantiomer, tautomer, or solvate thereof,
wherein the disease or condition is selected from the group consisting of an inflammatory disease, an autoimmune disease, and cancer.

21. The method of claim 20, wherein at least one of the following applies:
(a) the inflammatory disease or condition is selected from the group consisting of proliferative vascular disease, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Berger's disease, Retier's syndrome, and Hodgkins disease;
(b) the autoimmune disease is selected from the group consisting of multiple sclerosis, systemic lupus erythematosus, rheumatoid arthritis, graft versus host disease, autoimmune pulmonary inflammation, autoimmune encephalomyelitis, Guillain-Barre syndrome, autoimmune thyroiditis, insulin dependent diabetes mellitus, Crohn's disease, scleroderma, psoriasis, Sjögren's syndrome, and autoimmune inflammatory eye disease;
(c) the cancer is a solid tumor or a hematological cancer;
(d) the cancer is selected from the group consisting of prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head or neck, thyroid cancer, glioblastoma, sarcoma, bladder cancer, lymphoma, leukemia, diffuse large B-cell lymphoma (DLBCL), mantle cell lymphoma, non-Hodgkin lymphoma, Hodgkin lymphoma, and multiple myeloma.

22-24. (canceled)

25. The method of claim 20, wherein the composition is formulated for an administration route selected from the group consisting of oral, transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

26. The method of claim 25, wherein the composition is formulated for oral administration.

27. (canceled)
